(19) United States
(12) Patent Application Publication
(10) Pub. No.: US 2018/0049999 A1
(43) Pub. Date: Feb. 22, 2018

(54) COMPOSITIONS AND METHODS OF TREATMENT OF BREAST DISORDERS AND ESTROGEN-RELATED DISORDERS

(71) Applicant: Atossa Genetics Inc., Seattle, WA (US)

(72) Inventor: Steven C. Quay, Seattle, WA (US)

(21) Appl. No.: 15/561,249

(22) PCT Filed: Apr. 6, 2016
(86) PCT No.: PCT/US16/26170

§ 371 Date: Sep. 25, 2017

Related U.S. Application Data
(60) Provisional application No. 62/147,536, filed on Apr. 14, 2015.

(51) Int. Cl. A61K 31/138; A61K 31/565; A61K 31/4196; A61K 31/202; A61K 31/593; A61K 31/592

(52) U.S. Cl. CPC A61K 31/138; A61K 31/565; A61K 31/4196; A61K 2300/00; A61K 31/593; A61K 31/592; A61K 31/202

(57) ABSTRACT

The present invention relates to pharmaceutical compositions comprising at least one therapeutic agent, a fatty acid mixture comprising at least one omega-3 fatty acid, and at least one vitamin D compound. Also described are methods of preparation of such compositions, and methods of prevention and treatment of breast disorders and estrogen-related disorders.

### CROSS-REFERENCE

This application claims the benefit of U.S. Provisional Application No. 62/147,536, filed Apr. 14, 2015, which application is incorporated herein by reference in its entirety for all purposes.

### FIELD OF THE INVENTION

The application generally relates to pharmaceutical compositions that are useful for the treatment of subjects in risk for or having breast disorders and estrogen-related disorder and methods of using such compositions.

### BACKGROUND OF THE INVENTION

There are 2.5 million women alive in U.S., who have a history of breast cancer, of which approximately 70% of human breast cancers are hormone-dependent and estrogen receptor-positive (ER+) and 88% are post-menopausal. Typically, treatment is interventional (surgery, chemotherapy, radiation) followed by adjuvant therapy when appropriate. A significant improvement in post-surgical survival and a reduction in disease relapse has been observed, especially in women with early stage breast cancers and those with ER+ tumors, who receive adjuvant systemic therapy with Selective Estrogen Receptor Modulators (SERMs), Selective Estrogen Receptor Downregulators (SERDs) or Aromatase Inhibitors (AIs) alone or in combination with cytotoxic therapy.

Tamoxifen, the first SERM to be approved by FDA for the treatment and prevention of breast cancers, is available as an oral formulation either as a once a day pill (Novaldex™) or in liquid formulation (Soltamax®). While tamoxifen acts as an antiestrogen in breast tissue, it also functions as an estrogen-agonist in a variety of other tissues, for example, in bone and in ovary. Due to the systemic presence of tamoxifen, women undergoing treatment with tamoxifen experience disease relapse and severe side effects that include endometrial and ovarian cancer, blood clots, deep vein thrombosis, bone loss and osteoporosis, and stroke. Concerns regarding tamoxifen-related toxicity limits its use as a preventive agent and has been the driving force for the search for other SERMs with less toxicity and the development of 4-Hydroxytamoxifen (4-OHT), raloxifene (Trade name: Evista), toremifene (Trade name: Fareston®), bazedofoxifene (Conbriza®), and lasofoxifene (Fablyn®). When used as systemic therapy, these newer SERMs also have severe side effects that include increased risk of venous thromboembolic events such as deep vein thrombosis and pulmonary embolism, and increased risk of death due to coronary artery heart disease.

Fulvestrant (Trade name: Faslodex), a non-steroidal antiestrogen that binds and targets the estrogen receptor for destruction is the best studied amongst the SERDs, and is approved by FDA for the treatment and prevention of breast cancer as a once a day pill in an oral formulation. Unlike tamoxifen, fulvestrant rarely has estrogen-like like effects on bone and ovary. However, the women undergoing treatment with fulvestrant experience severe gastrointestinal symptoms and bone pain.

Currently, several AI drugs (anastrozole (Aridimix®), exemestrane (Arosmasin®), and letrozole (Femara®)), are the treatment of choice for postmenopausal women. Although results from meta-analyses of AI monotherapy and sequenced therapy (switch from tamoxifen to AI) clinical trials show that disease-free survival was significantly improved, and that overall survival was prolonged for patients who switched from tamoxifen to AI therapy, risk of fractures and muscle and bone pain is significantly increased with AI.

The first line therapy with systemic SERMs, SERDs, and AI thus results in low compliance, low adherence, and low persistence (Nonadherence to Adjuvant Tamoxifen Therapy in Women with Primary Breast Cancer, Partridge et al., J. Clinical Oncology, Vol. 21, Feb. 15, 2003; Adherence to Initial adjuvant anastrozole therapy among women with early stage breast cancer, Partridge et al., J. Clinical Oncology, Vol. 26, No. 4, Feb. 1, 2008; Early Discontinuation and Non-adherence to Adjuvant Hormonal Therapy in a Cohort of 8,769 Early Stage Breast Cancer Patients, Hershman et al., Journal of clinical Oncology, vol. 26, No. 27, Sep. 20, 2010, all of which are hereby incorporated by reference for all purposes).

Accordingly, there is a need to develop compositions and formulations containing SERMs, SERDs and AI for the treatment and prevention of breast and reproductive tract disorders that have high potency and efficacy and reduced toxicity profile. Local, effective, easy to administer diagnostic tests and chemotherapy would obviate the side effects of systemic treatment and could produce higher levels of treatment compliance with drugs with improved efficacy.

### SUMMARY OF THE INVENTION

In various aspects, the present disclosure provides pharmaceutical compositions for the treatment of subjects in risk for or having a breast disorder or an estrogen-related disorder, the composition comprising: at least one therapeutic agent; a fatty acid mixture comprising at least one omega-3 fatty acid; and at least one vitamin D compound; wherein the composition is capable of being delivered locally to a tissue.

In various aspects, the present disclosure provides pharmaceutical compositions for the treatment of subject in risk for or having a breast disorder or an estrogen-related disorder, the composition comprising: 0.01 g to 15 g of at least one therapeutic agent; 1 g to 10 g of the fatty acid mixture comprising at least one omega-3 fatty acid; 10 IU to 6000 IU of the at least one vitamin D compound; and a fish oil (qs) to 100 g; wherein the composition is capable of being delivered locally to a tissue.

In various aspects, the present disclosure provides pharmaceutical compositions for treatment of subjects in risk for or having a breast disorder or an estrogen-related disorder comprising: 0.01% to 15% of a SERM, a SERD, an AI or a combination thereof, or pharmaceutically acceptable salts thereof; 10% to 90% of a fatty acid mixture comprising at least one omega-3 fatty acid; 10 IU to 6000 IU of at least one vitamin D compound or pharmaceutically acceptable salts thereof; and 10% to 90% vehicle; wherein the composition is capable of being delivered locally to a tissue.

In various aspects, the present disclosure provides oral pharmaceutical compositions for treatment of subjects in risk for or having a breast disorder or an estrogen-related disorder comprising: at least one therapeutic agent; a fatty acid mixture comprising at least one omega-3 fatty acid; and at least one vitamin D compound.

In various aspects, the present disclosure provides capsules for oral delivery, the capsule comprising: a shell comprising 0.1 mg to 500 mg of a SERM, a SERD, an AI, or a combination thereof; and a fill phase comprising (a) 20% to 60% of a fatty acid mixture comprising at least one omega-3 fatty acid triglyceride or phospholipid; and (b) 10 IU to 6000 IU of at least one vitamin D compound.

In various aspects, the present disclosure provides soft gelatin capsules comprising: a shell comprising 0.5 mg or 1 mg of anastrozole; a fill phase comprising (a) 60% of a fatty acid mixture comprising at least 99% EPA triglyceride or phospholipid; and (b) 400 IU of cholecalciferol; and a sufficient amount of fish oil.

In various aspects, the present disclosure provides methods of preparing pharmaceutical compositions, comprising mixing: at least one therapeutic agent; a fatty acid mixture comprising at least one omega-3 fatty acid; at least one vitamin D compound; optionally, an excipient; and optionally, at least one additional medication.

In various aspects, the present disclosure provides methods preparing pharmaceutical compositions, comprising the steps of: providing an amount of at least one therapeutic agent; providing an amount of a fatty acid mixture comprising at least one omega-3 fatty acid; providing an amount of at least one vitamin D compound; providing at least one excipient; combining the fatty acid mixture comprising at least one omega-3 fatty acid, the at least one vitamin D compound, and the at least one excipient, thereby forming a fill phase; and encapsulating the fill phase in a shell, wherein the at least one therapeutic agent is comprised in the fill phase or the shell or both.

In various aspects, the present disclosure provides methods preparing pharmaceutical compositions, comprising the steps of: providing an amount to a fatty acid oil mixture comprising EPA and DHA at a weight ratio ranging from 1:10 to 10:1; providing at least one vitamin D compound in the fatty acid oil mixture comprising the EPA and DHA; encapsulating the fatty acid oil mixture in a shell; providing the shell with a coating; and providing an amount of anastrozole in the coating of the shell.

In various aspects, the present disclosure provides methods for treatment of subjects at risk for or having a breast disorder or an estrogen-related disorder, the method comprising administering to the subject a pharmaceutical composition comprising: at least one therapeutic agent; a fatty acid mixture comprising at least one omega-3 fatty acid; and at least one vitamin D compound.

In various aspects, the present disclosure provides a method for treatment of a subject at risk for or having a breast disorder or an estrogen-related disorder, the method comprising administering to the subject a pharmaceutical composition comprising: 0.01 g to 15 g of at least one therapeutic agent; 1 g to 10 g of the fatty acid mixture comprising at least one omega-3 fatty acid; 10 IU to 6000 IU of the at least one vitamin D compound; and a fish oil (qs) to 100 g; wherein the composition is capable of being delivered locally to a tissue.

In various aspects, the present disclosure provides methods for treatment of subjects at risk for or having a breast disorder or an estrogen-related disorder, the method comprising administering to the subject a pharmaceutical composition comprising: 0.01% to 15% of a SERM, a SERD, an AI or a combination thereof, or pharmaceutically acceptable salts thereof; 10% to 90% of a fatty acid mixture comprising at least one omega-3 fatty acid; 10 IU to 6000 IU of at least one vitamin D compound or pharmaceutically acceptable salts thereof; and 10% to 90% vehicle.

In various aspects, the present disclosure provides methods for treatment of subjects at risk for or having a breast disorder or an estrogen-related disorder, the method comprising for administering to the subject an oral pharmaceutical composition comprising: at least one therapeutic agent; a fatty acid mixture comprising at least one omega-3 fatty acid; and at least one vitamin D compound.

In various aspects, the present disclosure provides methods for treatment of subjects at risk for or having a breast disorder or an estrogen-related disorder, the method comprising: collecting a NAF sample from the subject; testing the NAF sample using a testing method; determining subject's breast condition; based on subject's breast condition, administering to the subject an amount of a pharmaceutical composition comprising (a) at least one therapeutic agent; (b) a fatty acid mixture comprising at least one omega-3 fatty acid; and (c) at least one vitamin D compound.

In various aspects, the present disclosure provides methods for treatment of subjects at risk for or having a breast disorder or an estrogen-related disorder, the method comprising: collecting a NAF sample from the subject; providing at least one cell from the NAF sample; conducting a whole-genome sequencing of the cell; determining the subject's risk for, presence or reoccurrence of breast disorder or estrogen-related disorder; and administering a therapeutically effective amount of a pharmaceutical composition; wherein the composition comprises at least one therapeutic agent, a fatty acid mixture comprising at least one omega-3 fatty acid, and at least one vitamin D compound.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Aspects of the present invention include methods of treatment, such as methods of treating an estrogen-related disorder, such as breast disorder, methods of delivering an active pharmaceutical agent from a device or a depot in a therapeutically effective amount, such as delivering 4-hdyroxytamoxifen, desmethyltamoxifen, fulvestrant or anastrozole to a subject with decreased side effects and increased bioavailability, reservoir-based drug-delivery compositions; oral, transdermal and parenteral delivery systems and kits for delivery of such systems.

### I. Definitions

As used herein, the terms “a,” “an,” and “the” include plural reference unless the context dictates otherwise.

As used herein, the terms “active pharmaceutical ingredient”, “active ingredient”, “API,” “drug,” “active,” “actives” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present invention is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, or prevention of disease in man or other animals.

As used herein, the term “tamoxifen” refers to (Z)-2-[4-(1,2-Diphenyl-1-butenyl)phenoxy]-N,N-dimethylethanamine.

As used herein, the term “4-hydroxytamoxifen” refers to 4-[(Z)-1-[4-[2-(dimethylamino)ethoxy]phenyl]-2-phenylbut-1-enyl]phenol, and constitutes an active metabolite of tamoxifen.

As used herein, the term “endoxifen” refers to 4-hydroxy-N-desmethyl-tamoxifen and constitutes a secondary metabolite of tamoxifen.

As used herein, the term “droloxifene” refers to 3-[(1E)-1-[4-[2-(dimethylamino)ethoxy]phenyl]-2-phenyl-1-butenyl]phenol.

As used herein, the term “clomifene” refers to 2-[4-(2-Chloro-1,2-diphenylethenyl)phenoxy]-N,N-dimethylethanamine.

As used herein, the term “raloxifene” refers to [6-Hydroxy-2-(4-hydroxyphenyl)benzo[b]thien-3-yl][4-[2-(1-piperidinyl)eth-oxy]-phenyl]methanone.

As used herein, the term “toremifene” refers to 2-[4-(1Z)-4-Chloro-1,2-diphenyl-1-butenyl)phenoxy]-N,N-dimethylethanamine.

As used herein, the term “fulvestrant” refers to 7α-[9-(4,4,5,5,5-Pentafluoropentylsulphhinyl)nonyl]oestra-1,3,-5 (10)-triene-3,17β-diol, (7α,17β)-7-{9-[(4,4,5,5,5pentafluoropentyl)sulfinyl]nonyl}estra-1,3,5(10)-triene-3,17-diol or ICI 182,780.

As used herein, the term “anastrozole” refers to 2,2′-[5-(1H-1,2,4-triazol-1-ylmethyl)-1,3-phenylene]bis(2-methylpropanenitrile).

As used herein, the term “pharmaceutically acceptable” means approved by a regulatory agency, e.g., of the U.S. Federal or state government or listed in the U.S. pharmacopeia or other generally recognized pharmacopeia for use in animals, and more particularly in humans.

As used herein. The term “pharmaceutically acceptable salts” means any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the pharmaceutically active compounds of the present invention may be derived from inorganic or organic acids and bases. Examples of acids include, without limitations, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benesulfonic acid and the like. Other acids such as oxalic acid, while not in themselves pharmaceutically acceptable, may be employed in the preparation of salts useful as intermediates in obtaining the pharmaceutically active compounds used as therapeutic agents of the invention and their pharmaceutically acceptable acid addition salts.

Example of bases include, without any limitations, alkali metal (e.g., sodium), hydroxides, alkaline earth metal (e.g., magnesium) hydroxides, ammonia, and compounds of formula NW₄⁺, wherein W is C₁₋₄ alkyl, and the like.

Examples of salts include, but are not limited to, acetate, adipate, alginate, aspartate, benzoate, benzene-sulfonate, bisulfate, butyrate, citrate, camphorate, camphor-sulfonate, cyclopentaneproprionate, digluconate, dodecylsulphate, ethanesulfonate, umarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Each API and excipient used herein may be discussed interchangeably with respect to its chemical formula, chemical name, abbreviation, etc. For example, HPMC may be used interchangeably for hydroxypropyl methyl cellulose. Additionally, each polymer is described herein, unless designated otherwise, includes homopolymers, copolymers, terpolymers, and the like.

As used herein and in claims, the term “amount,” “effective amount,” and “therapeutically effective amount” may be used interchangeably and is used to describe an amount of the pharmaceutical composition that treats, totally or partially, the progression of a disorder or alleviates, at least partially, one or more symptoms of the disorder. A therapeutically effective amount can also be an amount that is prophylactically effective. The amount that is therapeutically effectively will depend upon the patient's size and gender, the disorder or condition to be treated, the severity of the condition and the result sought. For a given patient and condition, a therapeutically effective amount can be determined by methods known to those skilled in the art.

As used herein, the term “analog” means a chemical compound that is structurally similar to another.

As used herein, the term “dosage form” means the form in which the drug is delivered to the patient. The dosage form means the composition delivered to a subject in any form suitable for parenteral, topical, oral, transdermal, intraductal delivery.

As used herein, the term “composition” and “preparation” may be used interchangeably unless the context suggests otherwise.

The terms “subject,” “patient,” and “individual,” may be used interchangeably herein and refer to a mammalian, such as a human.

As used herein, and in the claims, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of:”

It is specifically understood that any numerical value cited herein includes all values from the lower value to the upper value, i.e., all possible combination of numerical values between the lowest value and the highest value enumerated are to be considered to be expressly stated in this application. For example, if a concentration range or beneficial range is stated as 1% to 50%, it is intended that values such as 2% to 40%, 10% to 30%, or 1% to 3% etc., are expressly enumerated in this specification. As another example, a stated concentration of 20% is intended to include values from 19.5% to 20.5%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

As used herein, and in the claims, the terms “treat,” “treating,” “treatment” as used herein may be used interchangeably and means that a pharmaceutically effective amount of a composition would be administered, which will inhibit, or at least partially arrest or partially prevent or suppress estrogen or inflammation, or reduce a symptom, delay onset, or inhibit recurrence of a breast disorder or an estrogen-related disorder.

For example, the treatment may include a treatment that can suppress or delay the recurrence of breast disorder. The treatment is particularly effective in that once a day dose, such as a transdermal dose or intraductal dose, is administered to a subject, the subject will continue to receive a therapeutically effect dose for the intended duration of the dose (e.g., a week, a month, 6 months or even a year). The treatment is especially beneficial for younger (e.g., <40 years) and older (e.g., >75 years) women who are at a heightened risk for non-compliance and also for those who may be at a greater risk for development or recurrence of breast cancer. The treatment is particularly beneficial for prevention of breast disorder in women who have at least two (2) family members with a history of breast cancer.

As used herein, the term “hydrophilic” describes that something “likes water” i.e., a hydrophilic molecule or portion of a molecule is one that typically is electrically polarized and capable of forming hydrogen bonds with water molecules, enabling it dissolve more readily in water than in oil or other “non-polar” solvents.

As used herein, the term “hydrophobic” denotes a compound tending to be electrically and neutral and thus preferring other nonpolar solvents or molecular environments. In the present context, the term “amphiphilic” describes a molecule (as a surfactant) having a polar water-soluble group attached to a water insoluble hydrocarbon chain. Thus, one end of the molecule is hydrophilic (polar) and the other is hydrophobic (non-polar).

As used herein, the term “vehicle” means any solvent or carrier fluid in a pharmaceutical product that has no pharmacological role. For example, water is a vehicle for xilocaine, and polypropylene glycol is the vehicle for many antibiotics.

As used herein, the term “penetration enhancer” or “permeation enhancer” means an agent known to accelerate the delivery of the drug through the skin. These agents can also be referred to as accelerants, adjuvants, and absorption promoters, and are collectively referred to herein as “enhancers.” Penetration enhancers according to the present invention are introduced in a non-irritating and/or non-sensitizing amount.

As used herein, the expression “non-irritating” and/or “non-sensitizing” refers to amounts and/or types of excipients used which the skilled person in the art would consider to be well-tolerated by the human skin, i.e., dermatologically acceptable. Using common general knowledge, the skilled person can determine non-irritating and/or non sensitizing amounts of an excipient, for example, a penetration enhancer or an adhesive. In some embodiments, the non-irritating and/or non-sensitizing amount results in no detectable or sustained dermal adverse reaction (e.g., itching, reddening, burning sensation), or results in only a minimal reaction that is generally deemed to be acceptable by patients and health care providers.

As used herein, the term “hydroalcoholic” refers to a substance or a composition that comprises both water and alcohol.

As used herein, the term “gelling agent” means a composition or a substance that when dissolved suspended or dispersed in a fluid (for e.g., an aqueous fluid such as water or a buffer solution) forms a gelatinous semi-solid (e.g., a lubricant gel). Examples of gelling agents include but are not limited to hydroxyethyl cellulose, hydroxymethyl cellulose, hydroxypropyl guar, methyl cellulose, ethyl cellulose, hydroFulose, carbomers, alginates, gelatin, and poloxomer.

As used herein, the term “excipient” refers to an inactive ingredient (i.e., not pharmaceutically active) added to a preparation of an active ingredient. The gelling agent and penetration enhancers for example can generally be referred to as excipients.

As used herein, the terms “controlled-release” refers to the release of a therapeutic agent in a way that deviates from immediate release. As used herein, the terms “sustained-release” and “extended-release” are used interchangeably and refer to the release of the administered therapeutic agent over a longer period of time than a comparable immediate release formulation, resulting in levels of the therapeutic agent or an API in the affected tissues elevated over baseline for a longer period of time than for a comparable immediate-release formulation. The foregoing terms optionally include delayed release characteristics. For example, a delayed release type of controlled-release formulation will be characterized by C at a time greater than C for an immediate release formulation. For example, the released of a therapeutic agent such as 4-OHT will preferably be at such a rate that total ductal, blood or serum levels of 4-OHT are maintained or elevated above the pre-dosing levels for an extended period of time, e.g., 4 hours to 24 hours or even longer.

As used herein, the term “bioavailability” denotes the degree to which a drug or other substances becomes available to the target tissue after administration. As used herein, the term “bioequivalency” denotes a scientific basis on which generic and brand name drugs are compared to one another. For example, drugs are bioequivalent if they enter circulation at the same rate when given in similar doses under similar conditions. Parameters often used in bioequivalence studies are tₘₐₓ, Cₘₐₓ, AUC_{o-infinity}, AUCₒ₋ᵣ. Other relevant parameters may be W50, W75 and/or MRT. Accordingly, at least one of these parameters may be applied when determining whether bioequivalence is present.

As used herein, the term “NAF” denotes breast duct fluids obtained by nipple aspiration and/or ductal lavage.

As used herein, the terms “condition,” “disorder,” and “disease,” may be used interchangeably.

As used herein, the terms “estrogen-related disorder” and “estrogen-receptor disorder” may be used interchangeably and includes, without limitation, disorders with high estrogen levels or normal estrogen levels that need to be reduced, disorders with estrogen-receptor positive (ER+) and/or progesterone-receptor positive (PR+) disorders, for example, breast disorders, endometriosis, uterine fibroids (also called leiomyomas) etc.

As used herein, “breast disorder” means any aberration or a constellation of aberrations in the breast. Such aberration may be proliferative or non-proliferative. Breast disorders include benign lesions of the breast (hyperplasia) and breast cancer. Benign breast lesions include, but are not limited to, ductal hyperplasia, lobular hyperplasia, atypical ductal hyperplasia, and atypical lobular hyperplasia.

As used herein, “breast cancer” means any malignant tumor of breast cells. There are several types of breast cancer. Exemplary breast cancers include, but are not limited to, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, ER-negative (ER−) breast cancer, HER2+ breast cancer, triple negative breast cancer (TNBC), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, or micropapillary carcinoma. A single breast cancer tumor can be a combination of these types or be a mixture of invasive and in situ cancer.

Ductal hyperplasia is a hyperplasia of a breast duct, not accompanied by histomorphologic abnormalities. Ductal hyperplasia is not usually considered predictive of a predisposition for breast cancer.

Lobular hyperplasia is a hyperplasia of a breast lobule, not accompanied by histomorphologic abnormalities. Lobular hyperplasia is not usually considered predictive of a predisposition for breast cancer.

Atypical ductal hyperplasia (ADH) is a benign lesion of the breast characterized by hyperplasia of at least one breast duct and histomorphologic abnormalities. While not cancerous, ADH may be indicative of a predisposition for breast cancer. ADH may be excised by lumpectomy.

Atypical lobular hyperplasia (ALH) is a benign lesion of the breast characterized by a hyperplasia of a breast lobule and histomorphologic abnormalities. While not cancerous, ALH may be indicative of a predisposition for breast cancer. ALH may be excised by lumpectomy.

Ductal carcinoma in situ (DCIS) is the most common non-invasive breast cancer. It involves the cell lining the breast ducts. In DCIS, the cells have not spread beyond the walls of the duct into the surrounding breast tissue. About 1 in 5 new breast cancer cases will be DCIS.

Lobular carcinoma in situ (LCIS) is a pre-cancerous neoplasia. It may be indicative of a predisposition for invasive cancer. LCIS only accounts for about 15% of the in situ (ductal or lobular) breast cancers. LCIS is often treated with SERMs, predominantly tamoxifen.

Invasive Ductal Carcinoma (IDC) is the most invasive breast cancer. As the name applies, it is a carcinoma that begins in the breast ducts and then invades the surrounding fatty tissue. About 8 to 10 invasive breast cancers are infiltrating ductal carcinomas. IDC is often treated by surgery to excise the cancerous tissue, and radiation therapy. In addition, chemotherapy combined with immunotherapy (e.g., tamoxifen and tratuzumab) is often used to treat DC. If the tumor is larger than 4 cm, then a radial mastectomy may be performed.

Invasive Lobular Carcinoma (ILC) is a cancer that develops in the lobules of the breast and has invaded the surrounding tissue. About 1 in 10 invasive breast cancer is an ILC. ILC is treated by surgery to excise the cancerous tissue, and radiation therapy. In addition, chemotherapy and immunotherapy combination (e.g., tamoxifen and tratuzumab) is often used as an adjuvant therapy to treat ILC.

Inflammatory breast cancer accounts for about 1% to 3% of all breast cancers. In inflammatory breast cancer, cancer cells block lymph vessels in the skin resulting in the beast turning red and feeling warm. The affected breast may become larger or firmer, tender, or itchy. Inflammatory breast cancer is treated with chemotherapy, immunotherapy, radiation therapy and in some case, surgery.

ER+ breast cancer is characterized by the presence of estrogen receptors on the surface of the cancerous cells. Growth of ER+ cancer cells is associated with the availability of estrogen. Treatment options for ER+ breast cancer include chemotherapeutic agents that block estrogen (e.g., tamoxifen).

HER2+ breast cancers are characterized by an excess of HER2 on cell surface of the cancerous cells. HER2+ cancer is often treated with trastuzumab in combination with additional chemotherapeutic agents.

Triple Negative Breast Cancer (TNBC) is a breast cancer characterized by cell which lack estrogen receptors and progesterone receptors, and do not have an excess of the HER2 protein on their surface. TNBC are often more invasive than other breast cancers. Because the tumor cells lack estrogen and progesterone receptors, hormone therapy (e.g., tamoxifen) by itself is not effective. Additionally, as the cells lack the HER2 protein, drugs that target HER2 (e.g., trastuzumab) are ineffective.

### II. Disorders

The compositions and formulations disclosed herein are useful for the treatment of subjects in risk for or having any estrogen-related disorder or a breast disorder. In one aspect, the compositions and formulations disclosed herein may be used to treat subjects at risk for or having an estrogen-related disorder. In some embodiments, subjects have high estrogen levels or normal estrogen levels that need to be reduced. In other embodiments, the subjects have estrogen-receptor positive (ER+) and/or progesterone-receptor positive (PR+) disorders, for example, breast disorders, endometriosis, uterine fibroids (also called leiomyomas), etc. In yet other embodiments, the subjects have estrogen-receptor positive (ER+) and/or HER2 positive (HER2+) disorders.

In some embodiments, the compositions disclosed herein may be used to treat breast disorders, including, without limitation, benign lesions of a breast, and breast cancer. In some preferred embodiments, the breast disorder is selected from a group consisting of ductal hyperplasia, lobular hyperplasia, atypical ductal hyperplasia, atypical lobular hyperplasia, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, ER-negative (ER−) breast cancer, HER2+ breast cancer, triple negative breast cancer (TNBC), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, and micropapillary carcinoma. In a preferred embodiment, the disorder is a combination of the foregoing breast disorders. In a more preferred embodiment, the breast disorder is a mixture of invasive and in situ cancer. In one aspect, the breast disorder is a malignant tumor of breast cells. In another aspect, the breast disorder includes conditions where the breast tissues express increased levels of HER2. In a preferred embodiment, the breast disorder is hyperplasia.

Without being bound by a particular theory of operation, precancerous hyperplasia of the breast is “driven” by a number of processes. A significant process is the contribution of stimulation of the estrogen/progesterone hormonal axis. Each menstrual cycle, during the proliferative phase and especially week two of the cycle, blood levels of estrogen increase significantly, driving ductal cell division and growth. Following ovulation, if fertilization does not occur, there is involution of the ductal and lobular changes and return to quiescence until the next cycle. Estrogen from systemic sources, mostly from ovaries, as well as local synthesis within the breast from the action of aromatase on testosterone contribute to the growth. A second major stimulation is the generalized effect of a pro-inflammatory environment. This has been considered by some to be effect of stromal effects on the ductal epithelium. A third stimulation involves the role of “metabolic” drivers, such as glucose driven metabolism and high mitochondrial activity in the process. Fourth, a general role for vitamin D as a driver in the regulation of a wide range of cellular mechanisms central to cancer development, such as apoptosis (cell death), cell proliferation, differentiation, angiogenesis and metastasis has been suggested. Finally, HER2 stimulation and oncogene and tumor promoter activation can contribute to either inducing hyperplasia or sustaining it.

Given the drivers of precancerous hyperplasia, certain classes of effectors may be used to prevent or reverse the hyperplasia, and treat estrogen-related disorders and breast disorders. For example, SERMs, SERDs, and AIs may block the effects of the estrogen surge. Given further the increased inflammation, heart disease, bone loss and osteoporosis resulting from the current adjuvant systemic therapy with tamoxifen, raloxifen, etc., pharmaceutical compositions of the present invention combining these SERMs, SERDs, and/or Ms with omega-3 fatty acids and vitamin D may provide a solution to the unmet need for new pharmaceutical compositions, formulations and treatment methods that prevent or treat subjects at risk for development or recurrence of ER+ disorder and/or breast disorder, reduce these toxic effects, particularly inflammation, cardiac health, bone loss and osteoporosis and/or enhance patient compliance.

### III. Pharmaceutical Compositions

The present invention provides pharmaceutical compositions for the prevention and/or treatment of a subject in risk for or having an estrogen-related disorder or a breast disorder that reduce the risk of developing, progressing or recurring ER+ disorders and/or breast disorders and also reduce systemic effects of the SERDs, SERMs and/or AI. The compositions disclosed herein are capable of being delivered locally at rates of delivery ranging from rapid delivery to slow or sustained delivery with little to no systemic blood concentrations of therapeutic agents.

A. Active Pharmaceutical Agents

In one aspect of the present invention, the pharmaceutical compositions comprise: at least one therapeutic agent; a fatty acid mixture comprising at least one omega-3 fatty acid; and at least one vitamin D compound. Accordingly, the pharmaceutical composition of the present invention comprises at least three active pharmaceutical ingredients: (i) at least one therapeutic agent; (ii) a fatty acid mixture comprising at least one omega-3 fatty acid; and (iii) at least one vitamin D compound. In some embodiments, the pharmaceutical compositions comprise more than three active pharmaceutical ingredients, for example, the compositions may comprise an additional medication such as a cytotoxic agent such as trastuzumab or a Selective Androgen Receptor Modulator (SARM).

In some embodiments, the pharmaceutical compositions comprise: at least one therapeutic agent; a fatty acid mixture comprising at least one omega-3 fatty acid; and at least one vitamin D compound; wherein the pharmaceutical compositions are capable of being delivered locally to a tissue. In some embodiments, the at least one therapeutic agent comprises a SERM, a SERD, an AI, or a combination thereof, and pharmaceutically acceptable salts thereof. In some embodiments, the SERM is selected from the group consisting of tamoxifen, cis-tamoxifen, 4-OHT, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923. In some embodiments, the SERM comprises 4-OHT, desmethyltamoxifen, or endoxifen. In some embodiments, the SERD comprises a fulvestrant, ARN-810, or CH4986399. In some embodiments, the AI is selected from the group consisting of anastrozole, exemestane and letrozole. In some embodiments, the at least one therapeutic agent is between 0.01% to 15% by weight of the composition. In some embodiments, the at least one therapeutic agent is between 0.01% to 15% by weight of the composition. In some embodiments, the at least one omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the omega-3 fatty acid is a triglyceride or a phospholipid. In some embodiments, the fatty acid mixture comprising at least one omega-3 fatty acid is between 10% to 90% by weight of the composition. In some embodiments, the fatty acid mixture comprises from 400 mg/g to 600 mg/g of the at least one omega-3 fatty acid. In some embodiments, the fatty acid mixture comprises a plurality of omega-3 fatty acids. wherein the fatty mixture comprises a mixture of EPA and DHA. wherein the fatty acid mixture is a fatty acid oil mixture. wherein the fatty acid oil mixture is derived from at least one oil selected from a group consisting of a marine oil, a plant-based oil, an algae oil, a microbial oil, and a combination thereof. wherein the marine oil is a fish oil. the fatty acid mixture is an emulsion. In still further embodiments, the emulsion is an alcohol-in oil emulsion, an oil-in-alcohol emulsion, oil-in-water emulsion, water-in-oil emulsion, water-in-oil-in-water emulsion, or an oil/alcohol/water emulsion. In some embodiments, the at least one vitamin D compound is selected from the group consisting of calciferol, cholecalciferol, ergocalciferol, vitamin D metabolites, 25-hydroxyvitamin D3, 25-hydroxyvitamin D2, 25(OH)D, 1,25(OH)(2)D, 25-hydroxyvitamin D4, 25-hydroxyvitamin D5, 25-hydroxyvitamin D7, 1-alpha-25-hydroxyvitamin D3, 1-alpha-25-hydroxyvitamin D2, 1-alpha-25-hydroxyvitamin D4,1,25-dihydroxy-19-nor-vitamin D2, 1-alphahydroxyvitamin D3, vitamin D analogs, and a combination thereof. In some embodiments, the at least one vitamin D compound is cholecalciferol. In some embodiments, the at least one vitamin D compound is partly or wholly dissolved, dispersed, or suspended in the fatty acid mixture comprising the at least one omega-3 fatty acid. In some embodiments, the at least one therapeutic agent and the at least one vitamin D compound are partly or wholly dissolved, dispersed or suspended in the fatty acid mixture comprising the at least one omega-3 fatty acid. In further embodiments, the at least one vitamin D compound has an activity ranging between 10 IU-6000 IU.

In some embodiments, the pharmaceutical compositions further comprise an excipient. In some embodiments, the composition is formulated in a gel, a solution, a lotion, an ointment, a cream, or an emulsion. wherein the gel comprises a vehicle, co-solvent, a stabilizing agent, a neutralization agent, a permeation enhancer, an absorption enhancer, a surfactant, a gelling agent, a polymer, a co-polymer, a cross-linking agent, an antioxidant, a moisturizer, an antimicrobial, a preservative, or a combination thereof. In some embodiments, the vehicle is an oily vehicle. In some embodiments, the oily vehicle is fish oil. In some embodiments, the gelling agent is HPMC, CMC, Carbopol or polyacrylic acid. In some embodiments, the permeation enhancer is an ether, a sulfoxide, a poloxomer, a pyrrolidone, an azone, or a fatty alcohol. In some embodiments, the surfactant is SDS, cetrimide, Capmul, Cremaphor, or Tween 85. In some embodiments, the antioxidant is alpha-tocopherol, BHA, BHT, ascorbic acid and pharmaceutically acceptable salts and esters thereof, propyl gallate, citric acid and pharmaceutically acceptable salts thereof, malic acid and pharmaceutically acceptable slats thereof, and sulfite salts and mixtures thereof.

In some embodiments, the pharmaceutical compositions further comprise at least one additional medication. In some embodiments, the at least one additional medication is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof. wherein the at least one additional medication is trastuzumab.

In some embodiments, the pharmaceutical compositions are delivered using a transdermal, a transpapillary, or an intraductal device. In some embodiments, the transdermal device is selected from the group consisting of an applicator, a patch, a tape, sheet, a dressing, a spray device and an aerosolizer. In some embodiments, the patch for transdermal delivery comprises: a backing layer; and an adhesive layer having a skin-contacting adhesive surface; wherein the adhesive layer comprises a drug reservoir comprising (a) at least one therapeutic agent, (b) a fatty acid mixture comprising at least one omega-3 fatty acid, and (c) at least one vitamin D compound sufficient to treat a breast disorder or estrogen-related disorder for at least three days. In some embodiments, the patch for transdermal delivery comprises: a backing layer; a drug reservoir disposed on a first layer; and a skin-contacting second layer comprising a pressure sensitive adhesive layer; wherein the second layer is attached to a surface of the first layer opposed to a surface in contact with the backing layer, wherein the second layer is a rate-controlling layer and wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days. In some embodiments, the patch for transdermal delivery comprises: a backing layer; a drug reservoir disposed on a first layer; a second layer comprising a rate-controlling membrane, the membrane being attached to a surface of the first layer opposed to a surface in contact with the backing layer; and a skin-contacting third layer comprising a pressure sensitive adhesive attached to a surface of the membrane that is opposed to the surface of the rate-controlling membrane in contact with the first layer; wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days. In some embodiments, the breast disorder is a proliferative breast disease, a breast cancer, a breast scarring, or an increase in breast density. In some embodiments, the breast cancer is ductal carcinoma in situ (DCIS), microinvasive breast carcinoma (MIC), lobular carcinoma in situ (LCIS), invasive (or infiltrating) lobular carcinoma (ILC), invasive ductal carcinoma (DC), or inflammatory breast cancer, ER-positive breast cancer, ER-negative breast cancer, triple negative breast cancer (TNBC), adenoid cystic (adenocystic) carcinoma, law-grade adenosquamatous carcinoma, medullary carcinoma, mucinous (or colloid) carcinoma, papillary carcinoma, tubular carcinoma, metaplastic carcinoma, and micropapillary carcinoma. In some embodiments, the breast disorder is a proliferative breast disease, a breast cancer, a breast scarring, or an increase in breast density. the proliferative beast disease is a mild hyperplasia, hyperplasia of the usual type, atypical ductal hyperplasia, and atypical lobular hyperplasia. In some embodiments, the breast disorder is a proliferative breast disease, a breast cancer, a breast scarring, or an increase in breast density. breast cancer is ER+ metastatic breast cancer, ER+ refractory breast cancer, AR+/ER+ breast cancer, AR+/ER+ refractory breast cancer, AR+/ER+ metastatic breast cancer, and triple positive breast cancer.

In some embodiments, the pharmaceutical compositions comprise: 0.01 g to 15 g of at least one therapeutic agent; 1 g to 10 g of the fatty acid mixture comprising at least one omega-3 fatty acid; 10 IU to 6000 IU of the at least one vitamin D compound; and a fish oil (qs) to 100 g; wherein the composition is capable of being delivered locally to a tissue.

In some embodiments, the pharmaceutical compositions comprise: 0.01% to 15% of a SERM, a SERD, an AI or a combination thereof, or pharmaceutically acceptable salts thereof; 10% to 90% of a fatty acid mixture comprising at least one omega-3 fatty acid; 10 IU to 6000 IU of at least one vitamin D compound or pharmaceutically acceptable salts thereof; and 10% to 90% vehicle. In some embodiments, the composition is capable of being delivered locally to a tissue. In some embodiments, the composition comprises at least one gelling agent. In some embodiments, the at least one gelling agent is 0.1% to 80% w/w of the composition.

In some embodiments, the SERM may be selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652, ERA-923, and pharmaceutically acceptable salts thereof. In some embodiments, the SERD is selected from a group consisting of fulvestrant, ARN-810, CH4986399, and pharmaceutically acceptable salts thereof. In some embodiments, the AI selected from a group consisting of anastrozole, exemestane, letrozole, and pharmaceutically acceptable salts thereof.

In some embodiments, the pharmaceutical compositions or oral pharmaceutical compositions comprise a first composition comprising the fatty acid mixture comprising the at least one omega-3 fatty acid, a second composition comprising the at least one vitamin D compound, and a third composition comprising the at least one therapeutic agent. In some embodiments, the pharmaceutical compositions or oral pharmaceutical compositions comprise a first composition comprising the fatty acid mixture comprising the at least one omega-3 fatty acid and the at least one vitamin D compound and a second composition comprising the at least one therapeutic agent. In some embodiments, the pharmaceutical compositions or oral pharmaceutical compositions comprise a single composition comprising the fatty acid mixture comprising the at least one omega-3 fatty acid, the at least one vitamin D compound and the at least one therapeutic agent.

In some embodiments, there are provided methods of preparing pharmaceutical compositions, comprising mixing: at least one therapeutic agent; a fatty acid mixture comprising at least one omega-3 fatty acid; at least one vitamin D compound; optionally, an excipient; and optionally, at least one additional medication. In some embodiments, the methods of preparing pharmaceutical compositions comprise formulating pharmaceutical compositions for local delivery.

In some embodiments, methods of preparing pharmaceutical compositions comprise the steps of: providing an amount of at least one therapeutic agent; providing an amount of a fatty acid mixture comprising at least one omega-3 fatty acid; providing an amount of at least one vitamin D compound; providing at least one excipient; combining the fatty acid mixture comprising at least one omega-3 fatty acid, the at least one vitamin D compound, and the at least one excipient, thereby forming a fill phase; and encapsulating the fill phase in a shell, wherein the at least one therapeutic agent is comprised in the fill phase or the shell or both.

In some embodiments, the methods of preparing pharmaceutical compositions comprise the steps of: providing an amount to a fatty acid oil mixture comprising EPA and DHA at a weight ratio ranging from 1:10 to 10:1; providing at least one vitamin D compound in the fatty acid oil mixture comprising the EPA and DHA; encapsulating the fatty acid oil mixture in a shell; providing the shell with a coating; and providing an amount of anastrozole in the coating of the shell.

In some embodiments, the methods of preparing pharmaceutical compositions further comprise providing at least one additional medication in the shell or the fill phase or both.

In some embodiments, the capsule is a hard capsule or a soft capsule. In some embodiments, the capsule shell is prepared using a plate process, a rotary die process, or a reciprocating die process.

i. Therapeutic Agents

The at least one therapeutic agent is selected from a group consisting of a SERM, a SERD, an AI or a combination thereof, and pharmaceutically acceptable salts thereof. When the therapeutic agent is a combination, the therapeutic agent may be any combination or permutation of a SERM, a SERD, or an AI, or pharmaceutically acceptable salts thereof. For example, the therapeutic agent component may be a combination of one or more of a SERD with one or more of a SERM and/or one or more of AI. As another non-limiting example, the therapeutic agent may be a combination of one or more of a SERD and one or more of an AI. One of skill in the art will recognize that the invention is not so limited as to permit a combination only one SERM and SERD or AI, and that all possible combinations fall within the scope of the present invention.

In a preferred embodiment, the at least one therapeutic agent is a SERM or a pharmaceutically acceptable salt thereof. In some embodiments, the at least one therapeutic agent is a SERM selected from the group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923. In a preferred embodiment, the SERM is cis-tamoxifen or a pharmaceutically acceptable salt thereof. In at least one embodiment, the SERM is a tamoxifen metabolite. In a preferred embodiment, the SERM is endoxifen or a pharmaceutically acceptable salt thereof. In yet another embodiment, endoxifen is a mixture of the E-(cis-) and Z- (trans-) isomers of the endoxifen or pharmaceutically acceptable salts thereof. In yet another embodiment, the endoxifen is an E-isomer or a pharmaceutically acceptable salt thereof. In still another embodiment, the endoxifen is a Z-isomer or a pharmaceutically acceptable salt thereof. In another preferred embodiment, the SERM is 4-hydroxytamoxifen (4-OHT) or a pharmaceutically acceptable salt thereof. In yet another embodiment, the 4-OHT is an E-isomer or a pharmaceutically acceptable salt thereof. In still another embodiment, the 4-OHT is a Z-isomer or a pharmaceutically acceptable salt thereof. In a further embodiment, the 4-OHT is a mixture of the E- and Z-isomers of the 4-OHT or pharmaceutically acceptable salts thereof. The E/Z isomer mixture preferably is a stable mixture. The ratio of the E- and the Z-isomers in the mixture may be any suitable ratio. For example, in some embodiments, E:Z isomer may be present at 70:30, 65:35, 60:40, 55:45, or 50:50 in the E/Z mixture. In other embodiments, the Z:E may be present at 70:30, 65:35, 60:40, 55:45, or 50:50 in the Z/E isomer mixtures.

In yet other preferred embodiments, the SERM is desmethyltamoxifen. In another embodiment, the therapeutic agent is a SERD a pharmaceutically acceptable salt thereof. In some preferred embodiments, the SERD is selected from a group consisting of fulvestrant, ARN-810, and CH4986399. In a preferred embodiment, the SERD is fulvestrant or a pharmaceutically acceptable salt thereof. In another embodiment, the therapeutic agent is an AI or a pharmaceutically acceptable salt thereof. In some preferred embodiments, the AI is selected from the group consisting of anastozole (Arimidex™), exemestrane (Arosmasin®), and letrozole (Femara®). In a preferred embodiment, the AI is anastrozole. In another preferred embodiment, the AI is leterozole.

Those skilled in the art will understand that the present invention contemplates that pharmaceutically acceptable salts and equivalents of the therapeutic agents disclosed herein include prodrugs, metabolites, enantiomers, isomers, tautomers, salts, chelates, amides, and derivatives of each SERM, SERD and AI mixtures.

ii. Fatty Acid Mixtures

The compositions disclosed herein offer a way to reduce the side effects observed with the current adjuvant therapy for the treatment of breast cancer. Omega-3 fatty acids are useful in the prevention and treatment of heart disease and inflammation. Accordingly, the compositions of the present invention comprise a fatty acid mixture comprising at least one omega-3 fatty acid. As used herein, the term “fatty acid mixture” includes fatty acids, such as unsaturated (e.g., monounsaturated, polyunsaturated) or saturated fatty acids, as well as pharmaceutically-acceptable esters, free fatty acids, mono-, di-, triglycerides, phospholipids, derivatives, conjugates, precursors, salts and mixtures thereof. In some embodiment, the fatty acid mixture comprises fatty acids, such as omega-3 fatty acids, in a form selected from esters, triglycerides, phospholipids, and free acid form. As used herein, “omega-3 fatty acids” includes natural and synthetic omega-3 fatty acids, as well as pharmaceutically acceptable esters, free acids, mono-, di-, triglycerides, phospholipids, derivatives, conjugates, precursors, salts and mixtures thereof. The omega-3 fatty acid comprised in the fatty acid mixture acts as an active pharmaceutical ingredient (API). In some embodiments, the fatty acid mixture comprising the at least one omega-3 fatty acid is present in a pharmaceutically acceptable amount.

In some embodiments, the at least one omega-3 fatty acid is selected from a group consisting of eicosapentaenoic acid (EPA), docosahexaenoic acid (DHA), alpha-linolenic acid (ALA), hexadecatrienoic acid (HTA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), clupanodonic acid, tetracosapentaenoic acid, tetracosahexaenoic acid (Nisinic acid), and combinations thereof.

In some embodiments, the at least one omega-3 fatty acid is esterified. Non-limiting examples include alkyl esters, methyl esters, and ethyl esters. A preferred embodiment of the at least one omega-3 fatty acid ester is ethyl ester. Another preferred embodiment of the at least one omega-3 fatty acid ester is methyl ester. In another preferred embodiment, the at least one omega-3 fatty acid is in a triglyceride form. The present invention contemplates that triglycerides used herein include mono-, di-, triglycerides and a combination thereof. The present invention also encompasses triglyceride comprising same or different omega-3 acids selected from the group described above. The omega-3 fatty acids of the triglycerides may be short chain, medium chain and long chain fatty acids or a combination thereof. In yet other embodiments, the at least one omega-3 acid is in a phospholipid form.

In one aspect, the fatty acid mixture is an emulsion and comprises emulsified forms of the omega-3 free fatty acid. In some embodiments, the emulsion is an oil-in-alcohol emulsion, an alcohol-in-oil emulsion, an oil-in alcohol emulsion, an oil/alcohol/alcohol emulsion, oil-in-water emulsion, a water-in-oil emulsion, or a water-in-oil-in-water emulsion. In some preferred embodiments, the emulsion is an oil-in-water emulsion. In other preferred embodiments, the emulsion is an oil-in-alcohol emulsion or an oil/alcohol/water emulsion. The emulsions may include emulsifiers that are hydrophilic or lipophilic. In some embodiments, the fatty acid mixture comprising at least one omega-3 acid further comprises at least one surfactant.

In some embodiments, the surfactant may be a hydrophilic surfactant or a lipophilic surfactant or a combination of both. Suitable examples of surfactants include, without limitation, glycerol acetates, glycerol fatty acid esters, acetylated glycerol fatty acid esters, propylene glycol esters, ethylene glycol esters, propylene glycol monocaprylate, mixtures of glycerol and polyethylene glycol esters of long fatty acids, polyethoxylated castor oils, nonylphenol ethoxylates, oleoylmacrogol glycerides, propylene glycol monolaurate, propylene glycol dicaprylate/dicaprate, polyehthylene-polypropylene glycol copolymer, polyoxyethylene-sorbitan-fatty acid esters such as SPAN and Tween, and polyoxyethylene sorbitan monooleate. Surfactant may be chosen from glycerol fatty acid esters such as for example those comprising a fatty acid component of 6-22 carbon atoms, such as glyceryl stearate, glycol stearate, PEG-6/PEG-32/glycol stearate mixture. Suitable acetylated glycerol fatty acid esters include, without limitation, acetylated monoglyclerides, acetylated diglyclerides, and/or mixtures thereof. Capmul® MCM (medium chain mono-, di-glycerides) is a non-limiting example of a commercial glyceride that is suitable for use.

Commercially available hydrophilic surfactants that may be used are supplied under the tradenames, Cremaphor or Etocas and include, without limitation, Cremaphor EL and RH 40 and Etocas 35 and 40, Cremaphor, RH140, polysorbate 60, or Etocas 40.

iii. Vitamin D Compounds

The compositions of the present invention include at least one vitamin D compound.

As used herein, “vitamin D compound” means any vitamin D compound that can act as an active pharmaceutical ingredient and is suitable for prophylactic or therapeutic use or both, and combinations thereof, are contemplated for inclusion in the pharmaceutical composition and formulation described herein. Vitamin D, 25-hydroxyvitamin D, 1, 25-dihydroxyvitamin D, and other metabolites and analogs of vitamin D are also useful as active compounds in pharmaceutical compositions. Specific examples include, but are not limited to, vitamin D₃ (cholechalciferol), vitamin D₂ (ergocalciferol), 25-hydroxyvitamin D3, 25-hydroxyvitamin D2, 25-hydroxyvitamin D4, 25-hydroxyvitamin D5, 25-hydroxyvitamin D7, 1-alpha-25-hydroxyvitamin D3, 1-alpha-25-hydroxyvitamin D2, 1-alpha-25-hydroxyvitamin D4, and vitamin D analogs (including all hydroxyl and dihydroxy forms), including 1,25-dihydroxy-19-nor-vitamin D2, 1-alphahydroxyvitamin D3.

### IV. Formulations

In one aspect, the pharmaceutical compositions disclosed herein are formulated to comprise at least one therapeutic agent, a fatty acid mixture comprising at least one omega-3 fatty acid, at least one vitamin D compound, and optionally, at least one excipient. In another aspect, the pharmaceutical compositions comprise at least one therapeutic agent, at least one omega-3 fatty acid triglyceride or phospholipid, and at least one vitamin D compound, and optionally, at least one excipient.

A. Therapeutic Agents

Typically, a composition may comprise from 0.01% to 15% (w/w) of the SERM, a SERD, an AI or a combination thereof, or a pharmaceutical salt thereof. Where the therapeutic agent in the composition is a combination, the concentration of each therapeutic agent in the composition may range from 0.01% to 15% of the total composition. In some preferred embodiments, a composition may comprise from 0.05% to 12%, from 1% to 10%, from 5% to 10%, from 6% to 8% (w/w) of a SERM, a SERD, an AI or a combination thereof.

In some embodiments, a composition may comprise from 0.01% to 15% (w/w) of a SERM or a pharmaceutically acceptable salt thereof. In some embodiments, a composition of the invention may comprise 0.01%, 0.05%, 0.01%, 0.2%, 0.3%, 0.4%, 0.5%, 0.6% 0.7%, 0.8%, 0.9%, 1%, 1.5%, 2%, 2.5%, or 3% of an SERM. In some embodiments, the SERM is selected from the group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923. In some embodiments, a composition may comprise from 0.01% to 15% (w/w) of cis-tamoxifen. In some preferred embodiments, a composition may comprise from 0.05% to 12%, from 1% to 10%, from 5% to 10%, from 6% to 8% (w/w) of cis-tamoxifen. Typically, a composition of the invention may comprise 0.01% to 15% (w/w) of 4-OHT. In some preferred embodiments, a composition may comprise from 0.05% to 12%, from 1% to 10%, from 5% to 10%, from 6% to 8% (w/w) of 4-OHT. In some embodiments, a composition may comprise 0.01% to 15% (w/w) of endoxifen. In some preferred embodiments, a composition may comprise from 0.05% to 12%, from 1% to 10%, from 5% to 10%, from 6% to 8% (w/w) of endoxifen. In some embodiments, a composition may comprise 0.01% to 15% (w/w) of desmethyltamoxifen. In some preferred embodiments, a composition may comprise from 0.05% to 12%, from 1% to 10%, from 5% to 10%, from 6% to 8% (w/w) of desmethyltamoxifen. In other embodiments, a composition of the invention may comprise 0.01%, 0.05%, 0.01%, 0.2%, 0.3%, 0.4%, 0.5%, 0.6% 0.7%, 0.8%, 0.9%, 1%, 1.5%, 2%, 2.5%, or 3% of an SERD. In some embodiments, a composition may comprise from 0.01% to 15% (w/w) of a SERD or a pharmaceutically acceptable salt thereof. In some preferred embodiments, a composition may comprise from 0.05% to 12%, from 1% to 10%, from 5% to 10%, from 6% to 8% (w/w) of a SERD or a pharmaceutically acceptable salt thereof. In some embodiments, a composition may comprise 0.01% to 15% (w/w) of fulvestrant or a pharmaceutically acceptable salt thereof. In some preferred embodiments, a composition may comprise from 0.05% to 12%, from 1% to 10%, from 5% to 10%, from 6% to 8% (w/w) of fulvestrant.

In some embodiments, a composition may comprise from 0.01% to 15% (w/w) of an AI or a pharmaceutically acceptable salt thereof. In yet other embodiments, a composition of the invention may comprise 0.01%, 0.05%, 0.01%, 0.2%, 0.3%, 0.4%, 0.5%, 0.6% 0.7%, 0.8%, 0.9%, 1%, 1.5%, 2%, 2.5%, or 3% of an AI. In some embodiments, typically a composition may comprise 0.01% to 15% (w/w) of an AI. In some preferred embodiments, the composition may comprise from 0.05% to 12%, from 1% to 10%, from 5% to 10%, from 6% to 8% (w/w) of an AI. In some embodiments, a composition may comprise 0.01% to 15% (w/w) of AI selected from a group consisting of anastrozole, exemestane, and letrozole. In some preferred embodiments, the composition may comprise from 0.05% to 12%, from 1% to 10%, from 5% to 10%, from 6% to 8% (w/w) of anastrozole. In some embodiments, a composition may comprise 0.01% to 15% (w/w) of letrozole. In some preferred embodiments, the composition may comprise from 0.05% to 12%, from 1% to 10%, from 5% to 10%, from 6% to 8% (w/w) of letrozole.

In some embodiments, the formulations deliver 0.1 mg to 500 mg cis-tamoxifen, or the equivalent thereof, to a subject per dosage unit. In another embodiment of the present invention, the formulation delivers from 5 mg to 150 mg cis-tamoxifen, or the equivalent thereof, to a subject per dosage unit. In yet another embodiment, the formulations deliver from 25 mg to 100 mg cis-tamoxifen, or the equivalent thereof, to a subject per dosage unit. In another embodiment of the present invention, the formulation delivers 50 mg to 100 mg cis-tamoxifen, or the equivalent thereof, to a subject per dosage unit. In still another embodiment, the formulation delivers 100 mg cis-tamoxifen, or the equivalent thereof, to a subject per dosage unit. Thus, for example, a cis-tamoxifen, capsule, a caplet, a tablet, gel or solution, ointment, cream or patch can deliver 0.01 mg, 0.05 mg, 0.1 mg, 0.5 mg, 1 mg, 2 mg, 5 mg, 10 mg, 20 mg, 25 mg, 50 mg, 75 mg, or 100 mg cis-tamoxifen per day. As another non-limiting example, an oral cis-tamoxifen capsule, caplet or tablet can deliver 0.1 mg, 0.2 mg, 0.5 mg, 1 mg, 2 mg, or 5 mg anastrozole per dosage unit.

In one embodiment, the formulation of the present invention delivers 0.1 mg to 500 mg 4-OHT, or the equivalent thereof, to a subject per dosage unit. In another embodiment of the present invention, the formulation delivers from 5 mg to 150 mg 4-OHT, or the equivalent thereof, to a subject per dosage unit. In yet another embodiment, the formulations deliver from 25 mg to 100 mg 4-OHT, or the equivalent thereof, to a subject per dosage unit. In another embodiment of the present invention, the formulation delivers 50 mg to 100 mg 4-OHT, or the equivalent thereof, to a subject per dosage unit. In still another embodiment, the formulation delivers 100 mg 4-OHT, or the equivalent thereof, to a subject per dosage unit. Thus, for example, a 4-OHT capsule, a caplet, a tablet, gel or solution, ointment, cream or patch can deliver 0.01 mg, 0.05 mg, 0.1 mg, 0.5 mg, 1 mg, 2 mg, 5 mg, 10 mg, 20 mg, 25 mg, or 50 mg, or 75 mg, or 100 mg 4-OHT per day. As another non-limiting example, an oral 4-OHT capsule, caplet or tablet can deliver 0.1 mg, 0.2 mg, 0.5 mg, 1 mg, 2 mg, or 5 mg 4-OHT per dosage unit.

In one embodiment, the formulation of the present invention delivers 0.1 mg to 500 mg desmethyltamoxifen, or the equivalent thereof, to a subject per dosage unit. In another embodiment of the present invention, the formulation delivers from 5 mg to 150 mg desmethyltamoxifen, or the equivalent thereof, to a subject per dosage unit. In yet another embodiment, the formulations deliver from 25 mg to 100 mg desmethyltamoxifen, or the equivalent thereof, to a subject per dosage unit. In another embodiment of the present invention, the formulation delivers 50 mg to 100 mg desmethyltamoxifen, or the equivalent thereof, to a subject per dosage unit. In still another embodiment, the formulation delivers 100 mg desmethyltamoxifen, or the equivalent thereof, to a subject per dosage unit. Thus, for example, a desmethyltamoxifen capsule, a caplet, a tablet, gel or solution, ointment, cream or patch can deliver 0.01 mg, 0.05 mg, 0.1 mg, 0.5 mg, 1 mg, 2 mg, 5 mg, 10 mg, 20 mg, 25 mg, or 50 mg, or 75 mg, or 100 mg 4-OHT per day. As another non-limiting example, an oral desmethyltamoxifen capsule, caplet or tablet can deliver 0.1 mg, 0.2 mg, 0.5 mg, 1 mg, 2 mg, or 5 mg desmethyltamoxifen per dosage unit.

In some embodiments, the formulations deliver 0.1 mg to 500 mg endoxifen, or the equivalent thereof, to a subject per dosage unit. In some embodiments of the present invention, the formulation delivers from 5 mg to 150 mg endoxifen, or the equivalent thereof, to a subject per dosage unit. In some preferred embodiments, the formulation delivers 0.01 mg, 0.1 mg, 0.2 mg, 0.5 mg 1 mg, 1.5 mg, 2 mg, 3 mg, 4 mg, 5 mg, 10 mg, 15 mg, 20 mg, 25 mg, 30 mg, 35 mg, 40 mg, 45 mg, or 50 per day. In particularly preferred embodiments, the formulation delivers a dose of 0.2 mg, 0.5 mg, or 1 mg per day. Thus, for example, an endoxifen capsule, a caplet, a tablet, hydroalcoholic gel or solution, ointment, cream or patch can deliver 0.01 mg, 0.1 mg, 0.25 mg, 0.5 mg, 1 mg, 2 mg, 5 mg, 10 mg, 20 mg, 25 mg, 50 mg, 75 mg, or 100 mg endoxifen per day. As another non-limiting example, an oral endoxifen capsule, caplet or tablet can deliver 0.1 mg, 0.2 mg, 0.5 mg, 1 mg, 2 mg, or 5 mg endoxifen per dosage unit.

In some embodiment, the formulations deliver 0.1 mg to 45 mg fulvestrant, or the equivalent thereof, to a subject per dosage unit. In another embodiment, the formulation delivers from 5 mg to 45 mg fulvestrant, or the equivalent thereof, to a subject per dosage unit. In yet another embodiment, the formulations deliver from 10 mg to 40 mg fulvestrant, or the equivalent thereof, to a subject per dosage unit. In another embodiment, the formulations deliver 20 mg to 30 mg fulvestrant, or the equivalent thereof, to a subject per dosage unit. In still another embodiment, the formulations deliver 100 mg fulvestrant, or the equivalent thereof, to a subject per dosage unit. Thus, for example, a fulvestrant capsule, a caplet, a tablet, hydroalcoholic gel or solution, ointment, cream or patch can contain 1 mg, 2 mg, 5 mg, 10 mg, 20 mg, 25 mg, 30 mg, 40 mg, or 45 mg fulvestrant per day. As another non-limiting example, an oral fulvestrant capsule, caplet or tablet can deliver 0.1 mg, 0.2 mg, 0.5 mg, 1 mg, 2 mg, or 5 mg anastrozole per unit dosage unit.

In one embodiment, the formulation delivers 0.1 mg to 250 mg anastrozole, or the equivalent thereof, to a subject per dosage unit. In another embodiment, the formulation delivers from 5 mg to 150 mg anastrozole, or the equivalent thereof, to a subject per dosage unit. In yet another embodiment, the formulations deliver from 25 mg to 100 mg anastrozole, or the equivalent thereof, to a subject per dosage unit. In another embodiment, the formulations deliver 50 mg to 100 mg anastrozole, or the equivalent thereof, to a subject per dosage unit. In still another embodiment, the formulations deliver 100 mg anastrozole, or the equivalent thereof, to a subject per dosage unit. Thus, for example, an anastrozole capsule, a caplet, a tablet, hydroalcoholic gel or solution, ointment, cream or patch can deliver 1 mg, 2 mg, 5 mg, 10 mg, 20 mg, 25 mg, 50 mg, 75 mg, or 100 mg anastrozole per day. As another non-limiting example, an oral anastrozole capsule, caplet or tablet can deliver 0.1 mg, 0.2 mg, 0.5 mg, 1 mg, 2 mg, or 5 mg anastrozole per unit dosage unit.

One of skill in the art will recognize that it is within the scope of the present invention that the compositions described herein may be formulated at higher concentrations of the at least one therapeutic agent, such as 2×, 3×, 4× or 5× the concentrations disclosed herein to allow local delivery of the drug in smaller volumes.

B. Fatty Acid Mixtures

Typically, the fatty acid mixture comprising at least one omega-3 fatty acid ranges from 10% to 90% by weight of a composition disclosed herein. In preferred embodiments, the fatty acid mixture comprising at least one omega-3 fatty acid ranges from 20% to 80%, from 30% to 75%, from 40% to 70%, from 50% to 65%, from 55% to 60% of the compositions. In some preferred embodiments, the compositions disclosed herein are comprised of 30%, 40%, 50%, 60%, 70% or 80% w/w of a fatty acid mixture comprising the at least one omega-3 fatty acid.

In some embodiments, essentially all the fatty acids in the fatty acid mixture is comprised of omega-3 fatty acids, for example, greater than 98%, greater than 99%, greater than 99.5%, and greater than 99.99%. In other embodiments, the fatty acid mixture is comprised of from 50% to 95%, from 60% to 85%, and from 65% to 75% omega-3 fatty acids. In a preferred embodiment, the fatty acid mixture comprises 75% of omega-3 fatty acids. In some other embodiments, the fatty acid mixture comprises from 400 mg/g to 600 mg/g of the at least one omega-3 fatty acid.

In yet other embodiments, the fatty acid mixture comprises a plurality of omega-3 fatty acids. As a non-limiting example, the fatty mixture may comprise a mixture of eicosapentaenoic acid (EPA) and docosahexaenoic acid (DHA). The EPA and DHA in the fatty acid mixture may be present in any suitable ratio. By way of example, without limitations, the fatty acid mixture may comprise EPA and DHA present at an EPA:DHA weight ratio ranging from 1:10 to 10:1. In some embodiments, the EPA:DHA weight ratio ranges from 1:10 to 10:1, from 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1, or from 2:5 to 3:5.

In a preferred embodiment, the EPA:DHA weight ratio of the fatty acid mixture ranges from 2:5 to 3:5. In another embodiment, the EPA:DHA weight ratio of the fatty acid mixture ranges from 1.2 to 2.1. In yet another embodiment, the EPA:DHA weight ratio of the fatty acid mixture ranges from 1.1 to 2.1.

In at least one embodiment, EPA is present at a higher concentration in the fatty acid mixture than DHA. In another embodiment, DHA is present at a higher concentration in the fatty acid mixture than EPA. In yet another embodiment, the fatty acid mixture comprises >95% EPA. In still another embodiment, the fatty acid mixture comprises 20% to 70% EPA. In one embodiment, the fatty acid mixture comprises >95% DHA. In another embodiment, the fatty acid mixture comprises 20% to 70% DHA.

In a further embodiment, the fatty acid mixture comprises 50 mg/g to 80 mg/g EPA triglyceride or phospholipid, and 400 mg/g to 600 mg/g DHA triglyceride or phospholipid. Further, the fatty acid mixture comprises from 850 mg/g to 950 mg/g omega-3 fatty acid.

In another aspect, the fatty acid mixture is a fatty acid oil mixture. The fatty oil mixture according to the present invention may be derived from animal oils, non-animal oils or a combination thereof. In some embodiments, the fatty acid oil mixture is derived from at least one oil selected from a group consisting of a marine oil, a plant-based oil, an algae oil, a microbial oil, and a combination thereof. Marine oils include for example, fish oil, krill oil, and lipid compositions from fish. In a preferred embodiment, the marine oil is fish oil. More preferably, the marine oil is purified fish oil. Plant-based oils include for example, flaxseed oil, canola oil, mustard seed oil, and soybean oil. In some preferred embodiments, the fish oil is anchovy fish oil, tuna fish oil, or cod fish oil.

In at least one embodiment, the fatty acid oil mixture comprises a total amount of omega-3 fatty acid triglycerides or phospholipids of 900 mg/g.

Fatty acid oil mixtures suitable for the present invention are commercially available from variety of sources such as Croda International PLC, Yorkshire, England, Pronova BioPharma Norge AS, Ocean Nutrition, Canada, Lonza, Aker, Martek, Neptune, Mollers, Seven Seas, Vesteralens, Amarin, Omthera Pharmaceuticals, etc. Commercial examples of fatty acid oil mixtures suitable for the present invention comprise the at least one omega-3 fatty acid, includes by way of non-limiting examples: Incromega™ omega-3 marine oil concentrates such as Incromega™ E1070, TG7010 SR, E7010 SR, TG6015, EPA500TG SR, E400200 SR, E4010, DHA700TG SR, DHA700E SR, DHA500TG SR, TG3322 SR, DHA700E SR, DHA500TG SR, TG3322 SR, E3322 SR, TG3322, E3322, and Trio TG/EE (Croda), EPAX6000FA, EPAX5000TG, EPAX4510TG, EPAX2050TG, EPAX7010EE, EPAX5500EE, EPAX5500TG, EPAX5000EE, EPAX5000TG, EPAX6000EE, EPAX6000TG, EPAX6500FA, EPAX6500EE, EPAX6500TG, EPAX4510TG, EPAX2050TG, EPAX7010TG, EPAX7010EE, EPAX6015TG/EE, EPAX4020TG, and EPAX4020EE; MEG-3® EPA/DHA fish oil concentrates (Ocean Nutrition), DHA FNO “functional nutritional oil” and DHA CL “Clear Liquid” (Lonza), Superba™ Krill Oil (Aker), omega-3 products comprising DHA (Martek), Neptune krill oil (Neptune), cod-liver oil products and anti-reflux fish oil concentrate (TG) (Mollers), Lysi Omega-3 fish oil, Seven Seas Triomega® Cod liver oil blend (Seven Seas) etc.

In some embodiments, the compositions deliver to a subject from 50 mg to 800 mg of EPA per gram of fatty acid oil mixture. In some embodiments, the composition delivers 60 mg/g to 500 mg/g of EPA. In other embodiments, the composition delivers from 50 to 150 mg/g of EPA. In a preferred embodiment, the composition delivers to a subject from 300 mg/g to 600 mg/g EPA on a daily basis.

In yet other embodiments, the composition delivers 50 mg/g to 800 mg/g DHA. In some embodiments, the composition delivers 60 mg/g to 500 mg/g of DHA. In other embodiments, the composition delivers from 50 to 150 mg/g of DHA. In a preferred embodiment, the composition delivers to a subject from 650 mg/g to 800 mg/g DHA per day or on a daily basis. In a preferred embodiment, the composition delivers to a subject from 300 mg/g to 400 mg/g DHA on a daily basis. In some embodiments, the composition delivers from 200 mg to 1000 mg/g of omega-3 fatty acids. In a preferred embodiment, the composition delivers from 600 mg/g to 900 mg/g of omega-3 fatty acids. In a more preferred embodiment, the composition delivers from 750 mg/g to 900 mg/g of omega-3 fatty acids.

In yet other embodiments, the composition delivers to a subject on daily administration from 0.5 g to 8 g of fatty acid mixture. In still other embodiments, the compositions deliver to a subject from 0.75 g to 6 g, from 1 g to 4 g, from 1 g to 3 g, or from 1 g to 2.5 g of fatty acid oil mixture administered on a daily basis. In a preferred embodiment, the formulation delivers to a subject from 0.5 g to 1 g of fatty acid oil mixture administered on a daily basis.

C. Vitamin D Compounds

Typically, the at least one vitamin D compound is dissolved, dispersed or suspended in a fatty acid mixture comprising at least one omega-3 fatty acid. In some embodiments, the at least one vitamin D compound may be partly or wholly dissolved in the fatty acid mixture comprising the at least one omega-3 fatty acid. In other embodiments, the at least one vitamin D compound may form a colloid in the fatty acid mixture. In one embodiment, the at least one vitamin D compound is a pre-formed solid dosage form encapsulated in the fatty acid mixture comprising the at least one omega-3 fatty acid. In some embodiments, the at least one vitamin D compound may be dissolved in a pharmaceutically acceptable carrier. In an aspect, the at least one vitamin D compound is present in a pharmaceutically acceptable amount. In some embodiments, the at least one vitamin D compound is selected from a group consisting of cholecalciferol, ergocalciferol, 25-hydroxyvitamin D3, 25-hydroxyvitamin D2, 25-hydroxyvitamin D4, 25-hydroxyvitamin D5, 25-hydroxyvitamin D7, 1-alpha-25-hydroxyvitamin D3, 1-alpha-25-hydroxyvitamin D2, 1-alpha-25-hydroxyvitamin D4, and vitamin D analogs. In one embodiment, the vitamin D compound includes one or more hydroxyl forms, such as a combination of 25-hydroxyvitamin D3 and 25-hydroxyvitamin D2. In a preferred embodiment, the at least one vitamin D compound is cholecalciferol.

The daily dose of the at least one vitamin D compound may range from 10 to 6000 International Units (IU). In some embodiments, the composition delivers to a subject from 100 IU to 800 IU, 400 IU to 6000 IU, from 1000 IU to 4000 IU, or from 2000 IU to 4000 IU of the at least one vitamin D compound on a daily administration. In some preferred embodiments, the composition delivers to a subject a daily dose ranging from 600 IU to 800 IU. In some particularly preferred embodiments, the composition delivers to a subject a daily dose ranging from 10 IU to 400 IU of cholecalciferol.

In some other embodiments, the composition delivers to a subject from 15 μg to 150 μg of the at least one vitamin D compound on a daily administration. In a preferred embodiment, the composition delivers 10 μg, 15 μg, 20 μg, 25 μg or 30 μg of the at least one vitamin D compound on a daily basis. In a particularly preferred embodiment, the composition delivers 15 μg of cholecalciferol per day. In some embodiments particularly suitable for local delivery, the composition delivers to a subject a daily dose ranging from 1 μg to 5 μg of the at least one vitamin D compound. In some preferred embodiments, the composition delivers to a subject a daily dose of 5 μg of cholecalciferol.

D. Excipients

Excipients useful for the present invention include, without limitation, a vehicle, a solvent, a co-solvent, a polymer, a co-polymer, a permeation enhancer, a gelling agent, a polymer, a co-polymer, a stabilization agent, a neutralization agent, a wetting agent, a surfactant, an adhesive, a tackifier, a cross-linking agent, a filler, a film-forming agent, an antifoaming agent, a strengthening agent, a plasticizer, a moisturizer, an emollient, an antioxidant, an antimicrobial, a preservative, or a combination thereof. One of skill will recognize that a composition disclosed herein may comprise one or more of the excipients disclosed herein in any combination appropriate for a desired pharmaceutical formulation or preparation.

i. Vehicles

Vehicles suitable for the purpose of the present invention may be a lipophilic, a hydrophilic or a mixed vehicle. In some preferred embodiments, a composition may comprise a vehicle at 10% to 90% by weight of the composition. In some embodiments, the vehicle is an aqueous vehicle or a non-aqueous vehicle such as an alcoholic vehicle or an oily vehicle or a combination thereof. In at least one preferred embodiment, the vehicle is an alcoholic vehicle capable of dissolving the at least one therapeutic agent. The boiling point of the alcoholic vehicle is preferably below 100° C. at atmospheric pressure to permit rapid evaporation upon contact with skin. Preferred alcoholic vehicles are water miscible alcohols such as lower alcohols, for example, C₁-C₄ alcohols, such as methanol, ethanol, isopropyl alcohol, tert-butyl alcohol, ethylene, glycol, and propylene glycol. Alcoholic vehicle such as fatty alcohols, such as oleyl alcohol and lauryl alcohol are more preferred. The amount of alcoholic vehicle is ranges from 30% to 99.99% by weight of the composition. In some embodiments, the alcoholic vehicle ranges from 35% to 98%, from 40% to 90%, from 40% to 85%, from 45% to 80%, and from 50% to 75% by weight of the composition.

Formulations may also include aqueous vehicles to allow solubilization of hydrophilic molecules, modulate the pH of the formulation. Where used for transdermal delivery, such aqueous vehicles also moisturize the skin. The aqueous vehicles include water, alkalynizing, and buffered solutions, including phosphate buffered solution (PBS) and tris-buffered solution (TBS). In some embodiments, the aqueous vehicle ranges from 0.1% to 60% by weight. In a preferred embodiment, the aqueous vehicle ranges from 15% to 45%, from 20% to 40%, and from 25% to 35%. A preferred aqueous vehicle is PBS.

In some embodiments, the liquid vehicle is an oily vehicle comprising at least one oily component selected from a group comprising animal oils, vegetable oils, a lipophilic compound such as an ester of a long chain fatty acid and the like, or a combination thereof. In some preferred embodiments, the oily vehicle is fish oil. In some embodiments, the aqueous vehicle ranges from 0.1% to 95% by weight. In a preferred embodiment, the oily vehicle ranges from 15% to 45%, from 20% to 40%, and from 25% to 35%. In other embodiments, a mixed vehicle may comprise an alcoholic vehicle and an oily vehicle at ratios ranging from 10:90, 25:75, 40:60, 50:50, 60:40, 75:25, 90:10 (w/w) of alcoholic:oily vehicles. In a preferred embodiment, the vehicle is mixed vehicle, wherein the ratio of alcoholic vehicle to aqueous vehicle is 1:1 (w/w).

ii. Solvents

Suitable solvents in the vehicle include, but are not limited to, organic solvents such as alcohols, acetones, DMSO, polyethylene glycol, fatty acids and fatty alcohols and their derivatives, hydroxyl acids, pyrrolidones, urea, vegetable oils, animal oils such as fish oils, essential oils, and the like or mixtures thereof, and water-miscible solvents such as water miscible alcohols, dimethylsulfoxide, dimethylformamide, water-miscible ether, for example, tetrahydrofuran, water-miscible nitrile, for example acrylonitrile, a water miscible ketone such as acetone or methyl ethyl ketone, an amide such as dimethylacetamide, propylene glycol, glycerin, polyethylene glycol 400, glycofurol, (tetraglycol), and the like, or mixtures thereof. Water-miscible solvents useful for the present invention are glycerin, ethanol, propanol, isopropanol, propylene glycol, polyethylene glycols, or mixtures thereof. Additional solvents that are useful include diglycol monoethyl ether; alkelene glycols, such as propylene glycol; dimethyl isosorbide; and dehydrated alcohol. In a preferred embodiment, the solvent is a dehydrated alcohol, such as absolute alcohol. The concentration of the solvent can also be adjusted. For example, in some embodiments, the formulation includes less than 1% or 1%, 10%, 20%, 30%, 40%, 50%, 60%, 70%, 80% or 90% w/w of solvent. Alternatively, the solvent can be in a range of 1% to 70% w/w. Solvents are typically selected for their ability to dissolve the drug. One of skill in the art will be able to determine the appropriate solvent depending on the API. In a preferred embodiment, the solvent is a dehydrated alcohol, such as absolute alcohol. In a more preferred embodiment, the solvent is a fish oil such as cod fish oil, anchovy oil, and tuna fish oil. In other preferred embodiments, the solvent is ethers. One of skill in the art will appreciate that more than one solvent may be used for preparation the dosage forms described herein.

iii. Co-Solvents

Co-solvents may be added to enhance the solubility of the at least one therapeutic agent. Co-solvents are particularly desirable for topical preparations, wherein the alcoholic vehicles are used for maintaining in solution the at least one therapeutic agent after the alcohol has evaporated after application to the skin. Co-solvents with boiling point ranging from 130° C. to 350° C. are preferred, with those in the range of 150° C. to 200° C. being even more preferred. Co-solvents suitable for use are known in the art and include polyols and polygycols. Co-solvents may be present in the compositions in an amount ranging from 0.01% to 10%, preferably from 1% to 8%, more preferably from 2% to 6% and even more preferably from 3% to 5%, w/w of the pharmaceutical composition.

iv. Plasticizers

Plasticizers may be added to control the softness or pliability of oral dosage forms such as shell of a capsule, caplet or a tablet, or of a transdermal patch layers, and thus, may improve the mechanical properties of the pH-sensitive materials of the coatings on the oral dosage forms and transdermal patch layers. Suitable plasticizers, include, without limitation, petroleum oils (for e.g., a paraffinic process oil, a naphthenic process oil, and an aromatic process oil), squalene, squalane, plant oils, (e.g., olive oil, camellia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof.

In one embodiment, the at least one plasticizer is sorbitol or a glycerol. The amount of plasticizer may vary depending upon the chemical composition of the pharmaceutical preparation. At least one coating and the chemical composition and size of the capsule, and/or the caplet and/or the tablet. In some embodiment, for example, the amount of plasticizer ranges from 10% to 60% by weight of the at least one coating, such as from 10% to 50%, from 20% to 40%, from 30% to 35%. In other embodiments, the ratio of plasticizer to polymer ranges from 10% to 50%, from 20% to 40%, from 30% to 35%.

v. Permeation Enhancers

The compositions may also include a chemical compound to enhance permeation of the active agent through the skin, i.e., a “penetration enhancer” or “permeation enhancer.” Suitable permeation enhancers include those generally useful in conjunction with topical, transdermal and/or transmucosal drug delivery. Examples of suitable permeation enhancers include the following: sulfoxides such as dimethylsulfoxide (DMSO), decylmethylsulfoxide; ethers such as diethylene glycol monoethyl ether and diethylene glycol monomethyl ether; surfactants such as sodium laurate, sodium lauryl sulfate, cetyltrimethylammonium bromide, benzalkonium chloride, poloxomer-231, poloxomer-82, poloxomer-184, Tween-20, Tween-40, Tween-60, Tween-80, and lecithin; fatty acids such as C₆-C₂₀ fatty acids, lauric acid, as isostearic acid, octanoic acid, oleic acid and valeric acid; alcohols such as ethanol and isopropanol, fatty alcohols such as oleyl alcohol and lauryl alcohol; fatty acid esters such as isopropyl myristate, isopropyl palmitate, methylpropionate, butyl stearate, and methyl laurate and ethyl oleate; di(lower)alkyl esters of C6-C22 diacids such as diisopropyl adipate; polyols and esters thereof such as transcutol, (diethylene glycol monoethyl ether), propylene glycol, ethylene glycol, glycerol, butanediol, polyethylene glycol, and polyethylene glycol monolaurate (PEGML; see, e.g., U.S. Pat. No. 4,568,343); alkenes such as long chain alkenes (C7-C16), and amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, menthol, pyrrilidones such as N-methyulpyrrildone and azones such as 1-dodecylazepan-2-one, 2-nonyl-1,3-dioxolane (SEPA 009), sorbitan monolaurate (Span20), and dodecyl-2-dimethylaminopropanoate (DDAIP), terpenes such as Eugenol, cyclodexrtrines, which may be provided at a w/w concentration of from 0.1% to 10%, usually from 2.5% to 7.5%, more usually 5%. Mixtures of two or more enhancers at any suitable ratio may also be used.

vi. Gelling Agents

In another aspect, the gelling agent may be lipophilic or hydrophilic. In some embodiments, the at least one gelling agent is selected from the group consisting of polyacrylic acid, (CARBOPOL®, B.F. Goodrich Specialty Polymers and Chemicals Div. of Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, carboxyvinyl polymers (carbomers), polloxomers, poloxamines, chitosan, dextran, pectins, natural gums, Pemulen® polymeric emulsifiers, and Noveon® polycarbophils, acrylic copolymers such as acrylate/alkylacrylate copolymers, polyacrylamides, cellulose derivatives, ethyl cellulose, hydroxypropyl cellulose, hydroxyethyl cellulose, hydroxypropyl methyl cellulose (HPMC), and carboxymethyl cellulose (CMC), bentones, fatty acid metal salts such as aluminum stearates and hydrophobic silica, or ethylcellulose and polyethylene. Additional thickening agents, enhancers and adjuvants may generally be found in Remington's The Science and Practice of Pharmacy, Meade Publishing Co., United States Pharmacopeia/National Formulary. In some embodiment, the at least one gelling agent is HPMC. In other embodiments, the at least one gelling agent is CMC. In still other embodiments, the at least one gelling agent is Carbopol.

Hydrophilic gelling agents include polyacrylic acid (carbomer), polysaccharides, such as hydroxypropylcellulose, natural gums and clays, and, as lipophilic gelling agents, representative are the modified clays.

The concentration of gelling agent can be adjusted to change the viscosity of the gel. For example, in some embodiments, the formulation includes less than 1%, less than 2% less than 3% less than 4%, less than 5% of the gelling agent. Alternatively, the gelling agent can be in the range of 0.1% to 80% w/w of the composition.

vii. Surfactants

In some embodiments, the surfactant may be a hydrophilic surfactant or a lipophilic surfactant or a combination of both. Suitable examples of surfactants are described above. Commercially available hydrophilic surfactants that may be used are supplied under the tradenames, Cremaphor or Etocas and include, without limitation, Cremaphor EL and RH 40 and Etocas 35 and 40, Cremaphor, RH140 or Etocas 40.

Surfactants particularly suited for transdermal delivery include Tween 85, phospholipids, e.g., plural oleique, TX-100, AOT-tween 80, AOT-DOLPA, AOT-OPE4, CTAB-TRPO, lecithin, and CTAB (cetyltrimethylammonium bromide) and a combination thereof. A surfactant may be present at a concentration of from 5% to 25%, 10% to 20%, 12% to 18%, 14% to 17%, and 15% to 16%.

viii. Neutralizing Agents

In at least one embodiment, a composition may comprise a neutralizing agent. Neutralizing agents useful in the present invention include, without limitation, sodium hydroxide, ammonium hydroxide, potassium hydroxide, arginine, aminomethylpropanol, trolamine, and tromethamine. Those skilled in the art will select a neutralization agent according to the type of gelling agent employed in a formulation. When cellulose derivatives are used as gelling agents, no neutralization agents may be required. In one embodiment, the composition further comprises a neutralization agent, such as, e.g., sodium hydroxide.

ix. Moisturizers

Compositions may optionally comprise at least one moisturizer. Moisturizers are known in the art and be used with alone or in combination with other moisturizers. Moisturizers can emollients and/or humectants. Emollients refer to substances that soften the skin and tend to improve miniaturization of the skin. Emollients are well known in the art and include, without limitation, mineral oil, petroleum, polydecene, isohexadecane, fatty acids, perlargonic, laurc, myristic, plamitic, stearic, isostearic, hydroxystearic, oleic, linoleic, ricinoleic, cocoa butter, safflower oil, olive oil, sunflower oil, cod liver oil, avocado oil, palm oil, sesame oil, soybean oil, silicone oil, polyethylene glycol, squalene, dimethicones, and cyclomethicones and the like. In some embodiments, the composition comprises one or more emollients that are liquid at room temperature. Humectants (i.e., hygroscopic substances that absorb water) from air are suitable for use and include, without limitation, glycerine, propylene glycol, polyolds, sorbitol, maltitol, polydextrose, lactic acid, urea and the like.

x. Tackifiers

Compositions may comprise at least one tackifier. Examples of tackifiers usable in a transdermal preparation, for example for a patch, include, without limitation, resins such as rosin and rosin derivatives (for e.g., a glycerin ester of rosin, hydrogenated rosin, a glycerin ester of hydrogenated rosin, and a pentaerythritol ester of rosin), terpenes and modified terpenes, terpene-phenol resins, aliphatic, cycloaliphatic and aromatic resins (C5 aliphatic resins, C9 aromatic resins, and C5/C9 aliphatic/aromatic resins, aliphatic hydrocarbon resins, aliphatic saturated hydrocarbon resins), hydrogenated hydrocarbon resins, and mixtures thereof.

xi. Adhesives

Adhesive are useful for adhesive formulation of transdermal patches. Examples of such adhesives include, without limitation, pressure sensitive adhesives such as acrylates, natural and synthetics rubbers such as polyisobutylene rubbers, polyisoprene rubbers, polyisoalkylene rubbers, styrene-butadiene-styrene (SBS) block copolymers, styrene-isoprene-styrene (SIS) block copolymers, vinyl acetates, silicone polymers, polybutadienes, acrylic rubbers, vinyl-based high molecular weight materials, such as polyvinyl alkyl ether, polyvinyl acetate, a partially saponified product of polyvinyl acetate, polyvinyl alcohol, and polyvinyl pyrrolidone; cellulose derivatives such as methyl cellulose, carboxymethyl cellulose, and hydroxypropyl cellulose, polysaccharides such as pullulan, dextrin and agar, polysiloxanes, polyurethane elastomers, and polyester elastomers. The adhesives are non-irritating, biocompatible and biodegradable. Selection of an adhesive is such that the patch firmly adheres to the skin of a subject in need of a treatment. Preferably, the concentration of adhesives of the patch is not so high as to injure or irritate the skin of the subject at removal of the patch. In at least some embodiments, a composition may comprise at least one adhesive.

Commercially available adhesives include, without limitation, GELVA 737, GELVA 2655, and GELVA self-crosslinkable acrylic adhesives from Monsanto's Chemical Group, Springfield, Mass., and DUROTEK 87-2516, DUROTAK 87-2194, and DUROTAK 87-2852 self crosslinkable acrylic adhesive available from Starch and Chemical co. Bridgewater, N.J. These crosslinked adhesives find widespread use in the pharmaceutical industry.

xii. Acrylic Adhesives

In some embodiments, composition may comprise an adhesive. In some preferred embodiments, the adhesive may be an acrylic adhesive. Examples of acrylic adhesives include, without limitation, (meth)acrylic acid such as butyl (meth)acrylate, pentyl(methacrylate, hexyl(meth)acrylate, octyl(meth)acrylate, nonyl(meth)acrylate, decyl(meth)acrylate, decyl(meth)acrylate, dodecyl(meth)acrylate, and tridecyl(meth)acrylate, and copolymers thereof, and other monomers copolymerizable therewith.

xiii. Copolymerizable Monomers

In some embodiments, an adhesive may comprise copolymerizable monomers such as (meth)acrylic acid, itaconic acid, crotonic acid, maleic acid, maleic anhydride and fumaric acid; sulfoxyl group-containing monomers such as styrenesufonic acid, sulfopropyl acrylate, (meth)acryloxynaphthalene-sulfonic acid, hydroxyl group-containing monomers such as hydroxyethyl (meth)acrylate, and hydroxypropyl (meth)acrylate; amide group-containing acrylic monomers such as (meth)acrylamide, diemthyl (meth)acrylamide, N-bylacrylamide, tetramethylbuytlacrylamide, and N-methylol(meth) acrylamide; alkylaminoalkyl group-containing acrylic monomers such as aminoethyl methacrylate, dimethylaminoethyl methacrylate, diethylaminoethyl methacrylate, and tertbutyl methacrylate; alkyl esters of acrylic acid containing an ether bond in the molecule thereof such as methoxyethyl emthacrylate, ethoxyethyl (meth)acrylate, butoxyethyl (meth)acrylate, tetrahydrofurfuryl (meth)acrylate, methoxy-ethylene glycol (meth)acrylate, methoxydiethylene glycol (meth)acrylate, methoxypolyethylene glycol (meth)acrylate, and methoxypolypropylene glycol (meth)acrylate; vinyl monomers such as N-(meth)acryoylaminoacid; and functional monomers such as acrylic monomers such as urethane, urea or isocyanate ester of acrylic acid; and vinyl monomers such as (meth)acrylonitrile, vinyl acetate, vinyl propionate, vinyl pyrrolidone, vinyl pyridine, vinyl pyrazine, vinyl piperadine, vinyl piperidone, vinyl pyrimidine, vinyl pyrrole, vinyl imidazole, vinyl caprolactam, vinyl oxazole, vinyl thiazole, vinyl morpholine, styrene, a-methylstyrene and bis(N, N′-dimethylaminoethyl) maleate.

xiv. Crosslinking Agents

Cross-linking may be performed using any suitable cross-linking agent (for example, a chemical cross-linker or irradiation with UV rays or ionizing rays). Cross linking agents can include thermosetting resins such as amino resin, a phenol resin, an epoxy resin, an alkyd resin, and an unsaturated polyester, isocyanate compounds, blocked isocyanate compounds, butyl titanate, polybutyl titanate, aluminum zinc acetate and other multivalent metals, methylol ureas and melamines. Generally, the cross linking agent ranges from 0.005% and 2% of an adhesive formulation.

xv. Fillers

In some embodiments, compositions may comprise at least one filler. Fillers are useful particularly for solid dosage forms and transdermal dosage forms such as a patch. Fillers particularly usable in a patch include, without limitation, calcium carbonate, magnesium carbonate, silicates (for e.g., aluminum silicate and magnesium silicate), silicic acid, barium sulfate, calcium sulfate, calcium zincate, zinc oxide, titanium oxide and a combination thereof.

xvi. Polymers

a. Biodegradable Polymers

In some embodiments, compositions may comprise a biodegradable polymer. Such biodegradable polymer is selected from, without limitation, from a group comprising lactic acid-based polymers such as polylactides, e.g., poly(D,L-lactide), i.e., PLA:glycolic acid-based polymers such as polyglycolides (PGA), for example, Lactel® from Durect, poly(D,L-lactide-co-glycolide), e.g, PLGA (Resomer®); polycaprolactones such as Poly(e-caprolactone) i.e. PCL (Lactel® from Durect), polyanhidrides, poly(sebacic acid) SA; poly(Ricenolic acid)RA; poly(Fumaric acid), FA; poly(Fatty acid dimmer), FAD; poly(tetraphtahlic acid, TA; poly(Isophthalic acid, PA; poly(p-{carboxyphenoxy}methane), CPM; poly(p-{carboxyphenoxy}hetxane), CPH; poly(p-{carboxyphenoxy}propane), CPP; polyamines, polyurethanes, polyesteramides, polyorthoesters, polydioxanones, polyhydroxybutyrates, polyalkyne oxalates, polyamides, polyestaramides, polyacetals, polyketals, polycarbonates, polyorthocarbonates, polysiloxanes, polyphosphazenes, succinates, hyalouronic acids, poly(malic acid); poly(amino acids) such as poly(glutamic) acid, polysiloxanes, polyphosphazenes, succinates, polyalkylene succinate; polyvinylpyrrolidone, polystyrene, synthetic cellulose, polyacrylic acids, polybutyric acid, polyvaric acid, polyethylene glycol, polyhydroxyycellulose, chitin, chitosan, polyorthoesters and copolymers, terpolymers, dimethyl isosorbide, lipids and the like, or mixtures thereof. Biodegradable polymers may be water soluble, acid insoluble or acid soluble. One of skill in the art will be able of select the appropriate polymer based on the formulation and dosage form desired.

b. Acid Soluble Polymers

In some embodiments, compositions may comprise an acid soluble polymer. For example, EUDRAGIT® RL and EUDRAGIT® RS are acrylic resins comprising copolymers of acrylic and methacrylic acid esters. They have a low content of quaternary ammonium groups. The ammonium groups are present as salts and give rise to permeability of the lacquer films. EUDRAGIT® RL and EUDRAGIT® RS, for example, are freely permeable and slightly permeable respectively, independently of pH. The polymers swell in water and in digestive juices, in a pH-independent manner. In the swollen state, they are permeable to water and to dissolved API. Specific examples include EUDRAGIT® RL 30D, EUDRAGIT® RL PO, EUDRAGIT® RL 100, EUDRAGIT® RL 12,5, EUDRAGIT® RS 30D, EUDRAGIT® RS PO EUDRAGIT® RS 100 and EUDRAGIT® RS 12,5. Additional examples include EUDRAGIT® E 100, EUDRAGIT® E 12,5, and EUDRAGIT® E PO. In at least one embodiment, the at least one coating comprises EUDRAGIT® RS 30D. One of skill in the art will recognize that at least some acid soluble polymers listed herein will also be biodegradable.

c. Acid-Insoluble Polymers

In some embodiments, compositions may comprise an acid insoluble polymer. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, N.J. as a powder or a 30% aqueous dispersion; or under the tradename EASTACRYL®, from Eastman Chemical Co., Kingsport, Tenn., as a 30% dispersion). They are insoluble in acids and pure water. They become soluble in neutral to weakly alkaline conditions. Their permeability is pH-dependent, and above pH 5.0, they become increasingly permeable. Specific examples include EUDRAGIT® L100-55, EUDRAGIT® L30D-55, EUDRAGIT® L100, EUDRAGIT® L100 12,5, EUDRAGIT® S100, EUDRAGIT® S12,5, and EUDRAGIT® FS 30D. Additional examples include EUDRAGIT® E100, EUDRAGIT® E 12,5, and EUDRAGIT® PO. In at least one embodiment, the preparation comprises EUDRAGIT® L100-55. In some preferred embodiments, the preparation comprises EUDRAGIT® L30D 55. One of skill in the art will recognize that at least some acid insoluble polymers listed herein will also be biodegradable.

d. Water-Soluble Polymers

In some embodiments, compositions comprise a water soluble polymer. Water soluble polymers that are suitable for use may be synthetic or natural. Synthetic water soluble polymers suitable for use include, without limitation, Poly(ethylene) gluycol (PEG), polyvinyl pyrrolidone (PVP), polyvinylpyrrolidone-vinyl acetate (PVP-VA), polyvinyl alcohol (PVA), polyacrylic acid (PAA), polyacrylamides, N-(2-Hydroxypropyl) methylacrylamide (HPMA), divinyl ether-maleic anhydride (DIVEMA), polyoxazoline-polyphosphates, polyphosphazenes. Natural water soluble polymers include xanthum gum, pectins, chitosan derivatives, dextrans, carageenans, guar gum, cellulose ethers like HPMC, HPC, HEC, Na-CMC, hyalouronic acid, albumin, starch and starch-based derivatives. In some embodiments, PVP is preferred. One of skill in the art will recognize that at least some water soluble polymers listed herein will also be biodegradable.

xvii. Film-Forming Agents

polysaccharides, polysaccharide derivatives, polymethacrylates, cellulose-based polymers, ethylcellulose, hydroxypropyl methyl cellulose (HPMC), cellulose acetate phthalate, cellulose acetate trimellitate (cellulose acetate succinate, hydroxypropylmethylcellulose phthalate, hydroxypropyl-methylcellulose succinate), polyvinylpyrrolidone (PVP), polyvinyl derivatives (polyvinyl acetate phthalate), half esters of the copolymeristate of styrene and maleic acid, and copolymeristate of vinyl acetate and crotonic acid, and combinations and thereof. In one embodiment, the film-forming agent comprises HPMC.

xviii. Wetting Agents

In some embodiments, compositions may comprise a wetting agent. Wetting agents enhance dissolution and disintegration of the capsules or tablets. Examples of wetting agent include, without limitation, ethoxylated aliphatic alcohols, polyoxyethylene surfactants, carboxylic esters, polyethylene glycol esters, anhydrosorbitol esters, ethoxylated derivatives of anhydrosorbitol esters, glycol esters of fatty acids, carboxylic amides, monoalkylamine condensates and polyoxyethylene fatty acid amides. Commercially available wetting agents that can be used as wetting agents include without limitation, Solutol, polysorbate 20, polysorbate 60, polysorbate 80, Brij 35, 58, 78, 98, Myrj 52, 59, Crodesta SL40, SPAN 60, poloxomers, poloxamines etc. Accordingly in at least one embodiment, a composition may comprise a wetting agent selected from a group comprising Solutol, polysorbate 20, polysorbate 60, polysorbate 80, Brij 35, 58, 78, 98, Myrj 52, 59, Crodesta SL40, SPAN 60, poloxomers, poloxamines. Solutol or polysorbate 80 are preferred.

xix. Anti-Foaming Agents

Antifoaming agent are useful in the present invention to prevent bloating and include, without limitation, antifoaming agents that are oil-based, powder-based, silicon based, EO/PO based, sorbitan sequoleate, or alkyl polyacrylates. Examples of silicone based antifoaming agent includes simethicone. When the antifoaming agent is used, it may be used as an oil-based emulsion. Accordingly, in some embodiments, compositions may comprise an antifoaming agent. A preferred antifoaming agent is a silicon emulsion, a sorbitan sequoleate, or an alkyl polyacrylate.

xx. Strengthening Agents

The strengthening agent or a stabilizing agent is selected from the group consisting of polydextrose, cellulose, or it is derivatives, poly-L-ornithine, maltodextrin, guar gum, gelatin, alginates, and gum Arabic. The strengthening agent may be present from 0.1% to 20% by weight of the weight of the preparation. In a preferred embodiment, the strengthening agent is polydextrose. In other preferred embodiments, the strengthening agent is poly-L-ornithine.

xxi. Antioxidants

In another aspect, compositions described herein comprise an antioxidant. In some embodiments, the antioxidant is selected from the group consisting of alpha-tocopherol (vitamin E), butylhydroxyanisoles (BHA), butylhydroxytoluene (BHT), ascorbic acid and pharmaceutically acceptable salts and esters thereof, propyl gallate, citric acid and pharmaceutically acceptable salts thereof, malic acid and pharmaceutically acceptable slats thereof, and sulfite salts and mixtures thereof.

xxii. Anti-Microbial Agents

The compositions of the present invention may also comprise at least one antimicrobial agent. The infections that may be treated by the methods and compositions of the present invention may be any opportunistic infection of a wound by a bacterium, or a multiple infection of more than one species of bacteria. The action of the antimicrobial agent can be either bacteriostatic wherein the antibiotic arrests the proliferation of, but does not necessarily kill, the microorganism or the activity of the antibiotic can be bacteriocidal and kill the organism or a combination of activities. Antibiotics suitable for use in the wound management methods of the present invention include, but are not limited to, .beta.-lactams (penicillins and cephalosporins), vancomycins, bacitracins, macrolides (erythromycins), lincosamides (clindomycin), chloramphenicols, tetracyclines, aminoglycosides (gentamicins), amphotericns, cefazolins, clindamycins, mupirocins, sulfonamides and trimethoprim, rifampicins, metronidazoles, quinolones, novobiocins, polymixins, tetracyclines, and Gramicidins and the like and any salts or variants thereof. Antiseptic agents may also be used and include ethyl para-hydroxybenzoate, propyl para-hydroxybenzoate, and butyl para-hydroxybenzoate.

xxiii. Controlled Release Agents

Pharmaceutical preparations disclosed herein may comprise a controlled release agent. Examples of controlled release agent suitable for use include, without limitation, waxes, including synthetic waxes, microcrystalline waxes, paraffin wax, carnauba wax, and beeswax; polyethoxylated castor oil derivatives, hydrogenated oils, glyceryl mono-, di-tribenates, long chain alcohols, such as stearyl alcohol, cetyl alcohol, and polyethylene glycol; and mixtures thereof. Non-digestible waxy substances such as hard paraffin wax are preferred. The controlled release agents can be present in an amounts ranging from 1% to 50% by weight of formulation. In some preferred embodiments, the controlled release agent is present at amounts of at least 5%, at least 10%, at least 15%, or at least 20%, at least 25% by weight of formulation. In some other embodiments, the controlled release agents are present in amounts greater than 5%, greater than 10%, greater than 15%, greater than 20%, greater than 25% or 50% or less by weight of the formulation.

For delayed-release pharmaceutical preparations of oral dosage forms, acid-insoluble polymers are preferred, (e.g., as coating material forenteric coating of capsules, caplets, and tablets).

xxiv. Other Optional Additives

Compositions disclosed herein may comprise other optional additives such as colorants, flavorants, opacifiers, sweeteners, and the like.

The formulations disclosed herein are designed to provide maximum delivery and uptake in the affected tissues with rate of release as needed (for example, rapid or sustained release) throughout the region to be treated with little to no increase in the systemic blood levels of the therapeutic agents. Such formulations preferably avoid hepatic metabolism. The present invention specifically provides that the pharmaceutical preparations are suitable for oral, a transdermal, and a parenteral administration. Pharmaceutical preparations that are useful with composition of the present invention include but are not limited to tablets, caplets, capsules, pills, dragees, suppositories, solutions, suspensions, emulsions, ointments, creams, lotions, and gels. For oral mode of administration, preferred forms include tablets, caplets, capsules, lozenges, powders, syrups, and the like.

The present invention specifically provides that the pharmaceutical preparations are suitable for local delivery. In some embodiments, local delivery of the compositions disclosed herein may be transdermal, transpapillary or intraductal delivery to a tissue (such as breast tissue and ducts). For topical application and suppositories, preferred formulations comprise gels, solutions, oils, ointments, lotions, creams, and emulsions. The term topical or transdermal application denotes any mode of delivery of a composition from the surface of a subject's skin, through stratum corneum, epidermis, and dermis layers, and into microcirculation. This is typically achieved by diffusion down a concentration gradient. The diffusion may occur via intracellular penetration, intercellular penetration, transappendageal penetration for example, through the hair follicles, sweat and sebaceous glands, or any combination thereof. The preparations may be applied to any location on a breast. In a preferred embodiment, a preparation is applied directly over the spot of suspected disorder, hyperplasia, or cancer. In a more preferred embodiment, the composition may be applied directly on the nipple or the mammary papilla. In at least one embodiment, a preferred formulation for intraductal delivery is a gel or a solution.

E. Gels and Solutions

In some embodiments, the pharmaceutical preparation comprising a composition disclosed herein is formulated in a gel or a solution. The gels and solutions disclosed herein are particularly suited for local delivery to a tissue. In some preferred embodiments, the preparation is a hydroalcoholic gel or a hydroalcoholic solution. In other preferred embodiments, the preparation is a hydrogel. In one aspect, the gel or solution comprises at least one therapeutic agent, a fatty acid mixture comprising at least one omega-3 fatty acid, and at least one vitamin D compound. In at least one embodiment, the gel or solution further comprises at least one excipient. Such excipients or components have been described above and are suitable for use in the preparations disclosed herein.

When preparation is formulated for infusion into a duct, such as breast duct, by using a biodegradable material, it may be demonstrated that the cycle of repeated infusion and degration of the biomaterial within a duct will allow for a greater volume of material to be infusion into the duct over time. They typically comprise gelling agents, natural and/or synthetic polymers and copolymers, that are crosslinked or derivatized for crosslinking, for example, with mercaptosuccinic acid (also known as thiomalic acid) or dimercaptosuccinic acid. Increasing the extent of cross-linking the gel matrix results in a gel with smaller pores. In some embodiments, a nanocarrier can be incorporated into the gel by incorporating a previously produced nanocarrier into the gel reaction system at an appropriate point, such as to the polymer scaffold solution or crosslinker solution, prior to mixing of the scaffold with the cross-linker solution, or after mixing of the scaffold with the crosslinker but before an undue amount of cross-linking has occurred. The API can be either physically entrapped or modified drugs with cleavable bonds can be physically incorporated or covalently linked into the gel to provide controlled release, which is otherwise not possible for highly hydrophilic drugs which traverse easily through the gel.

Polymers useful for the present invention have been described above. In some preferred embodiments, the polymers are starches, polyethylene glycols, or PVA. In at least one embodiment, the at least one gelling agent is HPMC, CMC or polyacrylic acid. For example, in some embodiments, the preparation includes less than 1%, less than 2% less than 3% less than 4%, less than 5% of the gelling agent. In other embodiments, the gelling agent can be in the range of 0.1% to 80% w/w. In some embodiments, the preparation comprises no gelling agent.

In one aspect, the preparations disclosed herein have low viscosity at room temperature (RT) i.e., between 20° C. and 25° C. In another aspect, the preparations have a higher viscosity at room temperature (RT) i.e., between 20° C. and 25° C. In some embodiments, the viscosity of the preparation is suitable for transdermal or transpapillary penetration. In other embodiments, the viscosity is suitable for intraductal delivery. In some embodiments, the property of preferred gel preparations are that they are low viscosity on infusion, but following a period of time, become more viscous, and then a solid material or a hydrogel. Hydrogels are three dimensional, hydrophilic, polymeric networks which swell in water without dissolving and retain large quantities of water. The concentration of gelling agent can be adjusted to change the viscosity of the preparation. In some embodiments, the preparation has a viscosity of between 5000 and 0.5 cp at RT. In other embodiments, the preparation has a viscosity from 2500 cp to 0.5 cp, from 1000 cp to 0.5 cp, from 750 cp to 0.5 cp, from 500 cp to 0.5 cp, from 250 cp to 0.5 cp, from 100 cp to 0.5 cp, or from 50 cp to 0.5 cp at RT. In some embodiments, the preparations have a viscosity less than 10 cp, less than 5 cp, or less than 1 cp at 25° C. at RT.

In some embodiments, the gel or solution comprises a vehicle, a solvent, a co-solvent, a permeation enhancer, a neutralization agent, a surfactant, an antioxidant, an antimicrobial, a preservative, or a combination thereof. In some preferred embodiments, the gel comprises a vehicle, a permeation enhancer, a gelling agent, and water.

In some embodiments, the vehicle is a non-aqueous vehicle (such as an alcoholic vehicle or an oily vehicle), an aqueous vehicle, or a combination thereof. The amount of an alcoholic vehicle ranges from 30% to 99.99% by weight of the preparation. In some embodiments, the alcoholic vehicle ranges from 35% to 98%, from 40% to 90%, from 40% to 85%, from 45% to 80%, and from 50% to 75% by weight. In some embodiments, the aqueous vehicle ranges from 0.1% to 60% by weight. In a preferred embodiment, the aqueous vehicle ranges from 15% to 45%, from 20% to 40%, and from 25% to 35%. In some preferred embodiments, aqueous vehicle is water or PBS. In other preferred embodiments, preferred alcoholic vehicle is ethanol, isopropyl alcohol, propanol or a combination thereof. In at least one preferred embodiment, the vehicle is a mixed vehicle comprising an alcoholic vehicle and an aqueous vehicle, wherein the ratio of alcoholic vehicle to aqueous vehicle is 1:1 (50%:50%) (w/w). In a more preferred embodiment, the vehicle is an oily vehicle. A preferred oily vehicle suitable for use in the invention is fish oil. In a still more preferred vehicle, the vehicle is mixed comprising an oily vehicle and an aqueous vehicle such as an oil-in-water or a water-in-oil vehicle. In another preferred embodiment, the vehicle comprises greater than 75% non-aqueous vehicle by weight of the composition. In other embodiments, the mixed vehicle comprises an alcoholic vehicle and an oily vehicle.

The pH of the pharmaceutical preparation ranges from pH 4 to pH 11, from pH 5 to pH 10, from pH 6 to pH 9, and from pH 7 to pH 8.

In at least one embodiment, the solvent is a dehydrated alcohol, such as absolute alcohol. The concentration of the solvent can also be adjusted to change the viscosity of the preparation. For example, in some embodiments, the preparation includes less than 1% or 1%, 10%, 20%, 30%, 40%, 50%, 60%, 70%, or 80% w/w of solvent. In some preferred embodiments, the solvent can be in a range of 1% to 70% w/w.

Permeation enhancers suitable for the present invention have been described above. In some embodiments, permeation enhancers may be provided at a w/w concentration from 0.1% to 10%, usually from 2.5% to 7.5%, more usually 5%. In some preferred embodiments, the permeation enhancer is an ether, a sulfoxide, a poloxomer, a pyrrolidone, an azone or a fatty alcohol. In at least one embodiment, the permeation enhancer is oleyl alcohol or lauryl alcohol. In one embodiment, the gel further comprises a neutralization agent. Sodium hydroxide is a preferred neutralizing agent. Depending on the nature of the selected ingredient, it may be advantageous to include a surfactant. In some preferred embodiments, surfactant is SDS, cetrimide, Capmul, Cremaphor, or Tween 85. In some embodiments, preparations may comprise a surfactant ranging from 0.01% to 6% of the total preparation. In more preferred embodiments, the surfactant is 0.01%, 0.02%, 0.03%, 0.04%, 0.05%, 0.1%, 0.5% of the total preparation.

In some preferred embodiments, the moisturizer ranges from 0.01% to 30% of the total preparation. In more preferred embodiments, the composition comprises 0.01%, 0.05%, 0.1%, 0.5%, 1% of the total preparation.

Additionally, the present invention may optionally include salts, emollients, humectants, stabilizing agents, antimicrobials, and fragrances.

In some preferred embodiments, the gel comprises: (a) API (i) at least one therapeutic agent selected from a group consisting of SERMs, SERDs, AI, or a combination thereof, (ii) a fatty acid mixture comprising at least one omega-3 fatty acid, and (iii) at least one vitamin D compound; and (b) fish oil.

In other preferred embodiments, the gel comprises: (a) API (i) at least one therapeutic agent selected from a group consisting of SERMs, SERDs, AI, or a combination thereof, (ii) a fatty acid mixture comprising at least one omega-3 fatty acid, and (iii) at least one vitamin D compound, (b) fish oil, and (c) a gelling agent.

In another preferred embodiment, the gel comprises 0.0 g-15 g of at least one therapeutic agent such as a SERM, a SERD, an AI or a combination thereof, 1 g to 10 g of fatty acid mixture comprising at least one omega-3 fatty acid, 100-6000 IU at least one vitamin D compound, 0.01 g 50 g gelling agent, and fish oil (qs) to 100 g.

In another preferred embodiment, the gel comprises: 0.0 g to 15 g of at least one therapeutic agent such as a SERM, a SERD, an AI or a combination thereof, 1 g to 10 g of fatty acid mixture comprising at least one omega-3 fatty acid, 100 IU to 6000 IU at least one vitamin D compound, 0.5 g to 50 g permeation enhancer, 30 g to 98 g fatty alcohols, 0.01 g to 50 g gelling agent, and fish oil (qs) to 100 g.

An exemplary embodiment is shown in the table 1 below.

| | Composition (100 g) |
|---|---|
| Desmethyltamoxifen | 12% |
| Fatty acid mixture comprising EPA triglyceride | 4 g |
| Cholecalciferol | 3000 IU |
| Permeation enhancer (lauryl alcohol) | 10% |
| Gelling agent (Carbopol) | 5% |
| Ethanol/Isopropanol (1:1) | 2% |
| Cod fish oil | q.s. to 100% |

In one aspect, the present invention relates to a pharmaceutical composition for topical and/or intraductal administration to a subject, wherein the composition comprises: (i) 0.01% to 15% of a SERM, a SERD, an AI or a combination thereof, or pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; and (iv) 10% to 90% vehicle.

In one aspect, the present invention relates to a pharmaceutical composition for topical and/or intraductal administration to a subject, wherein the composition comprises: (i) 0.01% to 15% of a SERM, a SERD, an AI or a combination thereof, or pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; and (v) q.s. 100% (w/w) water.

In one aspect, the present invention relates to a pharmaceutical composition for topical and/or intraductal administration to a subject, wherein the composition comprises: (i) 0.01% to 15% of a SERM, a SERD, an AI or a combination thereof, or pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; and (v) 0.1% to 80% at least one gelling agent.

In one aspect, the present invention relates to a pharmaceutical composition for topical and/or intraductal administration to a subject, wherein the composition comprises: (i) 0.01% to 15% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; and (v) 0.1% to 80% at least one gelling agent.

In one aspect, the present invention relates to a pharmaceutical composition for topical and/or intraductal administration to a subject, wherein the composition comprises: (i) 0.01% to 15% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; and (vi) q.s. 100% (w/w) water.

In one aspect, the present invention relates to a pharmaceutical composition for topical and/or intraductal administration to a subject, wherein the composition comprises: (i) 0.01% to 15% of a SERD selected from a group consisting of fulvestrant, a ARN-810, and a CH4986399, and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; and (vi) q.s. 100% (w/w) water.

In one aspect, the present invention relates to a pharmaceutical composition for topical and/or intraductal administration to a subject, wherein the composition comprises: (i) 0.01% to 15% of an AI selected from a group consisting of anastozole, exemestrane, and letrozole, and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; and (vi) q.s. 100% (w/w) water.

In one aspect, the present invention relates to a pharmaceutical composition for topical and/or intraductal administration to a subject, wherein the composition comprises: (i) 10% to 15% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; and (vi) q.s. 100% (w/w) water.

In one aspect, the present invention relates to a pharmaceutical composition for topical and/or intraductal administration to a subject, wherein the composition comprises: (i) 0.01% to 15% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; (vi) 0.1% to 80% at least one gelling agent; and (vii) q.s. 100% (w/w) water.

In one aspect, the present invention relates to a pharmaceutical composition for topical and/or intraductal administration to a subject, wherein the composition comprises: (i) 0.01% to 15% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising EPA triglyceride; (iii) 10 IU to 6000 IU of cholecalciferol or pharmaceutically acceptable salts thereof; (iv) 10% to 90% ethanol; (v) 0.1% to 10% permeation enhancer; (vi) 0.1% to 80% at least one gelling agent; and (vii) q.s. 100% (w/w) water.

In one aspect, the present invention relates to a pharmaceutical composition for topical and/or intraductal administration to a subject, wherein the composition comprises: (i) 0.01% 5% of a SERM selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof; (ii) 10% to 90% of a fatty acid mixture comprising EPA:DHA in a form selected from a triglyceride or a phospholipid; (iii) 10 IU to 4000 IU of cholecalciferol or pharmaceutically acceptable salts thereof; (iv) 10% to 90% ethanol; (v) 0.1% to 10% permeation enhancer; (vi) 0.1% to 10% at least one gelling agent such as HPMC, CMC, Carbopol, and/or polyacrylic acid; and (vii) q.s. 100% (w/w) PBS.

In one aspect, the present invention relates to a pharmaceutical composition for topical and/or intraductal administration to a subject, wherein the composition comprises: (i) 0.01% to 15% of a SERM, a SERD, an AI or a combination thereof, and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; (vi) 0 to 10% (w/w) of a moisturizer, such as glycerine, and (vii) q.s. 100% (w/w) water.

In one aspect, the present invention relates to a pharmaceutical composition for topical and/or intraductal administration to a subject, wherein the composition comprises: (i) 0.01% to 15% of a SERM, a SERD, an AI or a combination thereof, and pharmaceutically acceptable salts thereof; (ii) 10% to 90% of a fatty acid mixture comprising at least one omega-3 fatty acid; (iii) 10 IU to 6000 IU of at least one vitamin D compound or pharmaceutically acceptable salts thereof; (iv) 10% to 90% vehicle; (v) 0.1% to 10% permeation enhancer; (vi) 0.1% to 5% (w/w) of an anti-oxidant, such as alpha-tocopherol; and (vii) q.s. 100% (w/w) PBS.

In one embodiment, the composition comprises: (i) 0.01% to 5% of a SERD, a SERM, an AI, or a combination thereof, and pharmaceutically acceptable salts thereof; (ii) 60% of a fatty acid mixture comprising greater than 99% EPA triglyceride; (iii) 800 IU of cholecalciferol and pharmaceutically acceptable salts thereof; (iv) 30% ethanol; (v) 2% permeation enhancer; (vi) 5% polyacrylic acid; and (vii) q.s. 100% (w/w) fish oil.

In one embodiment, the composition comprises: (i) 0.01% to 10% of a SERD, a SERM, an AI, or a combination thereof, and pharmaceutically acceptable salts thereof; (ii) 60% of a fatty acid mixture comprising greater than 99% EPA triglyceride or phospholipid; (iii) 800 IU of cholecalciferol and pharmaceutically acceptable salts thereof; (iv) 30% oleyl alcohol; (v) 5% polyacrylic acid; and (vi) q.s. 100% (w/w) fish oil.

In another embodiment, the composition comprises: (i) 0.01% to 15% at least one therapeutic agent; (ii) 20% to 60% of a fatty acid mixture comprising at least one omega-3 fatty acid triglyceride selected from a group consisting of EPA, DHA, ALA, HTA, SDA, ETE, ETA, EPA, HPA, DPA, clupanodonic acid, tetracosapentaenoic acid, tetracosahexaenoic acid, and a combination thereof; (iii) 10 IU to 6000 IU of at least one vitamin D compound; and (iv) a vehicle; wherein the vehicle comprises w/v of a gel or a solution (a) 60% to 80% of non-aqueous vehicle; (b) a sufficient amount of fish oil, and (c) 0.5% to 6% by weight of a gelling agent.

In another embodiment, the composition comprises: (i) 0.01% to 15% of a SERM, a SERD, an AI or a combination thereof; (ii) 20% to 60% of a fatty acid mixture comprising at least one omega-3 fatty acid triglyceride selected from a group consisting of EPA, DHA, ALA, HTA, SDA, ETE, ETA, EPA, HPA, DPA, clupanodonic acid, tetracosapentaenoic acid, tetracosahexaenoic acid, and a combination thereof; (iii) 10 IU to 6000 IU of at least one vitamin D compound; and (iv) a vehicle; wherein the vehicle comprises w/v of a gel or a solution (a) 50% of fatty alcohol; (b) 0.5% to 6% by weight of a gelling agent; and (c) a sufficient amount of fish oil.

In another embodiment, the composition comprises: (i) 0.01% to 15% of at least one therapeutic agent selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652, ERA-923, fulvestrant, ARN-810, CH4986399, anastozole, exemestrane, and letrozole, and pharmaceutically acceptable salts thereof, or a combination thereof; (ii) 20% to 60% of a fatty acid mixture comprising at least one omega-3 fatty acid triglyceride or phospholipid selected from a group consisting of EPA, DHA, ALA, HTA, SDA, ETE, ETA, EPA, HPA, DPA, clupanodonic acid, tetracosapentaenoic acid, tetracosahexaenoic acid, and a combination thereof; (iii) 10 IU to 6000 IU of at least one vitamin D compound; and (iv) a vehicle; wherein the vehicle comprises w/v of a gel or a solution (a) 10% to 80% of alcoholic vehicle or aqueous vehicle; (b) a sufficient amount of fish oil, and (c) 0.5% to 6% by weight of a gelling agent.

In another embodiment, the composition comprises: (i) 0.01% to 15% of at least one therapeutic agent selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652, ERA-923, fulvestrant, ARN-810, CH4986399, anastozole, exemestrane, and letrozole, and pharmaceutically acceptable salts thereof, or a combination thereof; (ii) 100% to 90% of a fatty acid mixture comprising at least one omega-3 fatty acid triglyceride or phospholipid selected from a group consisting of EPA, DHA, ALA, HTA, SDA, ETE, ETA, EPA, HPA, DPA, clupanodonic acid, tetracosapentaenoic acid, tetracosahexaenoic acid, and a combination thereof; (iii) 10 IU to 6000 IU of at least one vitamin D compound; and (iv) a sufficient amount of fish oil.

The many factors and methods relating to topical drug delivery are reviewed in Remington: The Science and Practice of Pharmacy, Alfoso R. Genaro (Lippincott Williams & Wilkins, 2000), at pages 836-58, and in Percutaneous absorption: Drugs, Cosmetics Mechanisms Methodology, Broaugh and Maiback (Marcel Dekker, 1999). As these publications evidence, those of skill in the art can manipulate the various factors and methods to achieve efficacious percutaneous or topical delivery.

F. Devices

Preparations comprising compositions disclosed herein may be administered by any means suitable to apply the composition to the skin of a subject, for example, without limitation, manually with or without an applicator, as a patch, as a spray aerosolized or non-aerosolized, from a pressurized container or non-pressurized container. In some embodiments, the compositions are administered in metered doses, such as from a metered dose applicator or from an applicator comprising a single dose of the composition.

In yet another aspect, the preparations may be administered to a subject in combination with other delivery technologies including transdermal and intraductal technologies, including, without limitation, iontophoretic and electroporation methods (applying micro-electric potential to the skin), the application of, non-cavitational and cavitational ultrasound to drive potentiators of the active pharmaceutical ingredients (API) into the skin, application of magnetic field such as a permeation enhancer, thermal ablation, microneedles, microdermabrasion, and mechanical devices to give positive pressure, and also the use of a nano-fabricated patch with different gradients of drug loading.

Accordingly, the present invention in one aspect provides a device for administering the compositions. Some of the therapeutic agents of the present invention, such as some of the SERMs, may be susceptible to adverse effects of light and thus, in some embodiments, the device may be opaque.

In some embodiments, the device comprises a drug reservoir containing a composition. The reservoir may be of any shape, size, configuration and any material suitable for containing a composition. For example, the reservoir may be flexible or rigid, may be of a unitary construction or maybe formed from multiple pieces secured together, such as by laminating, heat sealing, welding, riveting, etc. The reservoir may comprise of a rolled wall, two walls substantially parallel joined at the vicinity of their periphery (where the walls may be flexible or deformed or deformable, formed by a thermoformed blister, or rigid), or a bottom wall and a cylindrical wall, or any other configuration suitable for containing the composition. In some embodiments, the reservoir comprises a bag, a pouch, a sachet, a blister, an ampoule, a pipette, a vial, a canister, a tubing, a catheter, or a bottle. In some embodiments, the reservoir comprises a deformable wall that is adapted to actuate flow of composition when deformed. In some embodiments, a reservoir may be a sponge-like section in the device.

In some embodiments, the device comprises a single reservoir. In other embodiments, the device comprises two or more reservoirs. Each reservoir may contain a single dose of a composition or may contain any amount of composition that is suitable for the purpose of the present invention.

In some embodiments, the reservoir comprises a gel or a matrix, in which a composition is stored. In some preferred embodiments, a reservoir comprises a polyacrylamide gel, a poloxomer gel, or polymer matrix of polyvinylpyrrolidone (PVP) and ethylcellulose. In other embodiments, the gel is a hydroalcoholic gel. Suitable carriers for the compositions include any aqueous medium, oil, emulsion, ointment and a combination thereof that will allow the compositions to be delivered to the target tissue. In some embodiments, compositions may be encapsulated in a reverse micelle formed and/or stabilized with a nonionic surfactant in the matrix. Surfactants particularly suited for transdermal delivery include Tween 85, phospholipids, e.g. plural oleique, TX-100, AOT-tween 80, AOT-DOLPA, AOT-OPE4, CTAB-TRPO, lecithin, and CTAB (cetyltrimethylammonium bromide) and a combination thereof. A surfactant may be present at a concentration of from 5% to 25%, 10% to 20%, 12% to 18%, 14% to 17%, 15%, 16%, and 17% by weight.

G. Transdermal Devices

In one aspect, the transdermal device, such as a patch, a tape, a sheet, a dressing, or any other form known to one of skill in the art comprises a composition described herein, and at least one adhesive layer comprising an adhesive formulation. Compositions may be incorporated directly into the adhesive layer of the device. Accordingly, in some embodiments, the device may be a single-layer drug-in-adhesive or a multi-layer drug-in adhesive.

In other embodiments, the device comprises a backing layer. Preferably, the backing layer is impermeable. In some embodiments, the backing layer is flexible such that the device conforms to the skin. In some embodiments, the device comprises a composition, and a backing layer, and at least one adhesive layer. In some preferred embodiments, the drug reservoir is enclosed on one side with an impermeable backing layer and an adhesive layer comprising an adhesive formulation that contacts the skin on the other side. Preferably, the device delivers compositions in amounts sufficient for at least three days.

The backing layer can be any material suitable to support the adhesive layer. It can be an expandable layer or a non-expandable layer. The backing layer may be a cloth, non-woven fabric, polyurethane, polyesters such as polyethylene terephthalate, ethylene-vinyl acetate, polyvinyl acetate, polyvinylidene chloride, polyethylene, for example, low density polyethylene and high density polyethylene, polyethylene terephthalate, an aluminum sheet, rayon, and a combination or a composite thereof. Backing layers that are polyethylene terephthalate-aluminum-polyethylene composite are also suitable.

In some embodiments, a device may further comprise a release liner, which may optionally be rate-limiting. In some embodiments, a composition may be applied or coated onto the release liner. Non-limiting examples of release liner include a vinyl chloride film, a polyethylene film, a polypropylene film, a polyester film, polyethylene-coated paper coated with a suitable fluoropolymer or silicone based paper, a polyethylene terephthalate separator according to a pharmaceutical additive specification, and a release paper (exfoliate paper). A pharmaceutical composition as disclosed herein can be dissolved, dispersed or suspended into a suitable vehicle as described above and coated on to the release liner. The release liner is then dried and laminated onto a backing using conventional methods.

In other embodiments, the transdermal drug delivery device such as a patch may comprise at least two distinct layers in addition to the backing layer. A first layer comprising a composition described herein is attached to the backing layer and serves as a drug reservoir (a reservoir layer). The second layer, (i.e., a rate controlling layer), comprises a pressure sensitive adhesive layer that is attached a surface of the first layer opposed to a surface in contact with the backing. The rate-controlling layer serves to attach to a subject's skin (a skin contacting layer) and controls the rate of delivery of the drug to a subject.

In yet other embodiments, the transdermal drug delivery device comprises at least three distinct layers in addition to a backing layer. A first layer comprising a composition disclosed herein is attached to the backing layer and serves as a drug reservoir (a reservoir layer). A second layer (i.e., a rate controlling layer) comprises a rate-controlling membrane that is attached to a surface of the first layer opposed to a surface in contact with the backing layer. A third layer comprises a pressure sensitive adhesive that is attached to a surface of the membrane that is opposed to the surface of the membrane in contact with the first layer. The third layer contacts a skin of a subject when the device is used. This third layer is referred to as the “skin-contacting layer.”

Adhesive layer typically comprises an adhesive formulation. Pressure sensitive adhesives in the adhesive formulation may be any adhesive that is useful for transdermal drug delivery. Preferably, such an adhesive is non-irritating, biocompatible and compatible with other components of the adhesive formulation, and biodegradable. Non-limiting examples of adhesives have been described above. The pressure sensitive drug delivery adhesive may be same or different from that used for transdermal drug delivery formulation used in the drug reservoir. In some embodiments, the pressure sensitive adhesive used in the rate-controlling layer is the same as that used in the reservoir layer. In other embodiments, the pressure sensitive adhesive used in the skin-contacting layer (third layer) is the same as that used in the drug reservoir layer.

Preferably, the pressure sensitive adhesives used in the skin-contacting layer comprise polymers selected from the group consisting of acrylates, natural rubbers, and synthetic rubbers. In some embodiments, the adhesives are acrylate adhesives. In some preferred embodiments, the pressure sensitive adhesive is a natural rubber, a synthetic rubber, a polyisoprene, a styrene-isoprene-styrene block (SIS), a styrene-butadiene-styrene block copolymer (SBS), a styrene-butadiene rubber, polyisobutylene rubber, and a combination thereof. In some embodiments, the pressure sensitive adhesive is preferably a SIS and polyisobutylene. One of skill in the art will appreciate that the concentration of adhesives in the device is not so high as to injure or irritate the skin of the subject at removal of the device. In preferred embodiments, the concentration of such an adhesive ranges from 10% to 40%, and preferably from 20% to 40% by w/w of the device.

In some embodiments, the adhesive formulations may be subjected to crosslinking. In a preferred embodiment, the concentration of a cross linking agent ranges from 0.005% and 2% of the adhesive formulation.

The presence of a rate-controlling membrane or rate-controlling layer in the device can change the skin penetration profile of the device compared to a device lacking such a membrane. Thickness of such membranes or layers typically ranges from 20 μm to 120 μm. Preferably, the thickness is 50 μm. Suitable membranes include, without limitation, continuous film membranes prepared from ethylene:vinyl acetate copolymers containing from 0.25% to 25% by weight of vinyl acetate. In some preferred embodiments, continuous film membranes are prepared from ethylene:vinyl acetate copolymers containing 2% to 15% by weight of vinyl acetate.

The concentration of the API in the skin-contacting layer can be higher than that in reservoir layer, lower than that in the reservoir layer or same as that in the reservoir layer. In a preferred embodiment, the concentration of the API in the skin-contacting layer is higher than that in the reservoir layer since the composition in the reservoir will diffuse into the skin contacting layer over time.

Inclusion of different concentrations of controlled release agent in the rate controlling layer, differences in the affinity of the active ingredients in the different layers, and/or the differences in the rate of diffusion across the different layers of the devices can each be used to adjust the rate of delivery of the compositions to a subject. In addition, the relative thickness of the layers may add to the control of rate of drug delivery.

In some embodiments, an adhesive formulation may further include other optional components or additives such as dyes, cross-linkers, tackifiers, transdermal delivery enhancing agents such as permeation enhancers, adhesion promoters, gelling agents, crystallization inhibitors, fillers, emulsifiers, anti-inflammatory agents, antimicrobial agents, moisturizers, preservatives, anti-oxidants, and the like, mixed together in a generally homogeneous mixture.

In at least one embodiment, the adhesive formulation may further comprise one or more additives to prevent crystallization. Crystallization inhibitors useful in the present invention include, without limitation, octyldodecanol, dextrin derivatives, polyethylene glycol (PEG), polypropylene glycol (PPG), mannitol, poloxomers such as poloxomer 407, 188, 401 and 402, and poloxamines, such as poloxamine 904 and 908. Such a crystallization inhibitor may be present in the reservoir or in one or more adhesive layer at a concentration ranging from 0.1% to 10%. In some preferred embodiments, the device comprises crystallization inhibitors at a concentration ranging from 0.5% to 9%, from 1% to 8%, from 2% to 6%, from 3% to 5% w/w of polymer/adhesive.

Polyethylene glycols capable of being included in transdermal drug delivery device such as patch may include a low molecular weight PEG, a high molecular PEG or a combination thereof. Low molecular PEGs, for example, include PEG 200 to PEG 600, preferably, PEG 400 to PEG 600. High molecular PEGs include PEG 2000 to PEG 20,000. In some embodiments, the transdermal patches include PEG 6000 and PEG 20,000. In some embodiments, adhesive compositions include from 2% to 5% PEG 400 to PEG 600. In other embodiments, the adhesive compositions include PEG 2000 to PEG 20, 000 from 1% to 10%, preferably from 2% to 8%, from 4% to 6% and from 3% to 5% by weight of the patch.

The adhesive formulation may further include a tackifier. In some preferred embodiments, the tackifier is a rosin, a rosin derivative, a glycerin ester of a hydrogenated rosin, a pentaerythritol ester of a rosin, a terpene, a modified terpenes, a terpene-phenol resin, an aliphatic resin, a cycloaliphatic resin, and an aromatic resin, a hydrogenated hydrocarbon resin, and a combination thereof. The amount of tackifier ranges from 5% to 70%, preferably ranging from 5% to 60% and more preferably ranging from 10% to 50% by weight based on the total weight of the adhesive formulation.

In other embodiments, the adhesive formulation may include one or more plasticizers. Plasticizers suitable for adhesive formulation have been described above. In some embodiments, liquid paraffin, ISM, diethyl sebacate, and hexyl laurate are preferred, liquid paraffin are more preferred. The concentration of a plasticizer used ranges from 5% to 70% w/w, preferably ranging from 10 to 60% w/w and more preferably ranging from 10% to 50% w/w of the adhesive formulation.

In some embodiments, the adhesive formulation comprises penetration or permeation enhancers disclosed herein, for example, at a w/w concentration from 0.1% to 10%, usually from 2.5% to 7.5%, more usually 5%. Gelling agent such as polyvinylpyrrolidine (PVP) and others disclosed above may also be included in a transdermal patch formulation, for example at a w/w concentration of from 5% to 25%, from 7% to 20%, from 8% to 18%, from 10% to 16%, from 10% to 12%, and from 14%, to 16%.

In some embodiments, the components of a transdermal drug delivery device such as a patch are comprised in a film-forming agent, e.g., PVP, ethylcellulose, and nonionic surfactant, and mixed in a lower alcohol, e.g., ethanol, propanol, or isopropanol, and dried on a hydrophobic surface to form a film that can be adhered to a suitable backing layer.

The device of the present invention may also comprise at least one antimicrobial agent at a concentration ranging from 0.01% to 5%, preferably 0.05% to 2%, more preferably 0.1% to 1% and even more preferably from 0.01% to 0.05%. In a preferred embodiment, the antimicrobial agent is an antibiotic or an antifungal. A preferred antiseptic agent is ethyl para-hydroxybenzoate, propyl para-hydroxybenzoate, butyl para-hydroxybenzoate or a combination thereof.

In some embodiments, the patch can be a patterned patch (e.g., a microneedle patch). For example, in some cases, a silicon wafer with oxide mask can be patterned using standard contact lithographic techniques with thick photoresist and subjected to deep reactive ion etching.

In one aspect, the pharmaceutical compositions of the present invention comprise an agent selected from a rapid-release agent, an immediate-release agent, a slow-release agent, a moderate-release agent, a sustained-release agent, a delayed-release agent and a controlled-release agent. In some embodiments, the sustained or controlled release agent is selected from a group consisting of stearic acid, palmitic acid, glycerol, and PEG esters thereof, Precirol AT05, Imwitor 191, Imwitor 370, Imwitor 375, Myverol 18-06, Caprol ET, Cithrol 2MS, Marosol 183, and combinations thereof.

The patch formulations and adhesive layers are designed to be biodegradable, and when applied to breast tissue or nipple facilitate the delivery of a locally concentrated composition while providing a rapid release, a moderate release, a slow release, a controlled-release, a time release or a sustained drug delivery as desirable during the treatment period. The time that is considered a sufficient can be selected by those skilled in the art with the consideration of the flux rate provide by the device of the present invention and the conditions being treated.

The patch may be of any shape or size suitable for the purpose of the present invention. In one aspect, the patch will applied so as to cover a nipple or an areola. Accordingly, it is contemplated that the in some embodiments, the patch comprises a non-adhesive central zone in contact with nipple. The shape of the patch may be round, oval, rectangular, square, oblong, scalloped or any other suitable shape. The patch may be of any suitable color. In some embodiments, the color of the patch may be neutral (for example, skin or nude colored), black, or white. Generally, a device will have a surface area of 1 cm² to 100 cm² and preferably 5 cm² to 30 cm²

In a preferred embodiment, the patch comprises (i) at least one therapeutic agent selected from the group consisting of a SERM, a SERD, an AI, or a combination thereof, (ii) a fatty acid mixture comprising at least one omega-3 fatty acid, and (iii) at least one vitamin D compound. In some preferred embodiments, the patch comprises endoxifen. In some preferred embodiments, the patch comprises endoxifen, a fatty acid mixture comprising EPA triglyceride, and cholecalciferol.

H. Transpapillary/Intraductal Devices

Pharmaceutical preparations disclosed herein may be delivered to a breast duct of a subject in need thereof. In some embodiments, a pharmaceutical preparation, such as a gel or a solution, more preferably a hydroalcoholic gel or hydroalcoholic solution as disclosed herein, may be delivered to at least one breast duct of a subject. The compositions disclosed herein may be delivered to a single duct or to multiple ducts. In some embodiments, a preparation may be delivered to 1 to 3 ducts, 1 to 4 ducts, 1 to 5 ducts, 1 to 6 ducts, 1 to 8 ducts, 3 to 8 ducts, and 5 to 8 ducts.

The compositions may be delivered locally into a breast duct in any manner suitable for delivery to the duct, for example, by use of a device. In some other embodiments, the methods comprise delivering a composition by transpapillary methods. Methods of transpapillary drug delivery are known in the art [Dave et al. PLos One. 2014, Dec. 29:9(12): e115712; Lee et. al, Intl. J. Pharmaceutics, 387 (2010) 161-166]. A non-limiting example of such a device is one which delivers compositions to a breast duct via the nipple papilla as described in patent application No. PCT/US15/10808, which is herein incorporated by reference in its entirety. In some embodiments, the preparation is forced into a breast duct under positive pressure. In some preferred embodiments, the preparation is contained in a treatment chamber of a device and is delivered to a subject by transpapillary delivery method by contacting the preparation with a nipple or an areola of a breast and applying positive pressure on the preparation forcing the preparation in to a breast duct.

In other embodiments, a preparation may be delivered intraductally using a breast duct device. Non-limiting examples of breast duct devices used for delivery into a duct include syringe and needle, micro-needles, catheters, micro-catheters, or any other device that can be suitably inserted into a breast duct, for example, via a nipple. Some examples of breast duct devices that are useful in delivering a composition are described in U.S. Pat. No. 7,384,418; U.S. Pat. No. 6,689,070; and U.S. Pat. No. 6,413,228, each of which is incorporated by reference in its entirety in this application. In some embodiments, a breast duct may be infused with a composition described herein by placing a breast duct device (e.g., a microcatheter) into the nipple and injecting the preparation into the breast duct. In other embodiments, the preparation may be administered to a subject using an implanted or indwelling breast duct device. Accordingly, the compositions may be delivered to a breast duct of a subject using an indwelling reservoir capable of being placed in the breast duct. In some embodiments, the device may have a line or tube connected to the reservoir to reload the indwelling reservoir when empty, or to provide retrieval of the indwelling reservoir from the breast duct. For example, 5 to 8 ducts may be dilated, cannulated and a composition maybe delivered into the ducts with a suitable indwelling device which includes, without limitation, indwelling catheters, beads, microbeads, chips, or any other form of device suitable for drug delivery. The device may be any size suitable for delivery of a composition disclosed herein. In some embodiments, the breast duct device is biodegradable. In other embodiments, the breast duct device is non-allergenic. In a particularly preferred embodiment, the breast duct device is a catheter, a microcather, or a nanocatheter. The drug delivery into a duct may be rapid or sustained over time.

I. Oral Dosage Forms

In another aspect of the invention, pharmaceutical compositions are formulated for oral delivery. An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. In some embodiments, the oral formulation is a capsule, a caplet, or a tablet. When the oral dosage form is a tablet, the tablets for example may be disintegrating tablets, fast-dissolving tablets, effervescent tablets, fast melt tablets, and/or mini-tablets.

When the dosage form is a capsule, in at least one preferred embodiment, the capsule is a seamless capsule. In other preferred embodiments, the capsule is a hard capsule or a soft capsule. In a more preferred embodiment, the capsule is a gelatin capsule, gelatin-free capsule, a “cap-in-cap” capsule, alginate capsule, hydroxypropylmethyl cellulose (HPMC) capsule, a polyvinyl alcohol (PVA) capsule, or a starch capsule.

An example of a “cap-in-cap” capsule is a DuoCap™. DuoCap involves specialist filling techniques using custom designed filling equipment that allows the insertion of a pre-filled, smaller capsule into a larger liquid-filled capsule. The smaller, inner capsule may contain either a liquid or a semi-solid formulation and, according to the formulation or product requirements, either or both capsules may be of gelatin or HPMC composition and can be coated, if necessary. DuoCap™ has been successfully commercialized by Encap and is suitable for the purposes of the present invention.

In an aspect, the alginate capsule comprises an alginate that is an M-alginate, a G-alginate, or a combination thereof. In one embodiment, the alginate comprises from 1% to 80% by weight with respect to the total weight of the shell.

In an embodiment, the capsule is a hard gelatin capsule or a soft gelatin capsule. In one preferred embodiment, the capsule is a soft gelatin capsule. In another preferred embodiment, the capsule is a hard gelatin capsule.

In one aspect, gelatin ranges from 1% to 80% by weight with respect to the total weight of the shell. In another aspect, the gelatin Bloom strength in the soft gelatin capsule is typically 150 to 200 Blooms (or grams). The Bloom strength in the hard gelatin capsule is adjusted higher than that of the soft gelatin capsule, 200-300 Blooms. Viscosity of the a hard capsule is preferably from 44 to 60 mPa while the viscosity of a soft gel capsule is preferably from 2.8 to 4.5 mPa, depending on the type of gelatin used.

The gelatin may be from an animal or a non-animal source. In an aspect, the gelatin capsule comprises a gelatin selected from type A gelatin, type B gelatin, and mixtures thereof. Gelatin is produced by destruction of secondary and to some extent higher structures of collagen. Two common processes by which collagen is processed to form gelatin include the acidic and the alkaline pre-treatment followed by extractions. Type A gelatin is extracted from the animal skin or hide and soaked in acid for 10-30 hours depending on the nature of the collagen stock. Type B gelatin is derived from collagen raw materials subjected to alkaline pre-treatment and left in liming pits for 3-10 weeks depending on the nature of the stock and ambient temperature.

In some preferred embodiments, the capsules are made of non-animal materials or plant materials. In one aspect, a non-animal capsule comprises starch which is formulated with conventional plasticizers such as glycerol, sorbitol etc., (10% to 60% w/w of dry shell) and water to form a molten mass that can be extruded to set within less than 20 seconds producing a mechanically strong, elastic film on temperature-controlled casting drums. Sealing is performed at temperatures between 25° C. and 80° C. Starch may be obtained from any source, for example vegetables such as potato. In some embodiments, the amylopectin content is 50% w/w. Size of the starch capsules may be number 0, 1, 2, 3 and 4-USP 23 and NF18. Examples of starch capsule include pullulan capsules. Other starch capsules commercially available include VegiCaps™ manufactured by Catalent.

Capsules may be amenable to use in capsule filling machine instrumentation for the filling of pharmaceutical drugs and capsule fill phase formulations. Accordingly, a capsule of this invention comprises a shell and a fill phase. In some embodiments, the capsule may be an orally dissolving capsule wherein the capsule disintegrates in saliva of the subject with no need for chewing or drinking liquids to ingest the capsule. In other embodiments, the capsule may be a time-release or controlled release capsule with at least one protective coating such as an enteric coating to permit the capsule to pass through the stomach with very little release of the therapeutic agents in the stomach and become dissolved in the upper intestines. In some embodiments, the shell comprises at least one strengthening agent. In some preferred embodiments, the strengthening agent is polydextrose or poly-L-ornithine.

Accordingly, the shell of the capsule of the present invention may comprise at least one coating. Such coating can delay the release of active pharmaceutical ingredients and act as a protective barrier layer or both. For example, the at least one coating may allow the oral dosage form to pass through the stomach without being subjected to stomach acid or digestive juices to provide for delayed release outside the stomach of the active pharmaceutical ingredients of a composition described herein, namely, at the least one therapeutic agent and/or the fatty acid mixture comprising the at least one omega-3 fatty acid, and/or the at least one vitamin D compound. Accordingly, in some embodiments, the dosage forms of the present invention includes capsules that are delayed-release, modified-release, time-release, controlled release-release, extended-release, and sustained-release dosage forms.

In some embodiments, the capsules release in stomach less than 30% of the total API, such as <10%, <15%, <20%, and <25% and the rest in the small intestine. The present invention also encompasses embodiments wherein each API of the composition (the at least one therapeutic agent, the fatty acid mixture comprising the at least one omega-3 fatty acid, and the at least one vitamin D compound) may be released at different times and locations during its passage through the gastrointestinal tract. In some embodiments, the capsules may be single-phase capsule. In other embodiments, the capsules may be multi-phase capsule.

In some embodiments, the at least one coating is selected from an enteric coating, a sub-layer, a top-layer, and a combination thereof. The term “sub-layer” as used herein, means a coating layer located between a capsule wall (e.g., shell wall) or the tablet surface and an enteric coating. The “top-layer” as used herein means a coating layer over an enteric coating covering the capsule shell.

In some embodiments of the present invention, the shell of the capsule comprises at least one enteric coating. In other embodiments, the gelatin capsule comprises at least one enteric coating and at least one top-layer above the enteric coating. In another embodiment, the gelatin capsule comprises at least one enteric coating and at least one sub-layer between a capsule wall and the enteric coating. In yet another embodiment, the gelatin capsule comprising the at least one enteric coating and at least one sub-layer between a capsule wall and the enteric coating further comprises at least one top-layer above the enteric coating.

In one aspect, the at least one sub-layer comprises a sealant. Suitable sealants comprise, for example, permeable or soluble agents such as HPMC, hydroxypropyl cellulose, hydroxypropyl ethyl cellulose, guar gum and xanthum gum. Other agents can be added to improve processability of the sealant or the barrier layer. Such agents include talc, colloidal silica, polyvinyl alcohol, titanium dioxide, micronized silica, fumed silica, glycerol monostearate, magnesium trisilicate, and magnesium stearate or a mixture thereof. The sealant or the barrier layer can be applied from solution (e.g., aqueous) or suspension using any known means, such as fluidized bed coater (e.g., Wurster coating) or pan coating system. Suitable sealants or barriers include, for example, Opadry® products from Colorcon. In one embodiment, the at least one sub-layer and/or the at least one top-layer comprises HPMC.

Non-limiting examples of coating materials contemplated for the present invention include gelatin, film-forming agents, polymers, and copolymers. The chemical compositions of the sub-layers and top-layers may vary depending upon the overall composition of the capsule. Polymers used herein may be acid-insoluble or acid-soluble. For delayed-release capsules for example, acid-insoluble polymers are preferred as a coating material, for example, for enteric coating. Non-limiting examples of acid-insoluble polymers are known in the art. Non-limiting examples have been described above. Additional materials suitable for the at least one coating include pharmaceutically acceptable acidic compounds that may not dissolve at low pH in the stomach but may dissolve at higher pH in the intestines. In some preferred embodiments, polymers and copolymers include without limitation, acrylate-based polymers and copolymers. In some preferred embodiments, methacrylic acid, copolymers between methyl methacrylate, copolymers between methacrylic acid and methyl acrylate, copolymers between metacrylic acid and ethyl methacrylate, and copolymers between metacrylic acid and ethyl acrylate), and polysaccharides and/or cellulose-based polymers and copolymers (cellulose acetate phathalate, HPMC phthalate, and HPMC acetate succinate are preferred. In other embodiments, a preferred polymer includes polyvinyl acetate phathalate. The acid-insoluble polymer or copolymer is present in an amount ranging from 8% to 20% by weight of the wet gelatin mass.

The at least one coating may be pH-independent or pH-dependent. In one aspect of the present invention, the at least one coating is pH-independent. Coatings with pH-independent profiles generally erode or dissolve away after a pre-determined period. The period is generally proportional to the thickness of the coating. In at least one embodiment, the at least one coating comprises a pH independent polymer. EUDRAGIT® RS 30D is a preferred polymer for the at least one coating. In another aspect, the at least one coating is pH-dependent. Coatings with the pH-dependent profiles can generally maintained their integrity while in the acid pH of the stomach, but will erode or dissolve away after entry into the more basic environment of the jejunum, the upper intestine. In some preferred embodiments, the at least one coating comprises a pH dependent anionic polymer. EUDRAGIT® L30D 55 is preferred. In some embodiments, the at least one coating comprises a water soluble polymer. PVP is preferred. In some embodiments, the at least one coating is insoluble at pH below 5 and soluble at a pH above 6.

In some embodiments, typical materials for enteric layers, sub-layers and top-layers disclosed herein include film-forming agents. Non-limiting examples of film-forming agents have been described above. In at least one embodiment, the film-forming agent comprises HPMC. The composition ratio between a film-forming agent and a polymer is adjusted so that a gel mass can be made into soft capsules. The ratio of film-forming agent to polymer may range from 15:85 to 45:55 w/w of the shell. In a preferred embodiment, the weight ratio of film-forming agent:acid-insoluble polymer is 15%:50%.

The amount of coating material or thickness of the at least one coating may vary depending upon the chemical composition and number of different coating layers, and chemical composition, size, and shape of the capsule. The coating is of sufficient thickness to prevent substantial release of the at least one therapeutic agent, the fatty acid mixture comprising the at least one omega-3 fatty acid, and the at least one vitamin D compound, but does not contribute significantly to the size of the capsule or the tablet. In some embodiments, the thickness of the at least one coating ranges from 10 microns to 2 mm, preferably from 20 microns to 1 mm, more preferably from 5 microns to 0.5 mm. In some embodiments, the at least one coating comprising from 1% to 50% of the dry capsule shells material (e.g., gelatin). In some embodiments, weight of a shell comprising the at least one coating ranges from 10% to 20% of the final weight of a capsule.

The at least one coating may comprise at least one plasticizer selected from a group which includes, without limitation, triethyl citrate, triacetin, polyethylene glycols, polypropylene glycol, phthalates, sorbitol, and glycerin or mixtures thereof. In one embodiment, the at least one plasticizer is sorbitol or glycerol. The amount of plasticizer may vary depending upon the chemical composition of the at least one coating and the chemical composition and size of the capsule, and/or the caplet and/or the tablet. In some embodiment, for example, the amount of plasticizer ranges from 10% to 60% by weight of the at least one coating, such as from 10% to 50%, from 20% to 40%, from 30% to 35%. In other embodiments, the ratio of plasticizer to polymer ranges from 10% to 50%, from 20% to 40%, and from 30% to 35%.

In some embodiments, the at least one coating comprises the at least one therapeutic agent, (for e.g., a SERM, a SERD, an AI, or a combination thereof). In some preferred embodiments, the at least one therapeutic agent is dissolved, dispersed, microencapsulated, nano-encapsulated, or otherwise contained in the film-forming agent in the at least one coating of the capsule shell. The shell comprises 1% to 40% of the at least one therapeutic agent. In some preferred embodiments, the shell comprises 0.5 mg, 1 mg, 1.5 mg, 2 mg, 5 mg, 10 mg, 50 mg, and 100 mg of the at least one therapeutic agent. In at least one preferred embodiment, the shell comprises 0.5 mg to 1 mg of anastrozole. In another embodiment, the at least one coating comprises at least one therapeutic agent and at least one additional medication.

In at least one embodiment, the shell comprises a gelatin, a plasticizer, and water or a buffer. In another embodiment, the shell comprises a plant polysaccharide, polysaccharide derivatives or combinations thereof. In a preferred embodiment, the shell comprises a starch, a carrageenan or a combination thereof. In a more preferred embodiment, the shell comprises a film coat formulation comprising an Eudragit L30D-55 dispersion, PEG-400, Talc, an antifoaming agent, and water or a buffer.

Antifoaming agents are useful in the present invention to prevent bloating and include antifoaming agents that are oil-based, powder-based, silicone based, EO/PO based, or alkyl polyacrylates. Example of silicone based antifoaming agent includes, without limitation, simethicone. When the antifoaming agent is used, it may be used as an oil-based emulsion.

In still another aspect, the at least one coating may optionally comprise a colorant, an opacifier, a sweetener, and antioxidant, an anti-adhesion agent, a solubilizer, a dispersion agent or a combination thereof.

When the oral dosage form is a rapidly dissolving form, the shell further comprises a dissolution enhancing agent to enhance the disintegration, and/or increase the rate of dissolution, of the film, or the capsule in the oral cavity or in water. The dissolution enhancing agent may be selected from maltose, lactose, sorbitol, gluconic acid lactone, mannitol, xylitol, maltitol, and isomalt. In one aspect, the dissolution enhancing agent is present from 0.1% to 35% by weight of the capsule shell solids. In some embodiments, 0.1% to 1% sodium lauryl sulfate may be added to the fill phase to enhance penetration of water into the capsule to speed dissolution.

In some embodiments, the shell comprises a wetting agent. In some embodiments, from 0.1% to 15%, 0.5% to 10%, from 1% to 5% or from 1.5% to 3% by weight of a wetting agent is included in the shell. In a preferred embodiment, the wetting agent is Solutol. In another preferred embodiment, the wetting agent is solutol and the dissolution enhancing agent is sorbitol. In yet another preferred embodiment, the wetting agent is Solutol and the plasticizer is glycol. In yet another preferred embodiment, the wetting agent is Solutol and the strengthening agent is polydextrose.

In some embodiments, the capsule shell further comprises a strengthening agent. The strengthening agent is selected from the group consisting of polydextrose, cellulose, cellulose derivatives, maltodextrin, guar gum, gelatin, alginates, and gum Arabic. The strengthening agent is present from 0.1% to 20% by w/w of the shell. In a preferred embodiment, the strengthening agent is polydextrose.

In still another aspect, the fill phase of the capsule comprises a solid, a semi-solid, a liquid or solution fill, a suspension, or a combination thereof. In some embodiments, a fill phase is selected from the group consisting of oil-based fills, alcoholic fills, pastes, powders, and a combination thereof. In some embodiments, the fill phase comprises an excipient. In other embodiments, the fill phase further comprises a vehicle, a co-solvent, a permeation enhancer, an absorption enhancer, a surfactant, a gelling agent, a neutralizing agent, or a combination thereof. In one embodiment the vehicle is a lipophilic, a hydrophilic or a mixed vehicle.

In a preferred embodiment, the fill phase comprises a liquid fill. In some embodiments, the fill phase comprises a fatty acid mixture comprising at least one omega-3 fatty acid. In other embodiments, the fill phase comprising a fatty acid mixture comprising at least one omega-3 fatty acid further comprises at least one vitamin D compound, and optionally, at least one additional medication. In some embodiments, the fatty acid mixture is a fatty acid oil mixture. Accordingly, in some preferred embodiments, the fill phase comprises fish oil. In a preferred embodiment the fish oil is an anchovy fish oil, a tuna fish oil, or a cod fish oil. In some embodiments, the fish oil may be enriched with omega-3 fatty acids. In other embodiments, the fill phase comprising fish oil enriched with omega-3 fatty acid may further comprise at least one vitamin D compound as described above.

Self-emulsifying lipid-based formulations can enhance bioavailability of an API and minimize absorption variability with little to no “food effect.” Thus, in other embodiments, a fill phase may comprise at least one emulsion comprising at least one oily phase, the at least one oily phase further comprising a fatty acid mixture and at least one surfactant. In yet another embodiment, the at least one oily phase further comprises at least one vitamin D compound, and optionally at least one additional medication. In still another embodiment, the oily phase comprises a co-surfactant. In at least one embodiment, the fatty acid mixture comprising the at least one omega-3 fatty acid ranges from 50% to 90%, from 55% to 85%, from 60% to 80%, from 65% to 75%, and from 70% to 95% by weight of the emulsion.

In some embodiments, the emulsion is an oil-in-water emulsion, a water-in-oil emulsion, or a water-in-oil-in-water emulsion. In some preferred embodiments, the emulsion is an oil-in-water emulsion. The emulsions may include emulsifiers that are hydrophilic or lipophilic. In some embodiments, the oil-in-water emulsion is selected from LipovenoesR, LipovenoesR 10% PLR, StructolipidR, and OmegavenR.

In some embodiments, the at least one emulsion further comprises from 0.1% to 3% surfactant by weight and from 0.1% to 6% of at least one gelling agent by weight, each with respect to the total weight of the at least one emulsion. Surfactants that may be used have been described above. In a preferred embodiment, the surfactant is Capmul. In some embodiments, the gelling agent is HPMC or CMC. The viscosity of the fill phase is any viscosity suitable for encapsulation into capsules and may be adjusted by varying the amount of gelling agent in the fill phase.

In one aspect, at least one therapeutic agent is dissolved, dispersed or suspended in the fill phase. In some embodiments, the at least one therapeutic agent is partially or wholly dissolved, dispersed or suspended in the fatty acid mixture comprising the at least one omega-3 fatty acid. In other embodiments, the at least one therapeutic agent is partially or wholly dissolved, dispersed or suspended in the fatty acid mixture comprising the at least one omega-3 fatty acid and the at least one vitamin D compound. In another embodiment, the at least one therapeutic agent is comprised in a preformed solid dosage form encapsulated within the fatty acid mixture comprising the at least one omega-3 fatty acid and the at least one vitamin D compound. In yet another embodiment, the foregoing preformed solid dosage form comprising the at least one therapeutic agent (such as a SERD, a SERM, an AI, or a combination thereof) encapsulated within the fatty acid mixture comprising the at least one omega-3 fatty further comprises the at least one vitamin D compound, at least one additional medication or a combination thereof.

A fill phase may optionally comprise at least one excipient useful for enhancing bioavailability of an API, which may be present in the shell or in the fill phase. The bioavailability of poorly soluble drugs, such as fulvestrant, cis-tamoxifen, endoxifen, anastrozole, and 4-OHT, can often be enhanced from lipid-based formulation filled into soft capsules that are digested in-vivo by lipolysis. In some embodiments, the fill phase further comprises an absorption enhancer, such as a bile salt or a chelating agent.

Optionally, polymers such as cellulose polymers (methylcellulose), mono- and diglycerides of Capryl/Capric acids, caprylocapryl macroglycerides, polysorbate 80 and/or sodium alginate may be added to the fill phase to control the rate of release of API. One of skill in the art will recognize that the rate of release is delayed as the proportion of polymers or alginates in the fill phase is increased relative to water soluble ingredients such as lactose.

The oral dosage forms may also comprise colorants, flavoring agents, preservatives and other additives.

In some preferred embodiments, a fill phase may comprise other excipients, such as mineral oil and waxes, (for e.g., paraffin wax), polyethylene glycols (for e.g., PEG 400 to PEG 600), solvents (for e.g., dimethyl isosorbide, diethylene glycol monoethyl ether), glycerin, and polyvinylpyrrolidine (PVP)).

In some embodiments, the fill phase comprises water or alcohol ranging from 0% to 10% w/w for increasing solubility of the API, glycerin ranging from 1% to 4% to retard the migration of glycerin out of the shell into the fill, PVP ranging from 0% to 10% w/w used in combination with PEG to increase API solubility and improve stability by inhibiting drug crystallization.

Accordingly, a preferred embodiment of the present invention provides a capsule, a caplet or a tablet comprising: at least one therapeutic agent, a fatty acid mixture comprising at least one omega-3 fatty acid, and at least one vitamin D compound. In some embodiments, the oral dosage form is a capsule comprising a fill phase comprising a fatty acid mixture comprising at least one omega-3 fatty acid and at least one vitamin D compound encapsulated in a shell comprising at least one therapeutic agent.

In yet another embodiment, the oral dosage form is a soft capsule or a hard capsule comprising: (i) a fill phase comprising a fatty acid mixture comprising EPA triglyceride or phospholipid, cholecalciferol, and optionally an excipient, and (ii) a shell comprising a film-forming agent comprising at least one therapeutic agent selected from a group consisting of a SERM, a SERD, an AI, and a combination thereof, wherein concentration of the at least one therapeutic agent ranges from 0.01 mg to 500 mg. In some preferred embodiments, the concentration of the at least one therapeutic agent ranges from 0.1 mg to 100 mg. In yet another embodiment, the oral dosage form is a soft capsule comprising 40 mg of fulvestrant. In still another embodiment, the oral preparation is a soft capsule comprising 0.5 mg to 10 mg of anastrozole.

In some embodiments, oral pharmaceutical compositions comprise: at least one therapeutic agent; a fatty acid mixture comprising at least one omega-3 fatty acid; and at least one vitamin D compound. In some embodiments, the composition is a capsule, a caplet, and a tablet. In some embodiments, the capsule is a gelatin capsule, gelatin-free capsule, a cap-in-cap capsule, alginate capsule, an HPMC capsule, a PVA capsule, and a seamless capsule. In some embodiments, the capsule is a hard capsule or a soft capsule.

J. Capsule Preparations

Capsule shells may be made using any of the methods known in the art, for example, by using the plate process, the rotary die process, the reciprocatory die process, the Norton capsule machines and Accogel capsule machines (McGuffy, Irena, Softgel Technology as a Lipid-Based Delivery Tool for Bioavailability Enhancement, Catalent Pharma Solutions, Somerset, N.J., March 2011). Exemplary manufacturers of soft and hard gelatin capsules include Catalent Pharma Solutions, Somerset N.J., Pharmagel Engineering spa, Lodi, Italy, and Soft Gel Technologies Inc., Commerce, Calif., CapsuGel, Inc., Morristown, N.J., Eli Lilly, Ind. Manufactures of HPMC capsule shells include Shionogi Qualicaps (QUALI-V), Vegicaps (Catalent).

A brief method of making a soft gelatin capsule shell follows.

A desired amount of gelatin (150 Blooms for a soft shell) is mixed with water. A plasticizer such as sorbitol or glycerin or a combination of both, is added to the mix of gelatin and water and melted in a heated tank (gelatin melter) typically for about 3 hours until gelatin turns into a molten liquid gel mass. Optionally, opacifier, a coloring agent, or flavor mask may be added to the mix prior to melting the gelatin. The gel mass is taken in a closed clean environment for cooling and storage. As sheets of gelatin are formed, two flat ribbons may be manufactured using twin set of rotary dies which contain recesses in the desired size and shape. The rotary dies cut out the ribbons into a two-dimensional shape, and form a seal around the outside. At the same time, a pump may deliver a precise dose of fill phase into the shells through a nozzle incorporated into a filling wedge, whose tip sits between the two ribbons in between two die pockets at the point of cut out. The wedge is heated to facilitate the sealing process. The wedge injection causes the two flat ribbons to expand into the die pockets, giving rise to the three-dimensional finished product. After encapsulation, the soft gel capsules are dried for two days to two weeks depending on the product and polished.

The capsule shell is provided with at least one coating. The coating may be provided in any manner suitable, for example, by passing through fluidized air beds or horizontal drums, spraying or dipping in a coating onto a surface of a capsule shell, and the like. As a non-limiting example, a desired amount anastrozole may be mixed with alcohol (e.g., ethanol or isopropanol) and a film-forming agent (e.g., HPMC) and stirred until anastrozole is completely dissolved in the film-forming agent such that each capsule will deliver a fixed predetermined amount of anastrozole to a subject, for e.g., 0.5 mg or 1 mg per capsule. The film-forming agent comprising anastrozole may be next sprayed onto the surface of the capsule shell. Alternatively, the capsule shell may be dipped into film-forming agent comprising anastrozole. The capsule is then dried, polished, and stored in an appropriate dispenser, such as a light and airtight bottle.

To prepare an exemplary fill phase of a gelatin capsule, ethanol is added to isopropanol to prepare alcohol mixture and stirred. A fatty acid mixture comprising at least one omega-3 fatty acid in a triglyceride form, for example EPA triglyceride, is added to an ethanol/isopropanol mixture while stirring under nitrogen. A surfactant such as glycerol may optionally be added to the mixture. Next, at least one vitamin D compound, Cholcalciferol, is slowly added to the fatty acid mixture comprising the omega-3 fatty acid triglyceride until completely dissolved. Fish oil is added in sufficient amounts to a desired final weight of the fill phase formulation. The solution is sterilized by filtration using typically one or two filters of 0.2 μm porosity. The sterile filtrate is kept under N₂ overlay as it is filled into a capsule shell.

In another embodiment, a desired amount of cholecalciferol is added to commercially available purified omega-3 fish oil and filled into a capsule shell prepared as described above. Optionally, anti-oxidant, such as alpha-tocopherol, may be added to the purified omega-3 fatty acid triglyceride fish oil.

As another example, a soft gelatin capsule may be prepared as described above. A fill phase may be prepared as described. To the fill phase, anastrozole dissolved in an alcoholic solvent such as alcohol such as ethanol and isopropyl alcohol, may be added to the fatty acid mixture comprising at least one omega-3 fatty acid, and at least one vitamin D compound such as cholecalciferol.

Accordingly, in one aspect, a method of preparing a pharmaceutical preparation is provided comprising preparing a mixture of: (a) at least one therapeutic agent: (b) a fatty acid mixture comprising at least one omega-3 fatty acid; (c) at least one vitamin D compound; (d) and optionally, an excipient.

In some embodiments, the method comprises dissolving, dispersing or suspending the at least one therapeutic agent in the fill phase of oral dosage form. In some preferred embodiments, method comprises preparing the mixture in an oral dosage form. In other embodiments, the method comprises providing the at least one therapeutic agent comprised in a shell. In some embodiments, the method comprises providing a plurality of therapeutic agents. In other embodiments, method comprises providing a fatty acid mixture comprising a plurality of omega-3 fatty acids. In some embodiment, the method comprises a composition in an oral dosage form selected from a group consisting of a capsule, caplet and a tablet. In a preferred embodiment, the oral dosage form is a capsule. In still another embodiment, the oral dosage form is a soft capsule or a hard capsule. In some preferred embodiments, the at least one therapeutic agent is a SERD, a SERM, an AI or a combination thereof. cis-tamoxifen, desmethyltamoxifen, endoxifen, 4-OHT, fulvestrant and anastrozole are preferred.

In one aspect, the method comprises a preparation which is in a form selected from a group consisting of an immediate-release, a rapid-release, a slow-release, a moderate-release, a sustained-release, a timed-release, a delayed-release, and a controlled-release form. In a preferred embodiment, the method comprises preparations, wherein the preparation is in sustained-release form.

In some embodiments, the method comprises providing at least one coating to the capsule shell. In some preferred embodiments, the at least one coating is an enteric coating, a sub-layer, a top-layer, or a combination thereof.

In other embodiments, the method comprises providing at least one coating comprising at least one material selected form gelatin, alginate, film-forming agents, polymers, and copolymers. In yet other embodiments, the method comprises providing at least one plasticizer in the at least one coating. The plasticizer in the coating is selected from the group consisting of triethyl citrate, polyethylene glycol, propylene glycol, phthalates, sorbitol, glycerin, and mixtures thereof. In some embodiments, the method provides at least one coating comprising a sealant. In other embodiments, the method provides at least one coating comprising a permeation enhancer. In other embodiments, the method provides at least one coating comprising a film-forming agent comprising Eudragit L30D Dispersion, PEG400, Talc, Simethicon emulsion, and water. In other embodiments, the film-forming agent is from 5% to 15%, from 10% to 15%, from 11% to 14%, and from 10% to 13% by weight of the composition. In other embodiments, the film-forming agent comprises the at least one therapeutic agent. In still more embodiments, the method provides a film-forming agent comprising a plurality of therapeutic agents.

In some embodiments, shell is comprised of gelatin, alginate, cellulose, starch, PVP copolymer or a mixture thereof. In a preferred embodiment, the capsule is hard capsule or a soft capsule. In another preferred embodiment, the capsule is a soft gelatin capsule. In yet another preferred embodiment, the capsule is a HPMC capsule or a starch capsule. In another embodiment of the method, starch and carrageenan or a combination of both is used to prepare the capsule shell.

In some embodiments, the method provides that the soft capsule is prepared using a plate process, a rotary die process, or a reciprocating die process. In some embodiments, the method provides ribbons for preparation of shell ranging in thickness from 0.015 inches to 0.050 inches. In a preferred embodiment, the thickness is 0.020 inches. The moisture content of the shell compositions can be from 2% to 0% and from 4% to 8%. In a preferred embodiment, the moisture content is 8%.

In other embodiments, the method provides that a fill phase comprises a vehicle, a mineral oil, a wax matrix, a bulking agent, a lipophilic emulsifier, and a hydrophilic emulsifier, or a combination thereof. In some embodiments, method provides that the fill phase may optionally comprise a neutralizing agent, an absorption agent, a gelling agent, or a combination thereof.

In yet other embodiments, the method provides that the fill phase may comprise at least one additional medication. In some preferred embodiments, the method provides that the at least one additional medication. The anti-cancer antibody may be selected from the group consisting of nimotuxumab, trastuzumab (Herceptin™), Alemtuzumab (CAMPATH™), Bevacizumab (Avastin™), Brentuximab, vedotin (Adcetris™), Cetuximab (Erbitux), Gemtuzumab (Mylotarg), Ipilimumab (MDX-101/Yervoy), Ofatumumab (Arzerra), Panitumumab (Vectibix), Rituximab (Rituxin, Mabthera), and Tositumomab (Bexxar). Trastuzumab is particularly preferred.

In some preferred embodiments, a method of preparing a pharmaceutical preparation is provided comprising the steps of: (a) providing an amount of at least one therapeutic agent; (b) providing an amount of a fatty acid mixture comprising at least one omega-3 fatty acid; (c) providing an amount of at least one vitamin D compound; (d) providing at least one excipient; (e) combining the fatty acid mixture comprising at least one omega-3 fatty acid, the at least one vitamin D compound, and the at least one excipient, thereby forming a fill phase; and (t) encapsulating the fill phase in a shell, wherein the at least one therapeutic agent is comprised in the shell. Pharmaceutical preparations prepared by such methods comprise capsules, caplets, and tablets.

In at least one preferred embodiment, a method of preparing a pharmaceutical composition is provided comprising the steps of: (a) providing an amount to a fatty acid oil mixture comprising EPA and DHA ranging from 1:10 to 10:1 w/w; (b) providing at least one vitamin D compound in the fatty acid oil mixture comprising the EPA and DHA; (c) encapsulating the fatty acid oil mixture in a shell; (d) providing the shell with a coating; and (e) providing an amount of anastrozole in the coating of the shell.

Quantity of each component of the formulation is chosen according to the required formulation specification, examples are described herein. For example, quantities are added of each component to prepare a formulation which comprises: 60% w/v of a fatty acid mixture comprising EPA triglyceride, 2000 IU of cholecalciferol, 40% w/v mixture of ethanol and isopropanol, 0.5 mg of anastrozole for each finished soft capsule, and remaining amount is fish oil.

In some embodiments, capsules for oral delivery comprise a shell comprising 0.1 mg to 500 mg of a SERM, a SERD, an AI, or a combination thereof; and a fill phase comprising 20% to 60% of a fatty acid mixture comprising at least one omega-3 fatty acid triglyceride or phospholipid; and 10 IU to 6000 IU of at least one vitamin D compound.

In some embodiments, soft gelatin capsules comprise a shell comprising 0.5 mg or 1 mg of anastrozole; a fill phase comprising (a) 60% of a fatty acid mixture comprising at least 99% EPA triglyceride or phospholipid; and (b) 400 IU of cholecalciferol; and a sufficient amount of fish oil.

K. Kits

The present invention also relates to kits comprising the pharmaceutical compositions disclosed herein. In one embodiment, a kit comprises: a first pharmaceutical composition comprising an amount of at least one therapeutic agent and a pharmaceutically acceptable carrier thereof: a second pharmaceutical composition comprising an amount of a fatty acid mixture comprising at least one omega-3 fatty acid; a third pharmaceutical composition comprising an amount of at least one vitamin D compound and a pharmaceutically acceptable carrier thereof; and instructions for use of the first, second and third pharmaceutical compositions together to treat a subject in need thereof.

In another embodiment, a kit comprises: a first pharmaceutical composition comprising an amount of at least one therapeutic agent and a pharmaceutically acceptable carrier thereof; a second pharmaceutical composition comprising an amount of a fatty acid mixture comprising at least one omega-3 fatty acid and an amount of at least one vitamin D compound; and instructions for use of the first and second pharmaceutical compositions together to treat a subject in need thereof.

In yet another embodiment, a kit comprises: a first pharmaceutical composition comprising an amount of at least one therapeutic agent and an amount of a fatty acid mixture comprising at least one omega-3 fatty acid; a second pharmaceutical composition comprising an amount of at least one vitamin D compound and a pharmaceutically acceptable carrier thereof; and instructions for use of the first and second pharmaceutical compositions together to treat a subject in need thereof.

In still another embodiment, a kit comprises: a single pharmaceutical composition comprising an amount of at least one therapeutic agent, an amount of a fatty acid mixture comprising at least one omega-3 fatty acid, and an amount of at least one vitamin D compound and a pharmaceutically acceptable carrier; and instructions for use of the pharmaceutical composition to treat a subject in risk for or having a breast condition.

In another aspect, kits disclosed herein optionally comprise a pharmaceutical composition comprising at least one additional medication and a pharmaceutically acceptable carrier thereof. Examples of such an additional medication have been disclosed herein.

In yet another aspect, the kits disclosed herein optionally comprise a device for delivering the pharmaceutical compositions therein to the subject in risk for or having a breast condition. Any suitable device may be used for delivering the pharmaceutical compositions disclosed herein. Particularly suited for use are devices described in U.S. Pat. Nos. 5,798,266, 6,689,073, and 6,887,210.

A preferred embodiment, a kit suitable for treatment of a subject in need thereof comprises: a device for delivering a pharmaceutical composition to a breast duct of the subject, the device comprising a unit for holding the composition to be delivered to the breast duct, the unit being sized and configured to be positioned and supported on a nipple, and an elongated member for delivering the composition from the unit to the breast duct, the elongated member being in communication with the unit, being sized for positioning within the breast duct, and having a distal terminal end for positioning within the breast duct, said distal end having an atraumatic tip; a pharmaceutical composition comprising at least one therapeutic agent, a fatty acid mixture comprising at least one omega-3 fatty acid, and at least one vitamin D compound; and instructions for use of the device together with the pharmaceutical composition.

In some embodiments, the invention provides a dose, a unit dose, or multiple dose of the pharmaceutical dose package. In some embodiments, the packaging reflects a dosing regimen or schedule of application, such as twice daily, daily, weekly, twice weekly, biweekly, monthly, quarterly, 6 monthly, or yearly application. Advantageously, such packaging of the pharmaceutical composition facilitates accurate application of an amount of the composition such as a therapeutically effective amount and will include a single dose applicator or metered dose applicator.

In one embodiment, the pharmaceutical composition is preloaded in the unit for holding the composition in the device.

The compositions disclosed herein can be packaged in any suitable container to suit its viscosity and intended use. The invention accordingly also provides a closed container containing a composition disclosed herein.

### V. Treatment Methods and Regimen

The present invention also provides methods of treating a subject in risk for or having an estrogen-related disorder or a breast disorder by administering to the subject a pharmaceutical composition that comprises at least one therapeutic agent, a fatty acid mixture comprising at least one omega-3 fatty acid, and at least one vitamin D compound. The present invention particularly encompasses methods of prevention of breast disorders and/or estrogen-related disorder in subjects at risk for developing and/or recurrence of such disorders, especially in high risk women and women who have at least two family members with such disorders.

In methods described herein, subjects may be administered pharmaceutical compositions and preparations disclosed herein orally (e.g., as a capsule, a tablet, a caplet, etc.), transdermally (e.g., as a gel or solution, a patch, an ointment, a cream, a lotion etc.), parenterally (by injection, intraductally, e.g., as a gel or solution), or in any other manner or form suitable for treatment and drug delivery. In some embodiments, the compositions disclosed herein are administered to a subject in need thereof as a monotherapy. In other embodiments, the compositions are administered as a combination therapy.

In one aspect, the methods comprise administering orally a pharmaceutical composition to a subject in need thereof. For example, the methods comprise administering to a subject any suitable oral dosage form (e.g., capsules, caplets, and tablets etc.) comprising a composition disclosed herein. As disclosed above, administering a composition comprising at least one therapeutic agent (such as a SERM, a SERD, an AI or a combination thereof), a fatty acid mixture comprising at least one omega-3 fatty acid (such as EPA and DHA etc.), and at least one vitamin D compound (such as cholecalciferol) is advantageous in treating ER+-related disorders and breast disorders, reducing the side effects of adjuvant therapy, and/or increasing treatment compliance.

In some embodiments, methods for treating a subject in risk for or having an estrogen-related disorder or a breast disorder comprise (i) placing in a nipple of the subject a breast duct device, and (ii) administering a therapeutically effect amount of a composition comprising at least one therapeutic agent, a fatty acid mixture comprising at least one omega-3 fatty acid, and at least one vitamin D compound to the subject. In other embodiments, methods for treating a subject in risk for or having an estrogen-related disorder or a breast disorder comprise (i) placing in a breast duct of the subject a breast duct device, and (ii) administering a therapeutically effect amount of a composition comprising at least one therapeutic agent, a fatty acid mixture comprising at least one omega-3 fatty acid, and at least one vitamin D compound to the subject. In some preferred embodiments, the method comprises an indwelling breast duct device. In other preferred embodiments, the method comprises retrieval of the breast duct device after administering a composition to a subject. In some embodiments, the administered compositions form a drug depot in the breast duct. Such methods comprise compositions capable of releasing active ingredients over time.

In some embodiments, methods of treatment comprises delivering a composition to a breast duct of a subject in need thereof, comprising placing in the breast duct a breast duct device comprising an indwelling unit being sized and configured for being positioned and maintained within a portion of a breast duct, the indwelling unit having an atraumatic distal end for positioning within the duct, and an elongated member extending from the unit, wherein the elongated member can be positioned to extend out of the breast duct when the indwelling unit is positioned within the breast duct, wherein the composition comprises at least one therapeutic agent, a fatty acid mixture comprising at least one omega-3 fatty acid, and at least one vitamin D compound.

In some embodiments, the method of treatment comprises delivering a composition to a breast duct of a subject in need thereof, comprising placing in the breast duct a breast duct device comprising an indwelling reservoir capable of dwelling in the breast duct, and a line or tube connected to the reservoir to reload the indwelling reservoir when empty, or to provide retrieval of the indwelling reservoir from the breast duct, wherein the composition released to the breast duct, wherein the composition comprises at least one therapeutic agent, a fatty acid mixture comprising at least one omega-3 fatty acid, and at least one vitamin D compound.

In some embodiments, the method of treatment comprises delivering a composition to a breast duct of a subject in need thereof, comprising placing in the breast duct a breast duct device comprising: a unit for holding a composition to be delivered to a breast duct, the unit being sized and configured to be positioned and supported on a nipple, and an elongated member for delivering the composition from the unit to the breast duct, the elongated member being in communication with the unit, being sized for positioning within the breast duct, and having a distal terminal end for positioning within the breast duct, said distal end having an atraumatic tip, wherein the composition comprises at least one therapeutic agent, a fatty acid mixture comprising at least one omega-3 fatty acid, and at least one vitamin D compound.

In some embodiments, the method comprises identifying and emulating a breast duct of a subject before placing the breast duct device in the breast duct of the subject. In other embodiments, the method comprises mapping a subject's breast duct before cannulating or placing the breast duct device in subject's breast duct. In some embodiments, the method comprises placing an indwelling breast duct device in a subject's breast duct.

In another aspect, the methods comprise administering a pharmaceutical composition to at least one breast by transdermal (i.e., topical) application. For example, the methods comprise applying a transdermal dosage form (device (e.g., a gel, a solution, a cream, an ointment, a lotion, etc.) or a drug delivery device e.g., a patch, a tape, bandage, etc.) directly to at least one breast or both breasts of a subject with or without an applicator, or spraying a composition onto the breast using an aerosolized or non-aerosolized spray. In another aspect, the methods comprise delivering a composition to a breast duct of a subject, comprising contacting the composition contained within a treatment chamber of a device with a nipple or an aureola of a breast and applying positive pressure on the composition.

In some embodiments, the method of treating a subject in risk for or having a breast disorder or an estrogen-related disorder comprising administering to the subject a pharmaceutical composition comprising: (i) a therapeutically effective amount of at least one therapeutic agent; (ii) 20% to 60% of a fatty acid mixture comprising at least one omega-3 fatty acid triglyceride selected from a group consisting of eicosapentaenoic acid, docosahexaenoic acid, alpha-linolenic acid, hexadecatrienoic acid, stearidonic acid, eicosatrienoic acid, eicosatetraenoic acid, eicosapentaenoic acid, heneicosapentaenoic acid, docosapentaenoic acid, clupanodonic acid, tetracosapentaenoic acid, tetracosahexaenoic acid, Nisinic acid, and a combination thereof; (iii) 10 IU to 6000 IU of at least one vitamin D compound; and (iv) a sufficient amount of fish oil.

In some embodiments, the method of treating a subject in risk for or having a breast disorder or an estrogen-related disorder comprising administering to the subject a pharmaceutical composition comprising: (i) a therapeutically effective amount of at least one therapeutic agent; (ii) 20% to 60% of a fatty acid mixture comprising at least one omega-3 fatty acid triglyceride selected from a group consisting of eicosapentaenoic acid, docosahexaenoic acid, alpha-linolenic acid, hexadecatrienoic acid, stearidonic acid, eicosatrienoic acid, eicosatetraenoic acid, eicosapentaenoic acid, heneicosapentaenoic acid, docosapentaenoic acid, clupanodonic acid, tetracosapentaenoic acid, tetracosahexaenoic acid, Nisinic acid, and a combination thereof; (iii) 10 IU to 6000 IU of at least one vitamin D compound; (iv) a sufficient amount of fish oil, and (v) 0.5% to 6% by weight of a gelling agent; and wherein the percentage are w/w of the gel. In some preferred embodiments, the at least one therapeutic agent is less than 5%.

Local delivery of the compositions via transdermal and intraductal administration in high concentrations without creating a high plasma concentration is a particular advantage of some of the embodiments disclosed herein.

Accordingly, methods comprise administration of doses of SERMs (tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923) that result in plasma concentration of less than 80 pg/mL, or the mean estradiol concentrations in normal premenopausal women. In preferred embodiments, doses of SERMs that result in plasma concentration of less than 50 pg/mL are preferred. Doses of SERDs, such as fulvestrant, that result in plasma concentration of less than 5 ng/mL are preferred. In a more preferred embodiment, doses of fulvestrant that result in plasma concentration of less than 2 ng/mL are preferred. Doses of AI such as anastrozole, letrozole, or exemestane that result in plasma concentration of less than 50 ng/mL are preferred. In a more preferred embodiment, doses of anastrozole letrozole, and exemestane that result in plasma concentration of less than 10 ng/mL are preferred. Advantages of this local administration and low plasma concentrations of a composition will be readily apparent to one of skill in the art.

In some embodiments, methods comprise administration to a subject in need thereof a daily dosage of the at least one therapeutic agent rangings in amounts from 0.1 mg/breast to 250 mg/breast, from 0.1 mg/breast to 200 mg/breast, from 0.1 mg/breast to 150 mg/breast, from 0.1 mg/breast to 100 mg/breast, 0.1 mg/breast to 50 mg/breast, from 0.5 mg/breast to 50 mg/breast, from 5 mg/breast to 45 mg/breast, from 5 mg/breast to 40 mg/breast, from 5 mg/breast to 35 mg/breast, from 5 mg/breast to 30 mg/breast, from 5 mg/breast to 25 mg/breast, from 5 mg/breast to 20 mg/breast, from 5 mg/breast to 15 mg/breast, from 5 mg/breast to 10 mg/breast, from 1 mg/breast to 25 mg/breast, from 2 mg/breast to 20 mg/breast, from 3 mg to 30 mg/breast, and from 4 mg/breast to 40 mg/breast. In some preferred embodiments, the composition may be administered transdermally or intraductally.

In preferred embodiments, a dose of the at least one therapeutic agent administered daily to the subject ranges from 0.1 mg/breast to 100 mg/breast. In more preferred embodiments, a dose of the at least one therapeutic agent administered daily to the subject is 0.1 mg/breast, 0.5 mg/breast, 1 mg/breast, 1.5 mg/breast, 2 mg/breast, 3 mg/breast, 4 mg/breast, 5 mg/breast, 10 mg/breast, 15 mg/breast, 20 mg/breast, or 25 mg/breast per day. Such a dose may be administered by transdermally and/or intraductally as described herein. In some embodiments, the at least one therapeutic dosed is a SERM, a SERD or an AI selected from the group consisting of tamoxifen, cis-tamoxifen, 4-hydroxytamoxifen, endoxifen, desmethyltamoxifen, lasofoxifene, raloxifene, arzoxifene, miproxifene, levormeloxifene, droloxifene, idoxifene, toremifene, EM652, ERA-923, fulvestrant, ARN-810, CH4986399, anastrozole, exemestane, and letrozole. The 4-hydroxytamoxifen may be administered transdermally to the subject at a dose of 0.25 mg/breast, 0.50 mg/breast, 0.75 mg/breast, 1 mg/breast, 2 mg/breast daily. A daily transdermal dose of 4-OHT of 0.50 mg/breast, 0.75 mg/breast, and 1 mg/breast are preferred. In other embodiments, fulvestrant administered transdermally to the subject is 0.25 mg/breast, 1 mg/breast, 5 mg/breast, 10 mg/breast, 20 mg/breast, 25 mg/breast, 30 mg/breast, 35 mg/breast and 40 mg/breast per day.

In some embodiments, methods comprise transdermally administering to a subject in need thereof a composition comprising: an amount of at least one therapeutic agent ranging from 0.1 mg/breast to 250 mg/breast, from 0.1 mg/breast to 200 mg/breast, from 0.1 mg/breast to 150 mg/breast, from 0.1 mg/breast to 100 mg/breast, 0.1 mg/breast to 50 mg/breast, from 0.5 mg/breast to 50 mg/breast, from 5 mg/breast to 45 mg/breast, from 5 mg/breast to 40 mg/breast, from 5 mg/breast to 35 mg/breast, from 5 mg/breast to 30 mg/breast, from 5 mg/breast to 25 mg/breast, from 5 mg/breast to 20 mg/breast, from 5 mg/breast to 15 mg/breast, from 5 mg/breast to 10 mg/breast, from 1 mg/breast to 25 mg/breast, from 2 mg/breast to 20 mg/breast, from 3 mg to 30 mg/breast, and from 4 mg/breast to 40 mg/breast; a therapeutically effective amount of the fatty acid mixture ranging from 50 mg/g to 150 mg/g EPA triglyceride or phospholipid and from 500 mg/g to 850 mg/g DHA triglyceride or phospholipid; and an amount of the at least one vitamin D compound ranging from 400 to 6000 IU, from 1000 to 4000 IU, from 2000 to 4000 IU, or 10 IU to 400 IU.

In other embodiments, methods comprise intraductal administration to a subject in need thereof an composition comprising at least one therapeutic agent ranging from 0.1 mg/g to 100 mg/g, from 0.5 mg/g to 45 mg/g, from 1 mg/g to 45 mg/g, from 1 mg/g to 40 mg/g, from 1 mg/g to 35 mg/g, from 1 mg/g to 30 mg/g, from 1 mg/g to 25 mg/g, from 1 mg/g to 20 mg/g, from 1 mg/g to 15 mg/g, from 1 mg/g to 10 mg/g, and from 1 mg/g to 5 mg/g by weight of gel per breast duct. In at least one preferred embodiment, the composition is administered per breast duct in amounts of 0.25 mg/g, 0.5 mg/g, 1 mg/g, 2 mg/g, 5 mg/g, 10 mg/g and 20 mg/g by weight of gel. In one aspect, the at least one therapeutic agent administered is tamoxifen, cis-tamoxifen, 4-hydroxytamoxifen, endoxifen, desmethyltamoxifen, lasofoxifene, raloxifene, arzoxifene, miproxifene, levormeloxifene, droloxifene, idoxifene, toremifene, EM652, ERA-923, fulvestrant, ARN-810, CH4986399, anastrozole, exemestane, letrozole, or a combination thereof. In at least one preferred embodiment, the at least one therapeutic agent administered is 4-OHT, tamoxifen, cis-tamixifen, desmotamoxifen, fulvestrant or anastrozole.

In another aspect, methods comprise a dose of at least one therapeutic agent administered to a subject ranging from 0.01 mg/mL to 45 mg/mL, from 0.5 mg/mL to 45 mg/mL, from 1 mg/mL to 45 mg/mL, from 1 mg/mL to 40 mg/mL, from 1 mg/mL to 35 mg/mL, from 1 mg/mL to 30 mg/mL, from 1 mg/mL to 25 mg/mL, from 1 mg/mL to 20 mg/mL, from 1 mg/mL to 15 mg/mL, from 1 mg/mL to 10 mg/mL, and from 1 mg/mL to 5 mg/mL per breast duct. In a preferred embodiment, the dose of the at least one therapeutic agent administered to the subject per breast duct is 20 mg/mL.

In one aspect, fulvestrant is administered to a subject in need thereof at a dose ranging from 1 mg/mL to 40 mg/mL.

In some embodiments, methods comprise administering to a subject a dose of a composition comprising: the at least one therapeutic agent in an amount ranging from 0.1 mg/mL to 45 mg/mL, from 0.5 mg/mL to 45 mg/mL, from 1 mg/mL to 45 mg/mL, from 1 mg/mL to 40 mg/mL, from 1 mg/mL to 35 mg/mL, from 1 mg/mL to 30 mg/mL, from 1 mg/mL to 25 mg/mL, from 1 mg/mL to 20 mg/mL, from 1 mg/mL to 15 mg/mL, from 1 mg/mL to 10 mg/mL, and from 1 mg/mL to 5 mg/mL; an amount of the fatty acid mixture ranging from 50 mg/g to 150 mg/g EPA triglyceride or phospholipid and from 550 mg/g to 850 mg/g DHA triglyceride or phospholipid; and an amount of the at least one vitamin D compound ranging from 10 IU to 400 IU, 400 to 6000 IU, from 1000 to 4000 IU, or from 2000 to 4000 IU per breast duct.

In some embodiments, methods comprise administering to a subject an oral dose of a composition comprising at least one therapeutic agent in amounts ranging from 0.1 mg to 50 mg, from 0.2 mg to 40 mg, from 0.3 mg to 30 mg, from 0.4 mg to 25 mg, from 0.5 mg to 20 mg, from 1 mg to 10 mg, from 0.1 mg to 10 mg, and from 0.5 mg to 5 mg per day. In a preferred embodiment, the oral dose ranges from 0.1 mg to 10 mg. In a more preferred embodiment, the oral dose is 0.5 mg or 1.0 mg. In at least one embodiment, the at least one therapeutic agent comprised in the composition being dosed orally is anastrozole.

In some embodiments, methods comprise administering to a subject a composition comprising a fatty acid mixture comprising EPA and DHA in amounts ranging from 50 mg/g to 150 mg/g EPA and from 650 mg/g to 750 mg/g DHA. The EPA may in the form of triglyceride or phospholipid. In other embodiments, the composition comprises the fatty acid mixture further comprising omega-3 fatty acids in amounts ranging from 750 mg/g to 900 mg/g omega-3 fatty acids. In yet other embodiments, a subject is administered a composition comprising a fatty acid mixture comprising at least 200 mg/g EPA, at least 500 mg/g DHA selected from phospholipid and triglyceride form. In still other embodiments, fatty acid mixture comprises at least 700 mg/g omega-3 fatty acids and 2000 IU-4000 IU of the at least one vitamin D compound.

In other embodiments, a subject is administered a composition comprising at least one vitamin D compound in an amount ranging from 25 μg to 200 μg per day. In other embodiments, a subject is administered a composition comprising at least one vitamin D compound in an amount ranging from 1 μg to 5 μg per day.

It will be appreciated by one of ordinary skill in the art that the treatment regimen for treating a breast disorder or an estrogen-related disorder with a composition comprising a SERM, a SERD, an AI, or a combination thereof, a fatty acid mixture comprising at least one omega-3 fatty acid, and at least one vitamin D compound may depend on a variety of factors, including the type, age, weight, sex, diet, and the medical condition of the subject, and pharmacological considerations such as activity, efficacy, pharmacokinetic and toxicology profiles of the particular API employed. Thus, the treatment regimen actually employed may vary widely from subject to subject. The present invention is also not limited to doses or methods of treatment and delivery described herein. One of skill in the art will recognize that the dosing regimens that are encompassed by the present invention include dosing the subject on a once a day, twice a day, thrice a day, weekly, biweekly, semimonthly, monthly, every 2 months, quarterly, every 6 month, and annual basis or any regimen a physician or healthcare professional may deem suitable.

The present invention further provides that the efficacy and safety of the compositions and the treatment regimen disclosed herein may be assessed during and after treatment. The present invention also provides that a subject's breast condition may be determined prior to prophylactic treatment with the compositions disclosed here. For example, the present invention provides that women with prior history and/or family history of breast disorders or estrogen-related disorders are tested for a risk of developing or for reoccurrence of breast disorders and/or estrogen-related disorders and administered with the compositions disclosed herein for prevention and treatment of such disorders. Accordingly, in some embodiments, the methods comprise assessing a subject's breast condition (such as risk for developing, recurrence, and prognosis of breast disorder and/or estrogen-related disorder) before, during and/or following treatment with the compositions disclosed herein. In some embodiments, methods comprise collecting biological samples from a subject, testing the biological samples, based on the test results predicting breast disorder and/or estrogen-related disorder status of the subject, and administering to a subject in need thereof a composition comprising at least one therapeutic agent, a fatty acid mixture comprising at least one omega-3 fatty acid, and at least one vitamin D compound.

Biological samples that may be collected from a subject includes nipple aspirate fluid (NAF), blood, plasma, serum, breast cells, or tissue, or a combination thereof. Methods of collecting NAF samples are known in the art (for example, U.S. Pat. No. 5,798,266, U.S. Pat. No. 6,689,073, U.S. Pat. No. 6,887,219). Preferably, these methods are non-invasive. The methods provided herein may be practiced with an appropriate breast pump device or ductal access tool which may be used for NAF sample collection, such as for example, a device described in U.S. Pat. No. 5,798,266; U.S. Pat. No. 6,689,073; U.S. Pat. No. 6,887,210; U.S. Pat. No. 6,413,228; and U.S. Pat. No. 6,689,070, each of which is incorporated herein by reference in its entirety. Such ductal access tools may have single elongated lumens for delivering fluids into the breast ducts and collecting NAF samples. NAF samples are useful for diagnosis and classification of breast disorders (U.S. Patent Application Pub. No. 2013/0115629, and U.S. Patent Application Pub. No. 2013/0130310, each of which is incorporated herein by reference in its entirety).

In one aspect, a method of treating a subject is provided wherein the method comprises: (a) collecting NAF sample from the subject: (b) testing NAF sample; (c) determining the subject's breast condition; (d) based on the breast condition, administering to the subject a composition comprising (i) at least one therapeutic agent; (ii) a fatty acid mixture comprising at least one omega-3 fatty acid; and (iii) at least one vitamin D compound. The test methods for assessing the effectiveness of a composition in the prevention and treatment of the breast disorders and estrogen-related disorders and/or the prognosis comprises measuring any number of genetic markers and other biomarkers in a subject over time, including, without limitation, presence of cells, cell free DNA (cfDNA), RNA, lipids, glycolipids, carbohydrates, proteins in nipple aspirate fluid (NAF) samples, changes in mean breast density, changes in biomarker expression (e.g., CK5, CK14, CK7, CK19, p53, uPA, PAI, and Gal-GalNac), etc. Other non-limiting examples of biomarkers useful for diagnostic tests (for example, for cell adhesion and/or cell motility markers such as Cathepsin D, plasminogen activators and collagenases) are disclosed in U.S. Pat. No. 6,610,484, U.S. Pat. No. 6,287,521, U.S. Pat. No. 6,689,073, U.S. Pat. No. 6,887,210, and U.S. Pat. No. 7,128,877, the disclosures of which are hereby incorporated in its entirety in the present application.

In some embodiments, methods comprising testing of NAF comprises collecting NAF sample from a subject, contacting a cell of the NAF sample adsorbed to device such as a slide or an adsorbent paper comprising antibodies or other reagents that bind to or otherwise identify biomarkers including, without limitation, cytokeratins such as CK5, CK14, CK7, CK18, Cyclin B1, MUC1, tumor suppressors such as p53, uPAR, PAI, and Gal-Gal-NAc. Binding of one or more antibodies to the cell is detected and the data is analyzed using various computer algorithms. For example, the presence of p63 would indicate the presence of basal-like breast cancer. In some embodiments, the presence of CK5/CK14 and CK7/CK18 would be indicative of usual ductal hyperplasia. In other embodiments, the presence of CK7/CK18, and reduced or negative presence of CK5/CK14 would be indicative of atypical ductal hyperplasia or DCIS. In other embodiments, increase in CK7/CK18 would indicate the presence of breast cancer, specifically luminal breast cancer. Invasive breast lesion would be indicated by a reduction in the number of or the absence of myopeithelial cells (CK1/CK14 and/or p63) and the presence of glandular epithelial cells (CK17/CK18). Primary breast carcinomas for example, would be indicated by an increase in luminal (duct-wall) cells and a decrease in the number of myoepithelial cells. In some embodiments, the absence or reduction in the number of myopeithelial cells and the presence of glandular cells would be indicative of invasive lesion.

Accordingly, in at least one embodiment, a method of treating a subject in need thereof comprises a test conducted on NAF sample comprising: a) collecting a NAF sample from a subject; b) contacting a cell of the NAF sample adsorbed to an adsorbent paper comprising antibodies that bind to CK5, CK14, CK7, CK18, and p63; c) detecting binding of one or more of the antibodies to said cell; and d) classifying cancer based upon the binding pattern of the antibodies. In some embodiments, the adsorbent paper comprises antibodies that bind to uPAI, PAR and Gal-GalNac.

Other diagnostic tests include measurement of total protein, evaluating the cytology (for e.g., cell shape, size, chromatin content, nucleus:cytoplasm ratio, etc.).

In other embodiments, the cells isolated from the NAF sample may be maintained or cultured in a laboratory to study its growth pattern and determine their propensity for uncontrolled proliferation. These cells may also be used for isolation and sequencing of subject's DNA (including mitochondrial DNA), RNA (including without limitation, micro-RNA, 16S RNA) and subjected to testing for the presence or absence of breast disorder biomarkers.

Breast disorder biomarkers are useful for classification of breast disorders, screening, diagnosing and monitoring progression of a breast disorder (or a lack thereof), and include, without limitation, identifying the presence or absence of gene mutations, single nucleotide polymorphisms (SNPs), gene or DNA copy number alterations or variations, alterations in DNA methylation patterns or signatures, histone methylation patterns or signatures, changes in micro-RNA patterns, 16S RNA sequences, altered micro-biome, altered expression of cancer biomarkers, for example, without limitation, BRCA1, BRCA2, etc. Various commercially available cancer gene panels (for example, BROCA test from University of Washington, Seattle, Wash., BRACAnalysis from Myriad Genetics, Inc. UT) are known in the art and are useful for the purpose of the present invention. In another aspect, a diagnostic test may comprise analysis of micro-RNA (MiRNA) in a patient sample, for example, the NAF. Recent studies on miRNA profiling have revealed differential expression of miRNAs in breast carcinomas compared to their normal tissue counterparts. For example, an increase in miR-155, miR-21, miR-27, miR10b expression would indicate the presence of breast cancer. In some embodiments, a decrease in miR-125 (a and b), miR145 and miR205 would be indicative of breast cancer. In some embodiments, an increase in miR-155, miR-21, miR-27, miR10b and a decrease in miR-125 (a and b), miR145 and miR205 would be indicative of breast cancer. In other embodiments, decrease in miR-140, a tumor suppressor, would be indicative of DCIS and/or IDC and suggest increased breast cancer progression.

In one aspect, the methods of screening, diagnosing, and monitoring for the presence or absence of gene mutations include, without limitation, sequencing of individual genes or a panel of genes or gene clusters by dideoxy, Sanger sequencing, next generation sequencing, single cell sequencing, single nucleus sequencing, single molecule real time sequencing, whole genome sequencing, exome sequencing, RNA sequencing from single cells, microarray analysis, PCR, etc. Based on the results of the tests, a subject's responsiveness to treatment, prognosis, or likelihood of developing such disorders or recurrence may be determined or predicted. The dose and/or the treatment regimen may be then be established and/or adjusted based on the test results.

In some embodiments, methods comprise a test conducted on NAF sample is selected from a group consisting of gene mutations, single nucleotide polymorphisms (SNPs), copy number, DNA methylation patterns or signatures, histone methylation patterns or signatures, micro-RNA patterns, micro-biome pattern, and cancer biomarkers.

In other embodiments, methods comprise a test conducted on NAF sample comprising: a) collecting a NAF sample from a subject; b) conducting a single nucleus sequencing of a cell of the NAF sample; c) determining the risk for, presence, or reoccurrence of a cancer; and d) administering a therapeutically effective amount of a composition to the subject. Methods for conducting a single cell sequencing are known in the art (Wang et al. Nature. 2014, 512:155-160). Such a single nucleus or single cell sequencing may be conducted on a whole genome or exome basis. Such single nucleus sequencing is advantageous for identifying cell lineage of the breast cancer, particularly under conditions when number of cells in samples is very low, for example, in NAF samples. In some embodiments, methods disclosed herein require less than 100 cells, less than 50 cells, less than 10 cells, less than 9 cells, less than 8 cells, less than 7 cells, less than 6 cells, less than 5 cells, less than 4 cells, less than 3 cells, less than 2 cells. More preferably, these methods require 2 cells. Even more preferably, these methods require a single cell. Methods requiring a single cell are particularly advantageous in performing diagnostic tests using techniques such as single nucleus sequencing on samples from subject using non-invasive methods.

In at least one embodiment, the method of treatment comprises a) collecting a NAF sample from a subject; b) providing at least one cell from the NAF sample; c) conducting a whole-genome sequencing; d) determining the subject's risk for, presence or reoccurrence of breast disorder or estrogen-related disorder; and e) administering a therapeutically effective amount of a pharmaceutical composition, wherein the composition comprises at least one therapeutic agent, a fatty acid mixture comprising at least one omega-3 fatty acid, and at least one vitamin D compound.

In some embodiments, the method for treatment comprises administering to the subject a pharmaceutical composition comprising: at least one therapeutic agent; a fatty acid mixture comprising at least one omega-3 fatty acid; and at least one vitamin D compound. In some embodiments, the at least one therapeutic agent is a SERM, a SERD, an AI, or a combination thereof, and pharmaceutically acceptable salts thereof. In some embodiments, the SERM is selected from the group consisting of tamoxifen, cis-tamoxifen, 4-OHT, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923. In some embodiments, the SERM is 4-OHT, desmethyltamoxifen, or endoxifen. In some embodiments, the SERD is a fulvestrant, ARN-810, or CH4986399. In some embodiments, the SERD is fulvestrant. In some embodiments, the AI is selected from the group consisting of anastrozole, exemestane and letrozole. In some embodiments, the AI is anastrozole. In some embodiments, the at least one therapeutic agent is between 0.01% to 15% by weight of the composition. In some embodiments, the at least one omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the omega-3 fatty acid is a triglyceride or a phospholipid. In some embodiments, the fatty acid mixture comprising at least one omega-3 fatty acid is between 10% to 90% by weight of the composition. In some embodiments, the fatty acid mixture comprises from 400 mg/g to 600 mg/g of the at least one omega-3 fatty acid. In some embodiments, the fatty acid mixture comprises a plurality of omega-3 fatty acids. In some embodiments, the fatty mixture comprises a mixture of EPA and DHA. In some embodiments, the fatty acid mixture is a fatty acid oil mixture. In some embodiments, the fatty acid oil mixture is derived from at least one oil selected from a group consisting of a marine oil, a plant-based oil, an algae oil, and a microbial oil. In some embodiments, wherein the marine oil is a fish oil. In some embodiments, the fatty acid mixture is an emulsion. In some embodiments, the emulsion is an alcohol-in oil emulsion, an oil-in-alcohol emulsion, oil-in-water emulsion, water-in-oil emulsion, water-in-oil-in-water emulsion, or an oil/alcohol/water emulsion. In some embodiments, the at least one vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4,1,25 dihydroxy-19-nor-vitamin D2, 1-alphahydroxyvitamin D3, vitamin D analogs, and a combination thereof. In some embodiments, the at least one vitamin D compound is cholecalciferol. In some embodiments, the at least one vitamin D compound is partly or wholly dissolved, dispersed, or suspended in the fatty acid mixture comprising the at least one omega-3 fatty acid. In some embodiments, the at least one therapeutic agent and the at least one vitamin D compound are partly or wholly dissolved, dispersed or suspended in the fatty acid mixture comprising the at least one omega-3 fatty acid. In some embodiments, the at least one vitamin D compound has an activity ranging between 10 IU-6000 IU. In some embodiments, the composition further comprises an excipient. In some embodiments, the composition is formulated in a gel, a solution, a lotion, an ointment, a cream, or an emulsion. In some embodiments, the gel comprises a vehicle, co-solvent, a stabilizing agent, a neutralization agent, a permeation enhancer, an absorption enhancer, a surfactant, a gelling agent, a polymer, a co-polymer, a cross-linking agent, an antioxidant, a moisturizer, an antimicrobial, a preservative, or a combination thereof. In some embodiments, the vehicle is an oily vehicle. In some embodiments, the oily vehicle is fish oil. In some embodiments, the gelling agent is HPMC, CMC, Carbopol or polyacrylic acid. In some embodiments, the permeation enhancer is an ether, a sulfoxide, a poloxomer, a pyrrolidone, an azone, or a fatty alcohol. In some embodiments, the surfactant is SDS, cetrimide, Capmul, Cremaphor, or Tween 85. In some embodiments, the antioxidant is alpha-tocopherol, BHA, BHT, ascorbic acid and pharmaceutically acceptable salts and esters thereof, propyl gallate, citric acid and pharmaceutically acceptable salts thereof, malic acid and pharmaceutically acceptable slats thereof, and sulfite salts and mixtures thereof.

In some embodiments, the method further comprises at least one additional medication. In some embodiments, the at least one additional medication is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof. In some embodiments, the at least one additional medication is trastuzumab.

In some embodiments, the method of treatment comprises administering to the subject a pharmaceutical composition comprising: 0.01 g to 15 g of at least one therapeutic agent; 1 g to 10 g of the fatty acid mixture comprising at least one omega-3 fatty acid; 10 IU to 6000 IU of the at least one vitamin D compound; and a fish oil (qs) to 100 g; wherein the composition is capable of being delivered locally to a tissue.

In some embodiments, the method of treatment comprises administering to the subject a pharmaceutical composition comprising: 0.01% to 15% of a SERM, a SERD, an AI or a combination thereof, or pharmaceutically acceptable salts thereof; 10% to 90% of a fatty acid mixture comprising at least one omega-3 fatty acid; 10 IU to 6000 IU of at least one vitamin D compound or pharmaceutically acceptable salts thereof; and 10% to 90% vehicle. In some embodiments, the composition is capable of being delivered locally to a tissue.

In some embodiments, the method of treatment further comprises at least one gelling agent. In some embodiments, the at least one gelling agent is 0.1% to 80% w/w of the composition.

In some embodiments, the SERM is selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof. In some embodiments, the SERD is selected from a group consisting of fulvestrant, ARN-810, and CH4986399 and pharmaceutically acceptable salts thereof. In some embodiments, the AI selected from a group consisting of anastrozole, exemestane, and letrozole, and pharmaceutically acceptable salts thereof. In some embodiments, the composition is delivered using a transdermal, a transpapillary, or an intraductal device. In some embodiments, the transdermal device is selected from the group consisting of an applicator, a patch, a tape, sheet, a dressing, a spray device and an aerosolizer. In some embodiments, the patch for transdermal delivery comprises: a backing layer; and an adhesive layer having a skin-contacting adhesive surface; wherein the adhesive layer comprises a drug reservoir comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

In some embodiments, the patch for transdermal delivery comprises: a backing layer; a drug reservoir disposed on a first layer; and a skin-contacting second layer comprising a pressure sensitive adhesive layer; wherein the second layer is attached to a surface of the first layer opposed to a surface in contact with the backing layer, wherein the second layer is a rate-controlling layer and wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days. In some embodiments, the patch for transdermal delivery comprises: a backing layer; a drug reservoir disposed on a first layer; a second layer comprising a rate-controlling membrane, the membrane being attached to a surface of the first layer opposed to a surface in contact with the backing layer; and a skin-contacting third layer comprising a pressure sensitive adhesive attached to a surface of the membrane that is opposed to the surface of the rate-controlling membrane in contact with the first layer; wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

In some embodiments, the method comprises: delivering the composition to a breast duct of the subject, comprising contacting the composition contained within a treatment chamber of a device with a nipple of a breast; and applying positive pressure on the composition. In some embodiments, the method comprises delivering the composition to a breast duct of a subject using a device selected from the group consisting of syringe and needle, microneedles, catheters, microcatheters, beads, and microbeads. In some embodiments, the method comprises delivering the composition to a breast duct of a subject, comprising placing in the breast duct a breast duct device comprising an indwelling reservoir capable of dwelling in the breast duct, and optionally, a line or tube connected to the reservoir to reload the indwelling reservoir when empty, or to provide retrieval of the indwelling reservoir from the breast duct, wherein the composition released to the breast duct.

In some embodiments, the method for treatment comprises administering to the subject an oral pharmaceutical composition comprising: at least one therapeutic agent; a fatty acid mixture comprising at least one omega-3 fatty acid; and at least one vitamin D compound. In some embodiments, the at least one therapeutic agent is tamoxifen, cis-tamoxifen, endoxifen, 4-OHT, desmethyltamoxifen, 4-hydroxy-N-desmethyl tamoxifen, lasofoxifene, raloxifene, benzothiphene, bazedofoxifene, arzoxifene, miproxifene, levormeloxifene, droloxifene, clomifene, idoxifene, toremifene, EM652, ERA-923, fulvestrant, ARN-810, CH4986399, anastrozole, exemestrane, and letrozole. In some embodiments, the composition is formulated in a capsule, a caplet, and a tablet. In some embodiments, the capsule is a gelatin capsule, gelatin-free capsule, a cap-in-cap capsule, alginate capsule, an HPMC capsule, a PVA capsule, and a seamless capsule. In some embodiments, the capsule is a hard capsule or a soft capsule. In some embodiments, the capsule comprises: a shell comprising 0.1 mg to 500 mg of a SERM, a SERD, an AI, or a combination thereof; and a fill phase comprising (a) 20% to 60% of a fatty acid mixture comprising at least one omega-3 fatty acid triglyceride or phospholipid; and (b) 10 IU to 6000 IU of at least one vitamin D compound. In some embodiments, the soft capsule comprises: a shell comprising 0.5 mg or 1 mg of anastrozole; a fill phase comprising (a) 60% of a fatty acid mixture comprising at least 99% EPA triglyceride or phospholipid; and (b) 400 IU of cholecalciferol; and a sufficient amount of fish oil.

In some embodiments, the composition further comprises at least one additional medication. In some embodiments, the at least one additional medication is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof. In some embodiments, the at least one additional medication is trastuzumab.

In some embodiments, the method comprises delivering to a subject a daily dose of the at least one therapeutic agent ranging from 0.1 mg/breast to 250 mg/breast, from 0.1 mg/breast to 200 mg/breast, from 0.1 mg/breast to 150 mg/breast, from 0.1 mg/breast to 100 mg/breast, 0.1 mg/breast to 50 mg/breast, from 0.5 mg/breast to 50 mg/breast, from 5 mg/breast to 45 mg/breast, from 5 mg/breast to 40 mg/breast, from 5 mg/breast to 35 mg/breast, from 5 mg/breast to 30 mg/breast, from 5 mg/breast to 25 mg/breast, from 5 mg/breast to 20 mg/breast, from 5 mg/breast to 15 mg/breast, from 5 mg/breast to 10 mg/breast, from 1 mg/breast to 25 mg/breast, from 2 mg/breast to 20 mg/breast, from 3 mg to 30 mg/breast, and from 4 mg/breast to 40 mg/breast. In some embodiments, the subject is administered a daily dose of the endoxifen of 0.25 mg/breast, 0.75 mg/breast, 1 mg/breast, or 2 mg/breast. In some embodiments, the subject is administered a daily dose of fulvestrant administered of 0.25 mg/breast, 1 mg/breast, 5 mg/breast, 10 mg/breast, 20 mg/breast, 25 mg/breast, 30 mg/breast, 35 mg/breast or 40 mg/breast. In some embodiments, the dose of the at least one therapeutic agent administered to the subject per breast duct is 0.25 mg/g, 0.5 mg/g, 1 mg/g, 2 mg/g, 5 mg/g, 10 mg/g and 20 mg/g by weight of gel. In some embodiments, an intraductal administration of the composition to a subject delivers the at least one therapeutic agent at a dose of 20 mg/mL per breast duct. In some embodiments, the composition is administered intraductally in a volume ranging from 0.1 mL to 2 mL, 0.1 mL to 1.5 mL, and 0.5 mL to 1 mL. In some embodiments, a blood or plasma concentration of the at least one therapeutic agent is less than 50 ng/ml for at least 3 days.

In some embodiments, methods for treatment comprise: collecting a NAF sample from the subject: testing the NAF sample using a testing method; determining subject's breast condition; based on subject's breast condition, administering to the subject an amount of a pharmaceutical composition comprising (a) at least one therapeutic agent; (b) a fatty acid mixture comprising at least one omega-3 fatty acid; and (c) at least one vitamin D compound. In some embodiments, the testing is selected from a group consisting of determining presence or absence of gene mutations, single nucleotide polymorphisms variations, gene copy number variations, DNA copy number variations, alterations in DNA methylation patterns, changes in histone methylation patterns, changes in micro-RNA patterns, altered micro-biome, altered cytology, and altered expression of cancer biomarkers. In some embodiments, testing comprises conducting: (a) cytology tests; (b) a single nucleus sequencing; (c) single cell sequencing; (d) microarray analysis; (e) PCR analysis; (f) immunohistochemistry; (g) immunofluorescence; (h) and 16S RNA sequencing; and (i) RFLP. In some embodiments, the sequencing comprises dideoxy sequencing, Sanger sequencing, next generation sequencing, single molecule real time sequencing, whole genome sequencing, exome sequencing, and RNA sequencing. In some embodiments, the testing comprises: collecting a NAF sample from the subject; contacting a cell of the NAF sample adsorbed to an adsorbent paper comprising antibodies that bind to CK5, CK14, CK7, CK18, and p63; detecting binding of one or more of the antibodies to said cell; and classifying the breast condition based upon the binding pattern of the antibodies.

In some embodiments, methods for treatment comprise: collecting a NAF sample from the subject; providing at least one cell from the NAF sample; conducting a whole-genome sequencing of the cell; determining the subject's risk for, presence or reoccurrence of breast disorder or estrogen-related disorder; and administering a therapeutically effective amount of a pharmaceutical composition; wherein the composition comprises (a) at least one therapeutic agent, (b) a fatty acid mixture comprising at least one omega-3 fatty acid, and (c) at least one vitamin D compound.

### VI. Combination Therapies

In some embodiments, the compositions are administered to subjects in combination with other therapies. In some other embodiments, the compositions are used in combination with at least one additional medication. Where combination therapy is administered, the at least one additional medication may be included in a single composition as described herein or be administered separately (e.g., simultaneously or sequentially). Therapeutically effective amounts of the at least one additional medication are well known to those skilled in the art. However, it is well within the attending physician or healthcare professional's ability to determine the amount of the at least one additional medication to be delivered when administered as an adjuvant therapy, simultaneously or sequentially.

In one aspect, the at least one additional medication is dissolved, dispersed, or suspended in the single composition comprising the at least one therapeutic agent, the fatty acid comprising the at least one omega-3 fatty acid, and the at least one vitamin D compound.

In yet another aspect, the at least one additional medication is dissolved, dispersed or suspended in a fatty acid fatty acid mixture comprising the at least one omega-3 fatty acid, and the at least one vitamin D analog and a pharmaceutically acceptable carrier thereof.

When the at least one additional medication is in an oral dosage form (such as a capsule, caplet or a table), it may be comprised in the shell or it may be comprised in the fill phase or both of such oral dosage form.

A. Anti-Cancer Agents

In some embodiments, the at least one additional medication is a chemotherapeutic. Examples of chemotherapeutics include those recited in the “Physician's Desk Reference”, 64th Edition, Thomson Reuters, 2010, which is hereby incorporated by reference. Methods for the safe and effective administration of most of these chemotherapeutic agents are known to those skilled in the art. In addition, their administration is described in the standard literature. For example, the administration of many chemotherapeutic agents is described in “Physician's Desk Reference” (PDR, e.g., 2010 edition, PDR Network, Montvale, N.J.), the disclosure of which is also incorporated by reference in its entirety.

In brief, non-limiting examples of chemotherapeutics include alkylating agents, antineoplastic agents, anti-mimetics, anti-metabolites, anti-tumor antibiotics, topo-isomerase inhibitors, mitotic inhibitors, corticosteroids, differentiating agents, anti-cancer antibodies, anti-cancer target drugs, immunotherapy agents, anthracyclins, platinums, vinca alkaloids, camptothecins, hormones, miRNA etc.

In one aspect, the chemotherapeutic is doxorubicin, paclitaxel or derivative thereof, 5-FU, and carboplatin or a derivative thereof.

Suitable antineoplastic agents that can be dosed in combination with the compositions described herein include, for example without limitation, alkylating agents (including without limitation, nitrogen mustards, ethylenimine derivatives, alkyl sulfonates, nitrosoureas, and triazine), uracil mustard, cyclophosphamide (Cytoxan™), chlormethine, ifosfamide, melphala, chlorambucil, pipobroman, triethylene melamine, triethylenethiophosphoramine, busulfan, carmustine, lomustine, streptozocin, dacarbazine, temozoloide, and combinations thereof.

Other chemotherapeutic or anti-cancer agents include, for example without limitation, antimetabolites (including without limitation, folic acid antagonists or antifolates, pyrimidine analogs, purine analogs, and adenosine deaminase inhibitors) such as methotrexate, fluorouracil, gemcitabine, and combinations thereof. Suitable chemotherapeutic or anti-cancer agents further include certain natural products and their derivatives, for example without limitation, vinca alkaloids, anti-tumor antibiotics, enzymes, lymphokines, and epipodohyllotoxins) such as vinblastine, doxorubicin, vincristine, vindesine, bleomycin, dactinomycin, daunorubicin, epirubicin, idarubicin, ara-C, paclitaxel (TAXOL™), deoxycoformycin, mitomycin-C, mithramycin, L-asparagine, interferons (particularly IFN-a), etoposide, and teniposide and combinations thereof.

The present invention also contemplates that compositions may be used in combination with other anti-cancer agents such as antibody therapeutics or anticancer antibodies. In a further embodiment, the additional medication is a targeted anti-cancer antibody, i.e., an antibody which targets a specific tumor type. The term “antibody” is used in the broadest sense and specifically covers intact monoclonal antibodies, polyclonal antibodies, chimeric antibodies, humanized antibodies, multispecific antibodies (e.g. bispecific antibodies) formed from at least two intact antibodies, and antibody fragments so long as they exhibit the desired biological activity. The term “Antibody fragments” comprises a portion of an intact antibody, preferably the antigen binding or variable region of the intact antibody. Examples of antibody fragments include Fab, Fab′, F(ab′)2, and Fv fragments; diabodies; linear antibodies (Zapata et al. Protein Eng. 8(10):1057-1062, 1995); single-chain antibody molecules; and multispecific antibodies formed from antibody fragments. The targeted anti-cancer antibody may be a monoclonal or polyclonal antibody and may be selected from those described in Pasquetto et al., “Targeted Drug Delivery Using Immunoconjugates: Principles and Applications”, J. Immunother., 34(9):611-628 (November-December 2011), which is hereby incorporated by reference.

In one aspect, the targeted anti-cancer antibody is one or more of gemtuzumab (Mylotarg), alemtuzmab (CAMPATH™), rituximab (Rituxin, Mabthera), trastuzumab (Herceptin™), nimotuxumab, cetuximab (Erbitux), erlotinib (TARCEVA®, Genentech/OSI Pharm.), bevacizumab (Avastin™), pertuzumab (OMNITARG®, rhuMab 2C4, Genentech), Brentuximab vedotin (Adcetris™), Ipilimumab (MDX-101 and also known as Yervoy), Ofatumumab (Arzerra), Panitumumab (Vectibix), and Tositumomab (Bexxar), among others. In another aspect, the targeted antibody is one or more of alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab. In another embodiment, the at least one additional medication includes antibodies to immune co-stimulatory molecules including but not limited to CTLA-4, 4-1BB and PD-1, antibodies to cytokines (including but not limited to IL-10, TGF-beta, etc.), and chemokine receptors including but not limited to CCR2, CCR4 etc., among others

In other embodiments, the at least one additional medication is a targeted drug. The term “targeted drug” as used herein refers to a therapeutic agent that blocks cancer cell growth by interfering specific “targeted” molecules which are required for tumor growth. See, Pasquetto above, which is hereby incorporated by reference. In one aspect, the targeted drug includes, without limitation, dasatnib, imatinib, nilotinib, bosutnib, lestaurtinib, ruxolitinib, crizotinib, vandetabib, cabozantinib, afibercept, adipotide, denileukin diftitox, everolimus, and temosirolimus, among others.

Other chemotherapeutic or anti-cancer agents include, for example, cytotoxic agents such as platinum coordination agents (for e.g., cisplastin, and carboplatin), antineoplastic enzymes, topoisomerase inhibitors, biological response modifiers, growth inhibitors, hematopoetic growth factors, immune modulators, chemokines, cytokines (for example a granulocyte-macrophage colony stimulating factor (GM-CSF) or FLT3-ligand), cell migration blockers, and inhibitors of angiogenesis. Angiogenesis inhibitors include, but are not limited to, angiostatin, endostatin, thrombospondin, platelet factor 4, Cartilage-derived inhibitor (CDI), retinoids, Interleukin-12, tissue inhibitor of metalloproteinase 1, 2 and 3 (TIMP-1, TIMP-2, and T1MP-3) and proteins that block the angiogenesis signaling cascade, such as anti-VEGF (Vascular Endothelial Growth Factor) and IFN-alpha.

Alternatively, the compositions may be used to augment the effects of radiation therapy, which may be delivered locally to the tumor or to the whole body. In yet another embodiment, the at least one additional medication is hormonal therapy. The term “hormonal therapy” as used herein refers to a medication that blocks cancer cell growth by interfering with the activity of specific hormones such as testosterone, or dihydrotestosterone.

Accordingly, the at least one additional medication is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, hormones, 24-hydroxylase inhibitors, or a combination thereof. In one embodiment, the at least one additional medication is an anticancer antibody. In a preferred embodiment, the at least one additional medication is tratuzumab (Herceptin™)

B. Vitamin D Compound Catabolism Inhibitors

In another aspect, the at least one additional medication may comprise inhibitors of vitamin D catabolism, for example, inhibitors of the enzyme 24-hydroxylase. 24-hydroxylase reduces the levels of circulating levels of active forms of vitamin D to less active forms that excreted primarily by feces. Non-limiting examples of such inhibitors include soy isoflavone and genistein.

C. MiRNA

In another aspect, the at least one additional medication administered to a subject in combination with the compositions disclosed herein may comprise micro-RNA (MiRNA), upregulators or downregulators of miRNA, or a combination thereof. Recent studies on miRNA profiling have revealed differential expression of miRNAs in breast carcinomas compared to their normal tissue counterparts. For example, miR-155, miR-21, miR-27, miR10b, are upregulated and oncogenic in nature while miR-125 (a and b), miR145 and miR205 were downregulated. Other studies have shown that miR-140 serves as a tumor suppressor in both DCIS and IDC through interactions with SOX2 and SOX9, and loss of MiR-140 expression results in increased breast cancer progression. As non-limiting examples, the compositions of the present invention may be administered to a subject in combination with miR-125a, miR-125b, miR200, miR145, miR205, mi146a, let-7a-d, miR-26a, miR34, miR31miR-101, miR200b, miR-335, miR-126, miR-206, miR-17-5p and miR-140 or upregulators thereof. Other non-limiting examples, the compositions of the present invention may be administered to a subject in combination with downregulators of miR-155, miR10b, miR21, miR27 and miR-520c and miR-373.

D. DNA Methylation Modulators

In another aspect, the at least one additional medication administered to a subject in combination with the compositions disclosed herein may comprise, DNA methylation modulators. Aberrations in DNA methylation and in the proteins involved in DNA methylation are known to occur in cancer; for example, hypermethylation of tumor suppressor genes (Radpour et al., PLoS ONE, January 2011, 6:1 e16080), aberrant expression of DNA methyltransferase-1 (DNMT1) and other DNMTs (Baylin et al., Hum. Mol. Genet. 2001, 10:687-692), and hypomethylation of unique genes and repetitive sequences (Soares et al., Cancer. 1999 Jan. 1; 85(1):112-8; Ehrlich M., Oncogene 2002, 21:5400-5413). Accordingly, the present invention encompasses compositions and treatment methods that comprise administering DNA methylation modulators in combination with the compositions of the present invention. In some embodiments, the DNA methylation modulator is a DNA methylation inhibitor. Examples of DNA methylation inhibitors include without limitation, 5-Azacytidine, 5-aza-2′-deoxycytidine, and MG98. In a preferred embodiment, at least one additional medication is selected from a group consisting of 5-Azacytidine, 5-aza-2′-deoxycytidine, and MG98.

In some embodiments, the DNA methylation modulator is a DNA methylation activator. In a preferred embodiment, the DNA methylation activator is S-Adenosylmethionine (SAM) (Luo et al., Human Gastric Cancer and Colon Cancer. Int. J. Biol. Sci. 2010; 6(7):784-795). DNA hypomethylation in breast carcinoma is correlated with prognostic factors and tumor progression.

In one aspect, the subject's DNA methylation profile or signature is determined from cancer samples such as breast tissue sample, NAF and ductal fluid samples and blood or serum samples, prior to treatment by methods known in the art, such as bisulfite mapping, methylation sensitive PCR or methylation-sensitive restriction analyses of specific cancer genes. Several methylated genes and cancer genes are known in the art, including, without limitation, BRCA1, BRCA2, APC, BIN1, CST6, GSTP1, P16, P21, ESR-b, CIMP, and TIMP3. In a preferred embodiment, the subject's DNA methylation profile or signature is determined on a sample selected from a group consisting of breast tissue, NAF and ductal fluid and blood samples.

In another aspect, a subject's DNA methylation signature may be determined using whole genome approaches such as single nucleus sequencing, multiplex sequencing, next generation sequencing (NGS) of bi-sulfite covered DNA (Lister et al., Nature 2009, 462:315-322), methylated DNA immunoprecipitation (MeDIP) followed by either hybridization to high density oligonucleotide arrays (Keshet et al., Nat Genet. 2006, 38:149-153) or NGS (Ruike, Y et al., BMC Genomics 2010, 11:137), and dedicated Illumina 27K and 450K arrays that measure the state of methylation of well-characterized CG sites distributed in the genome (Bibikova et al., Genomics 2011, 98:288-295). In some embodiments, a subject's methylated DNA may be enriched prior to DNA methylation mapping.

E. Other Combinations

In another aspect, the compositions described herein may be combined with radiotherapy, probiotic bacteria, natural substances and neutraceuticals (e.g., green tea epigallocatechin gallate (EGCG) and reservatrol), hormone therapy (e.g., Selective Androgen Receptor Modulators (SARMs) such as enobosam (ostarine, MK-2866, GTx-024), BMS-564,929, LGD-4033, AC-262,356, JNJ-28330835, LGD-3303, S-40503 and S-23), anti-inflammatory agents (such as COX-2 inhibitors and non-steroidal anti-inflammatory drugs (NSAIDs) such as Celexicob (Celebrex), Vioxx, Meloxicam, ibuprofen, naproxen (Anaprox, Naprosyn), diclofenac (Cambia, Cataflam, Voltaren), etodolac (Lodine), fenoprofen (Nalfon), flurbiprofen (Ansaid) and oxaprozin (Daypro)), cholesterol-lowering drugs such as statins (e.g., atorvastatin, cerivatstatin, fluvastatin, lovastatin, mevastatin, pitavastatin, pravastatin, rosuvastatin, simvastatin etc.), Poly(ADP-ribose) polymerase (PARP) inhibitors such as iniparib (BSI 201), BMN-673, Olaparib (AZD-2281), Rucaparib (AG014699, PF-01367338), Veliparib (ABT-888), MK 4827, BGB-290 and 3-aminobenzamide, inhibitors of mammalian target of rapamycin (mTOR), PI3K and IGF1R, and retinoids.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

### Composition of a 4-Hydroxytamoxffen Gel Formulation

This example provides the composition of a gel-formulated preparation according to the present disclosure and as shown below in Table 2.

TABLE 2

| | Quantity per | |
|---|---|---|
| Ingredient | 100 g of gel | |
| Endoxifen | 0.015 g and 0.03 g | |
| Fatty acid mixture-EPA triglyceride | 3.0 | g |
| Cholecalciferol | .001 | g |
| Isopropyl palmitate, US USP | 1 | g |
| HPMC | 1.5 | g |
| Fish Oil | q.s. 100 | g |

### Example 2

### Composition of Fulvestrant Gel Formulation

This example provides the composition of a gel-formulated preparation according to the present disclosure and as shown below in Table 3.

TABLE 3

| | Quantity per | |
|---|---|---|
| Ingredient | 100 g of gel | |
| Fulvestrant | 0.02 g and 0.04 g | |
| Fatty acid mixture-EPA:DHA triglyceride (1:1.2) | 4.0 | g |
| Cholecalciferol | .001 | g |
| Ethanol:Isopropanol (1:1 v/v) US USP | 60 | g |
| CMC | 5 | g |
| Cod fish oil | q.s. 100 | g |

### Example 3

### Preparation of an Anastrozole Soft Gelatin Capsule

This example describes the preparation of Anastrazole soft gelatin capsules and analyses thereof according to an aspect of the present disclosure.

### Preparation of a Soft Gelatin Capsule Shell

A gel mass is prepared as follows: Acid-insoluble polymer, Eudragit R L30D 55 will be dissolved in water-alkali vehicle. Triethyl citrate will be added. The film-forming agent, gelatin (lime bloom, 150 Bloom) will be mixed with plasticizer sorbitol and added to enteric polymer solution, mixed for 2 hours and kept overnight at 60° C. Table 4 provides an exemplar composition.

TABLE 4

| Ingredient | Percentage (%) |
|---|---|
| Gelatin | 27 |
| Eudragit L30D 55 | 20 |
| Sorbitol | 15 |
| Triethyl citrate | 2 |
| Ammonium Hydroxide | 5 |
| Water | 31 |

Gel mass will be cast as a ribbon with 0.03″ thickness on a cold drum (10° C. to 13° C.). Two sets of ribbons will be prepared and a rotary die process is used to cut the ribbons to an appropriate size and shape. The two ribbon pieces are melded together under heat to create a seamed soft gelatin capsule shell.

Next, Anastrozole will be solubilized in ethanol/isopropanol mixture (1:1 v/v). A film forming agents will be prepared by mixing, while stirring, a sufficient amount of a polymer, HPMC, with anastrozole solution at a ratio of anastrozole to HPMC of 45:55. Antifoam A (3 drops) will be added to the mixture. The film-forming agent/anastrozole mixture is sprayed onto an outer surface of capsule shell in amounts sufficient to deliver 0.5 mg of anastrozole/capsule to a subject. Weights of the shell (solid phase) and liquid fill phase will be approximately 150 mg/capsule and 1000 mg/capsule, respectively. The capsule shell is used to encapsulate the fill phase formulation described below.

### Methylene Blue Permeation Studies Using a Dialysis Chamber

The permeability of soft gelatin capsule shells will be tested to determine potential leakage of a fill phase formulation from the capsule shell. A dialysis set with two chambers is set up with a capsule shell gel mass ribbon acting as a separator for two chambers. A water soluble dye, methylene blue dissolved in 0.1 N HCl is placed in one chamber. In the second chamber separated by the capsule shell gel mass will be placed 0.1 N HCl without the dye. The dialysis chamber setup will be placed in shaking water bath at 37° C. Samples are removed at periodic time intervals of 10, 20, 30, 45, 60, 90, 120 and 180 minutes. It is expected that up to 60 minutes, no significant release of methylene blue dye will be observed. Any release after 60 min is expected to be less than 10%.

### Preparation of a Fill Phase Formulation (Omega-3 Oil and Cholecaliferol in Liquid Phase)

A fill phase formulation is prepared that comprises a fatty acid mixture comprising omega-3 fatty acids, EPA and DHA in triglyceride forms. An alcoholic mixture is prepared by adding ethanol to isopropanol (1:1 v/v) and stirred. A fatty acid mixture comprising EPA triglyceride and DHA triglyceride in 1.2:1 ratio, is hearted to 40° C. to which cholecalciferol (2000 IU), is added until completely dissolved. The fatty acid mixture comprising EPA:DHA triglycerides and cholecalciferol next is slowly added to the ethanol/isopropanol mixture while stirring under nitrogen. Cod fish oil is added in sufficient amounts to ensure complete dissolution of the EPA and DHA triglycerides. The fill phase formulation is sterilized by filtration using typically one or two filters of 0.2 μm porosity. The sterile filtrate is kept under N₂ overlay as it is filled into a capsule shell.

### Fill-Phase Formulation Tests

The compatibility of the soft gel capsule fill phase formulation will be determined using HPLC/GC analysis with an anastrozole tablet (Arimidex) standard reference. Samples of the soft gelatin dosage forms will be tested at time t=0, 2, and 4 weeks at 40° C. (dry). A parallel study will be performed by holding the fill phase at room temperature for seven days. The fill formulation will be visually inspected for signs of physical stability. Water and plasticizer challenge will be performed by spiking the fill phase with up to 10% water and 5% plasticizer. Dispersion properties in 0.01N HCl and pH 6.8 PBS buffer will be performed. Stability of the formulations at thermal cycling will be studied.

TABLE 5
Exemplary soft gelatin capsule composition.

| Ingredient | Quantity | Phase |
|---|---|---|
| Anastrozole | 0.5 mg, 1 mg, and | Solid phase (shell) |
| | 2 mg | |
| Fatty acid mixture EPA:DHA | 60% | Liquid phase |
| triglycerides (1.2:1) | | |
| Cholcalciferol | 2000 IU | Liquid phase |
| Ethanol:isopropanol (1:1 v/v) | 40% | Liquid phase |
| Fish oil | q.s. | Liquid phase |

### Example 4

### Gel Masses Having Various Ratios of Gelatin to Polymer and Different Ribbon Thickness

Gel masses made based on Example 3(a) where the enteric polymer and gelatin weight ratio comprise 45% by weight will be manufactured having polymer to gelatin weight ratios of 1:19 (5%), 1:9 (10%) and 1:5.7 (15%). The resulting gel masses will be cast as films and characterized for enteric properties using USP disintegration and dissolution apparatus:

### Example 5

### Dissolution (Release Rate) Tests

Release rate studies will be performed using USP dissolution paddle system at 50 rpm, 900 mL of Simulated Gastric fluid (no enzymes, “SGF”) and 900 mL of Simulated Intestinal Fluid (no enzymes “SIF”) at 37° C. A continuous flow system will be used to monitor the absorbance at 272 nm (anastrozole) at 1 min intervals over 3 hours. The capsules will be placed into cylindrical cages (1 cm diameter×3 cm long) of 20 inch stainless steel screen.

The release rate studies will be performed using a USP dissolution paddle system at 37° C. The capsules will be subjected to SGF for the first 2 hours, and then the cages will be transferred to the SIF media. Three runs for each capsule will be made and the results will be determined. The release of the drug will be determined using UPLC using a UV detector at 245 nm. No changes of in the absorbance will be indicative of preserved integrity of the coatings.

### Example 6

### Therapeutic Treatment of Hyperplasia

A patient diagnosed with hyperplasia in two breast ducts (one duct in her right breast; second duct in her left breast) will be treated with endoxifen in a transdermal composition as follows. A subject may be diagnosed with hyperplasia by any method possible, including mammography. In the following non-limiting example, the subject is diagnosed with hyperplasia using the NAF fluid. Sufficient amount of NAF will be collected from each breast of a subject. The sample from each breast will be analyzed using cytology tests and determining the expression pattern of cancer biomarkers CK5, CK14, CK7, CK18, and p53 using antibodies directed against these biomarkers. The analysis will reveal ductal hyperplastic disorder in the two suspect ducts. Each suspect duct will be fitted with a small osmostic pump which will be placed under local anesthetic into the suspect ducts through the ductal orifice and ductal lumen into the lactiferous sinus of each duct which comfortably house each osmostic pump mechanism. A small wire or thread will be left protruding through the accessed duct to identify the pump, and to provide a means to retrieve the pump at a later date. The pumps are filled with gel of Example 1. The pumps will disperse 1 mL of the gel into each duct. The pump mechanism will be tested by aspirating a small portion of the ductal fluid after pump installation to identify an expected amount of endoxifen in the ductal fluid. The ductal cells will be re-analyzed by cytology periodically (i.e., once a week, biweekly, month, etc.) by collecting NAF to determine if the endoxifen composition is effective. The NAF will also be tested for the presence of inflammation markers such as C-reactive protein (CRP), Serum Amyloid A protein (SAA), beta protein-1 (BP1) to identify if omega-3 fatty acids are effective and to decide if the dosage or the release rate needs to be increased or decreased. Serum estradiol levels and endoxifen levels will also be monitored by drawing blood and isolating plasma to determine their systemic levels.

### Example 7

### Therapeutic Treatment of a Cancerous Lesion by Infusing Breast Ducts with Fulvestrant

A post-menopausal woman who is mammographically positive and has ductal fluid in her breasts tested as described above will be treated intraductally with fulvestrant. A high-grade DCIS lesion will be identified by cytological analysis in one of her ducts. The test will be accompanied by single nucleus sequencing of a cell from NAF and will be positive for mutations in several breast cancer genes. The nipple connecting the suspect duct will be cannulated and the duct size will be estimated by using progressively bigger galactography needles to determine the lumen size of the orifice and just below the orifice. A microcatheter tube will be inserted into an identified ductal orifice under anesthesia after the orifice is dilated after the use of galactography needles. The microcatheter will be pushed into the ductal lumen until the end of the tube is flush with the nipple surface. The subject will then receive a first administration of a biodegradable fulvestrant hydroalcoholic gel (20 mg/g w/w of gel) of example 2. The amount of gel administered will be dependent on the capacity of the lactiferous sinus and the ductal lumens (typically about 1 mL), and to the best extent possible the duct will be filled with the gel. The dissipation time of the gel will be estimated based on the concentration of the active pharmaceutical ingredients and HPMC in the gel and the volume of the gel delivered to the duct. The subject will be rescheduled for a readministration of more gel at the completion of that time period. The tube will remain in place in order to facilitate the readministration (which will occur at approximately 3 to 6 weeks later).

Each subject will undergo mammography test pre- and post-treatment to measure the effect of the gel on breast density.

Blood and NAF samples will be collected pre- and during treatment at days 0, 7, 14, 21, 28, 35, 42 and 49. Plasma concentrations of estradiol, fulvestrant, for complete blood counts (CMC), bilirubin, serum glutamic-pyruvic transaminase (SGPT), serum glutamic-oxaloacetic transaminase (SGOT), alkaline phosphatase, creatinine, anastrozole, estradiol, follicle-stimulating hormone (FSH), luteinizing hormone (LH), progesterone, sex hormone-binding globulin (SHBG), cholesterol, high-density lipoprotein (HDL), low-density lipoprotein (LDL), triglycerides, fibrinogen, C-reactive protein (CRP) and anti-thrombin III will be measured for determining the safety and efficacy of the composition. NAF samples will be tested for estradiol, fulvestrant, omega-3 fatty acid, cholecalciferol, and cytokeratin expression on NAF cells.

It is expected that plasma estradiol levels will be lower than 5 ng/mL and that the composition and dose will be well tolerated by the subjects. The treatment is not expected to affect FSH, LH or estradiol or progesterone hormone levels.

### Example 8

### Therapeutic Treatment of a Cancerous Lesion by Treating with Anastrozole Oral Soft Gelatin Capsule

Thirty one female post-menopausal subjects scheduled for breast cancer surgery will be randomly assigned to 1 of 5 groups. They will receive either oral Arimidex (Astrazeneca) at 1 mg/day, or oral soft gel capsule as outlined in table 4. Treatment will be daily and will last for 4 weeks prior to surgery. The study will evaluate 3 different oral doses of anastrozole (0.5 mg, 1 mg, and 2 mg) with fixed omega-3 fatty acid and cholecalciferol doses taken orally once a day. One subject will receive placebo.

On the day of initiation of the treatment (time=day 0), each subject will undergo mammography test to set up a baseline for breast density. Blood will be drawn to establish the baseline for plasma estradiol concentration. NAF samples will be collected from each subject using a breast pump device, ForeCyte. Thereafter, blood and NAF will be drawn on days 7, 14, 21, 28 of treatment period. On the last day of the treatment, prior to surgery, each subject will undergo mammography test again to determine effect of treatment on the breast density.

During surgery, two samples (1 cm³) of breast tissue will be excised, one tumoral and the other macroscopically normal. They will be immediately frozen in liquid N₂ until assayed.

Pre- and post-treatment blood samples will be assayed for complete blood counts (CMC), bilirubin, serum glutamic-pyruvic transaminase (SGPT), serum glutamic-oxaloacetic transaminase (SGOT), alkaline phosphatase, creatinine, anastrozole, estradiol, follicle-stimulating hormone (FSH), luteinizing hormone (LH), sex hormone-binding globulin (SHBG), cholesterol, high-density lipoprotein (HDL), low-density lipoprotein (LDL), triglycerides, fibrinogen, C-reactive protein (CRP) and anti-thrombin III. NAF samples will be tested for changes in the expression of the cytokeratins and tumor markers, if any.

Pre- and post-treatment NAF samples will be used for classification of the cancer type, (for e.g., whether basal or luminal) and the pattern of expression of CK5, CK14, CK7, CK 18, and p63 on NAF cells will be determined. Concentration of anastrozole, omega-3 fatty acids, cholecalciferol and estradiol in the NAF will be measured. On days 7, 14, 21, 28 of treatment period, these tests will be repeated to monitor the effect of the drug on the subject.

Pre- and post-treatment breast density of each breast of the subjects will be determined.

Side effects such as local irritation and inflammation, dizzy spells, cystitis, vaginitis, hot flashes etc., will be monitored. It is expected that plasma estradiol levels will be lower than 5 ng/mL and that the composition and dose will be well tolerated by the subjects. The plasma anastrozole levels are expected to be detectable but its effect is expected to be reduced due to the omega-3 fatty acid and cholecalciferol. The treatment is not expected to affect FSH, LH or estradiol or progesterone hormone levels.

### Example 9

### Permeation of Endoxifen by Skin Models

This example describes various skin models for the study of endoxifen permeation.

### EpiDerm™ Skin Model

EpiDerm™ will be incubated for 1 h at 37° C. with 5% CO₂ prior to dosing. Each of the EpiDerm™ batch will be used within 3 days of delivery. An IRB approved human skin samples from operating rooms from hospitals will be obtained. The subcutaneous fat from fresh mastectomy and abdominoplasty specimens will be removed and the full-thickness skin will be immobilized to obtain split-thickness skin (STS) using a surgical blade (George Tiemann and co. Hauppauge, N.Y.) or an electric dermatome (robins instrument Inc., Chatham, N.J.). The thickness of STS samples will be measured with an electronic digital μm (Tresna Instruments co. Fuilin, China).

EpiDerm™ and STS samples will be cut into 3 mm×10 mm pieces, placed horizontally on a Cryomold, embedded in a tissue freezing medium (OCT™ compound; Tissue-Tek, Sakura Fintek USA, Torrance, Calif.), frozen in the microtome/cryostat chamber, sectioned at 7 microns and stained with Mayer's hemotoxylin and eosin. Images will be taken with a 20× objective using a Nikon Eclipse optical microscope with a 10μ bar superimposed on the skin images to measure the permeation of endoxifen into the skin.

### Porcine Skin Model

Strips of porcine sow breasts will be obtained post mortem from a local abattoir prior to cleaning procedure and transported to laboratory in iced HEPES modified Hanks buffered balanced salt solution (HBBS). The freshly excised strips of the porcine mammary papillae will be washed in tepid water and the mammary papilla surrounded by 2 cm×2 cm of abdominal skin will be excised by blunt dissection. Cutaneous fatty tissue will be removed by blunt dissection also and the pieces are maintained in HHBS until set up in diffusion cells.

The in vitro trans-mammary papilla delivery will be tested using the Franz diffusion cell system consisting of 2 separate chambers, a donor chamber and a receptor chamber. The porcine breast samples will be mounted between the cell compartments, the flanges of which will be smeared with high vacuum silicon grease with the mammary papilla located in the center and facing upwards as described by Lee et al. (International J. Pharmaceutics. 2010, 387, 161-166). The 2 chambers will be held together with a clamped to minimize leakage. The receptor chamber has a volume of about 4.3 mL and will be filled with receptor fluid via a sampling arm. Micro-stirrer bars will be added and the complete diffusion cells will be placed on a submersible magnetic stirrer base set up in a water bath at 37° C. When the breast is mounted in the horizontal position, the donor will be sealed with a greased microscope slide and the cell rotated 90° and supported s necessary. After 30 m, 500 μL of the composition will be applied to the surface of the skin by means of a pipette or a spatula in case of the hydroalcoholic gel. The donor compartment will be then occluded with laboratory film (n=4).

After 6 h, the diffusion cells will be dismantled and the breast tissue will be recovered. Excess dose and grease will be wiped away and the diffused are excised and centrifuged at 10,000×g to remove excess solution and gel, cut into approximately 1 mm×1 mm×1 mm cubes with a scalpel and placed into a 5 mL centrifuge tube. Methanol (2 mL) will be added and the tube will be vortex mixed for 30 s before being placed on a rotating blood cell mixer for 30 m. the tubes will then be centrifuged at 10,000×g and the supernatant is decanted into 10 mL glass bottles. Further aliquots of methanol will be added and the extraction process will be repeated twice more before the pooled supernatants will be reduced in a vacuum oven set at 50° C. The residues will be then reconstituted with a 1 mL of HPLC mobile phase.

Samples will be analyzed by reverse-phase liquid chromatography. Analytes will be separated and statistical analyses will be performed on the results. Drug delivery into the papilla oriented vertically will be compared with that delivered into the papilla oriented laterally by means of Wilcoxon match-pairs signed-ranks test, and the combined formulation of the samples will be compared via a Kruskal-Wallis non-parametric ANAOVA test (Instat 3, GraphPad software, CA, USA). Confidence intervals will be set at 95% and p<0.05 is deemed as statistically significant.

### Example 10

### In Vitro Skin Permeation Test Method

This example describes a method for obtaining skin permeation data according to the following test method.

When a transdermal delivery device is evaluated, the release liner will be typically removed from a 2.0 cm² patch and the patch will be applied to a human cadaver skin and pressed to cause uniform contract with the skin. The resulting patch/skin laminate will be placed patch side up across the orifice of the lower portion of a vertical diffusion cell. The diffusion cell will be assembled and the lower portion will be filled with 10 mL of warm (32° C.) receptor fluid (0.1 M PBS, pH 6.8) so that the receptor fluid contacts the skin. The sampling port will be covered when in use.

The cells will be maintained at 32±2° C. throughout the course of the experiment. The receptor fluid will be stirred by means of a magnetic stirrer throughout the experiment to assure uniform sample, and a reduced diffusion barrier on the dermal side of the skin. The entire volume of the receptor fluid will be withdrawn at specified time intervals and immediately replaced with fresh fluid. The withdrawn fluid will be filtered through a 0.45 μfilter. The last 1-2 mL will be then analyzed for the active ingredients, 4-OHT, desmethyltamoxifen, endoxifen, fulvestrant, and anastrozole. Using HPLC methods (Column: Phenomenex Spherex, 75×4.6 mm, 3 μm particle size; mobile phase: 400:200:400 Methanol:Acetonitrile:Buffer. Buffer will be ammonium acetate solution adjusted to pH 6.6 with acetic acid; Flow rate: 2 mL/min; Detector: UV at 230 nm; Injection volume: 10 μL; run time: 1.9 m). The cumulative amount of the active ingredients penetrating through the skin will be calculated and reported as μg/cm².

### Example 11

### Preparation of Endoxifen Patch

An endoxifen patch may be prepared as follows: A cation salt of endoxifen will be added to an organic or lipophilic solvent such as ethanol, isopropanol, ethyl acetate, methanol, acetone, 2-butanone, toluene, alkanes, and mixtures thereof while stirring until all of endoxifen is dissolved. The dissolved therapeutic agent is added to a fatty acid mixture comprising EPA triglyceride and mixed. To this mixture 6000 IU of cholecalciferol is added while stirring under N₂ overlay. To this solution, a copolymer such as SIS block, a permeation enhancer such as ISM provide a coating composition. The mixture is shaken or stirred until a homogenous coating composition is obtained. The resulting composition is then applied to a release liner using conventional coating methods (e.g., knife coating or extrusion die coating) to provide a predetermined uniform thickness of coating composition and dried to remove all traces of solvent. The release liner is then laminated onto a polyurethane backing layer.

### Example 12

### Patch Stability Methods

Endoxifen patches (20 cm2) will be sealed in pouches (BAREX™/aluminium/polyester or BAREX™/aluminum/paper laminates and stored under conditions of 25° C./60% relative humidity and 40° C./75% relative humidity. The patches will be tested for their drug content and/or their probe tack before storage and after preset storage times of 1 m, 2 m, 3 m, 6 m, 1 yr, and 2 yrs using test methods described below.

### Drug Content Test Method

The drug content test data will be obtained using the following test method. The liner will be removed from the patches and the patches will be placed in a 120 mL jar. The backing and coating will be extracted using 75 mL of a solution consisting of 75:25 by volume tetrahydrofuran (THF): methanol (MeOH). The samples will be shaken overnight. Dilutions of the samples will be prepared by placing 10 mL of the resulting solutions into 44 mL vials and adding 30 mL of the additional THF:MeOH to each vial. Aliquots of these final dilutions will then be placed in autsampler vials for analysis by Gas chromatography with flame ionization detection (GC-FID) using a J&W DB-5 fused silica capillary column (15 m×0.53 mm i.d., 1.5 μm film of (5%-Phenyl)-methylpolysiloxane) with helium carrier agas.

### Probe Tack Test Method

The tack data will be obtained using a Digital Polyken Probe Tack Test, Model 80-02-01 (Testing Machines Inc., Amityville, N.Y.). The machine settings will be as follows: speed: 0.5 cm/second, dwell: 2 seconds; mode: peak. A stainless steel prove will be used. The result of the test will the force required to break the bond between the probe and the surface of the test sample. The force will be measured in “grams of tack”.

### Peel Adhesion to Vitro-Skin Method

The peel adhesion data will be obtained using the following test method. The peel adhesion testing will be based on ASTM D3330-90. This will involve peel from a substrate at a 180° peel angle done with a constant rate of extension tensile tester. The substrate used will be Vitro-Skin™ N-19 (VS), an artificial skin substitute available from Innovative Measurement Solutions, Inc. that is designed to mimic human back skin. The VS may be conditioned prior to sue at 23° C. for about 16 hours in a chamber containing a solution consisting 70:30 by volume water:glycerol to maintain constant humidity. All testing will be done on the textured side of the VS. Immediately upon removal of the VS from the conditioning chamber, the VS will be attached using a double sided adhesive tape to the backing of a foam tape (3M 1777, 40 mil (1016 μm) thick) which will be attached to a steel plate to provide a stable testing surface. Testing will be done in a controlled environment at 23° C.±2° C. and 50%±3% relative humidity. A 1.0 cm width strip of the coated sheet will be allowed to dwell for 2 m prior to peel testing.

The free end of the coated strip will be doubled back so the angle of removal will be 180°. The removal peel rate will be determined and the force of adhesion will be presorted as grams per centimeter (g/cm).

### Example 13

### Therapeutic Treatment of a Cancerous Lesion by Treating with Endoxifen Transdermal Patch

A reservoir patch containing 0.86 gram of a formulation including a cationic salt of endoxifene as described in example 11 will be tested on 12 human subjects. The reservoir patch will have a skin-formulation contact area of 6 cm². The patch will be applied to the breast nipple and the areola. The patch will be applied to a subject's skin for 72 h before removal. In some subjects, a second patch may be placed after 72 h on a different location on the breast for additional 72 h before removal of the second patch.

The mean plasma concentration of endoxifen following the patch application will be determined. The mean Area Under the Plasma Drug Concentration Curve (AUC) values or 0-72 h and 0-144 h will be determined and expressed as ng-h/mL.

The foregoing description is given for clearness of understanding only, and no unnecessary limitations should be understood therefrom, as modifications within the scope of the invention will be readily apparent to one of skill in the art.

Throughout the specification and the claims which follow, where the compositions are described as including components or materials, it is contemplated that the composition can also consist essentially of or consist of any combination of the recited components or materials unless expressly limited otherwise. Likewise, where methods are described as including particular steps, it is contemplated that the methods can also consist essentially of, or consist of any combination of the recited steps and in any order of the recited steps that is will result in same or similar compositions, unless expressly limited otherwise. The invention illustratively disclosed herein suitably may be practiced in the absence of any element or step which is not specifically disclosed herein.

References are made in detail to certain embodiments of the invention, examples of which are illustrated herein. It is intended that any and all parts of the disclosure may be read in combination with any other part of the disclosure, unless otherwise apparent from the text. While the invention is described in conjunction with the enumerated embodiments, it will be understood that they are not intended to limit the invention to those embodiments. It is specifically intended to cover all alternatives, modifications, and equivalents which maybe included within the scope of the present invention as defined by the claims. At various places in the present specification, substituents of compounds of the invention may be disclosed in groups. It is specifically intended that the invention include each and every individual sub-combination of the members of such groups.

It is further appreciated that certain features of the invention, which are, for clarity, described in the context of separate embodiments, can also be provided in combination in a single embodiment. Conversely, various features of the invention which are, for brevity, described in the context of a single embodiment can also be provided separately or in any suitable sub-combination.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compounds and compositions disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A pharmaceutical composition for treatment of a subject in risk for or having a breast disorder or an estrogen-related disorder comprising:
i. at least one therapeutic agent;
ii. a fatty acid mixture comprising at least one omega-3 fatty acid; and
iii. at least one vitamin D compound;
wherein the composition is capable of being delivered locally to a tissue.

2. The composition according to claim 1, wherein the at least one therapeutic agent is a SERM, a SERD, an AI, or a combination thereof, and pharmaceutically acceptable salts thereof.

3. The composition according to claim 2, wherein the SERM is selected from the group consisting of tamoxifen, cis-tamoxifen, 4-OHT, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923.

4. The composition according to claim 2, wherein the SERM is 4-OHT, desmethyltamoxifen, or endoxifen.

5. The composition according to claim 2, wherein the SERD is a fulvestrant, ARN-810, or CH4986399.

6. The composition according to claim 2, wherein the SERD is fulvestrant.

7. The composition according to claim 2, wherein the AI is selected from the group consisting of anastrozole, exemestane and letrozole.

8. The composition according to claim 2, wherein the AI is anastrozole.

9. The composition according to claim 1, wherein the at least one therapeutic agent is between 0.01% to 15% by weight of the composition.

10. The composition according to claim 1, wherein the at least one omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

11. The composition according to claim 1, wherein the omega-3 fatty acid is a triglyceride or a phospholipid.

12. The composition according to claim 1, wherein the fatty acid mixture comprising at least one omega-3 fatty acid is between 10% to 90% by weight of the composition.

13. The composition according to claim 1, wherein the fatty acid mixture comprises from 400 mg/g to 600 mg/g of the at least one omega-3 fatty acid.

14. The composition according to claim 1, wherein the fatty acid mixture comprises a plurality of omega-3 fatty acids.

15. The composition according to claim 14, wherein the fatty mixture comprises a mixture of EPA and DHA.

16. The composition according to claim 1, wherein the fatty acid mixture is a fatty acid oil mixture.

17. The composition according to claim 16, wherein the fatty acid oil mixture is derived from at least one oil selected from a group consisting of a marine oil, a plant-based oil, an algae oil, a microbial oil, and a combination thereof.

18. The composition according to claim 17, wherein the marine oil is a fish oil.

19. The composition according to claim 1, the fatty acid mixture is an emulsion.

20. The composition according to claim 19, the emulsion is an alcohol-in oil emulsion, an oil-in-alcohol emulsion, oil-in-water emulsion, water-in-oil emulsion, water-in-oil-in-water emulsion, or an oil/alcohol/water emulsion.

21. The composition according to claim 1, wherein the at least one vitamin D compound is selected from the group consisting of calciferol, cholecalciferol, ergocalciferol, vitamin D metabolites, 25-hydroxyvitamin D3, 25-hydroxyvitamin D2, 25(OH)D, 1,25(OH)(2)D, 25-hydroxyvitamin D4, 25-hydroxyvitamin D5, 25-hydroxyvitamin D7, 1-alpha-25-hydroxyvitamin D3, 1-alpha-25-hydroxyvitamin D2, 1-alpha-25-hydroxyvitamin D4,1,25-dihydroxy-19-nor-vitamin D2, 1-alphahydroxyvitamin D3, vitamin D analogs, and a combination thereof.

22. The composition according to claim 1, wherein the at least one vitamin D compound is cholecalciferol.

23. The composition according to claim 1, wherein the at least one vitamin D compound is partly or wholly dissolved, dispersed, or suspended in the fatty acid mixture comprising the at least one omega-3 fatty acid.

24. The composition according to claim 23, wherein the at least one therapeutic agent and the at least one vitamin D compound are partly or wholly dissolved, dispersed or suspended in the fatty acid mixture comprising the at least one omega-3 fatty acid.

25. The composition according to claim 1, wherein the at least one vitamin D compound has an activity ranging between 10 IU-6000 IU.

26. The composition according to claim 1, wherein the composition further comprises an excipient.

27. The composition according to claim 26, wherein the composition is formulated in a gel, a solution, a lotion, an ointment, a cream, or an emulsion.

28. The composition according to claim 27, wherein the gel comprises a vehicle, co-solvent, a stabilizing agent, a neutralization agent, a permeation enhancer, an absorption enhancer, a surfactant, a gelling agent, a polymer, a co-polymer, a cross-linking agent, an antioxidant, a moisturizer, an antimicrobial, a preservative, or a combination thereof.

29. The composition according to claim 28, wherein the vehicle is an oily vehicle.

30. The composition according to claim 29, wherein the oily vehicle is fish oil.

31. The composition according to claim 28, wherein the gelling agent is HPMC, CMC, Carbopol or polyacrylic acid.

32. The composition according to claim 28, wherein the permeation enhancer is an ether, a sulfoxide, a poloxomer, a pyrrolidone, an azone, or a fatty alcohol.

33. The composition according to claim 28, wherein the surfactant is SDS, cetrimide, Capmul, Cremaphor, or Tween 85.

34. The composition according to claim 28, wherein the antioxidant is alpha-tocopherol, BHA, BHT, ascorbic acid and pharmaceutically acceptable salts and esters thereof, propyl gallate, citric acid and pharmaceutically acceptable salts thereof, malic acid and pharmaceutically acceptable slats thereof, and sulfite salts and mixtures thereof.

35. The composition according to claim 1, further comprising at least one additional medication.

36. The composition according to claim 35, wherein the at least one additional medication is selected from the group consisting of alkylating agents, anti-neoplastics, anti-mimetics, anti-metabolites, anti-tumor antibiotics, topoisomerase inhibitors, mitotic inhibitors, cortico steroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof.

37. The composition according to claim 35, wherein the at least one additional medication is trastuzumab.

38. A pharmaceutical composition for treatment of a subject in risk for or having a breast disorder or an estrogen-related disorder comprising:
i. 0.01 g to 15 g of at least one therapeutic agent;
ii. 1 g to 10 g of the fatty acid mixture comprising at least one omega-3 fatty acid;
iii. 10 IU to 6000 IU of the at least one vitamin D compound; and
iv. a fish oil (qs) to 100 g;
wherein the composition is capable of being delivered locally to a tissue.

39. A pharmaceutical composition for treatment of a subject in risk for or having a breast disorder or an estrogen-related disorder comprising:
i. 0.01% to 15% of a SERM, a SERD, an AI or a combination thereof, or pharmaceutically acceptable salts thereof;
ii. 10% to 90% of a fatty acid mixture comprising at least one omega-3 fatty acid;
iii. 10 IU to 6000 IU of at least one vitamin D compound or pharmaceutically acceptable salts thereof; and
iv. 10% to 90% vehicle.
wherein the composition is capable of being delivered locally to a tissue.

40. The composition according to claim 39, further comprising at least one gelling agent.

41. The composition according to claim 40 wherein the at least one gelling agent is 0.1% to 80% w/w of the composition.

42. The composition according to claim 38 or 39, wherein the SERM is selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652, ERA-923, and pharmaceutically acceptable salts thereof.

43. The composition according to claim 38 or 39, wherein the SERD is selected from a group consisting of fulvestrant, ARN-810, CH4986399, and pharmaceutically acceptable salts thereof.

44. The composition according to claim 38 or 39, wherein the AI selected from a group consisting of anastrozole, exemestane, letrozole, and pharmaceutically acceptable salts thereof.

45. The composition according to claim 1, wherein the composition is delivered using a transdermal, a transpapillary, or an intraductal device.

46. A device according to claim 45, wherein a transdermal device is selected from the group consisting of an applicator, a patch, a tape, sheet, a dressing, a spray device and an aerosolizer.

47. A device according to claim 46, wherein the patch for transdermal delivery comprises:
i. a backing layer; and
ii. an adhesive layer having a skin-contacting adhesive surface;
wherein the adhesive layer comprises a drug reservoir comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

48. A device according to claim 46, wherein the patch for transdermal delivery comprises:
i. a backing layer;
ii. a drug reservoir disposed on a first layer; and
iii. a skin-contacting second layer comprising a pressure sensitive adhesive layer;
wherein the second layer is attached to a surface of the first layer opposed to a surface in contact with the backing layer, wherein the second layer is a rate-controlling layer and wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

49. A device according to claim 46, wherein the patch for transdermal delivery comprises:
i. a backing layer;
ii. a drug reservoir disposed on a first layer;
iii. a second layer comprising a rate-controlling membrane, the membrane being attached to a surface of the first layer opposed to a surface in contact with the backing layer; and
iv. a skin-contacting third layer comprising a pressure sensitive adhesive attached to a surface of the membrane that is opposed to the surface of the rate-controlling membrane in contact with the first layer;
wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

50. An oral pharmaceutical composition for treatment of a subject in risk for or having a breast disorder or an estrogen-related disorder comprising:
i. at least one therapeutic agent;
ii. a fatty acid mixture comprising at least one omega-3 fatty acid; and
iii. at least one vitamin D compound.

51. The composition according to claim 50, wherein the composition is a capsule, a caplet, and a tablet.

52. The composition according to claim 51, wherein the capsule is a gelatin capsule, gelatin-free capsule, a cap-in-cap capsule, alginate capsule, an HPMC capsule, a PVA capsule, and a seamless capsule.

53. The composition according to claim 51, wherein the capsule is a hard capsule or a soft capsule.

54. A capsule for oral delivery, the capsule comprising:
i. a shell comprising 0.1 mg to 500 mg of a SERM, a SERD, an AI, or a combination thereof; and
ii. a fill phase comprising (a) 20% to 60% of a fatty acid mixture comprising at least one omega-3 fatty acid triglyceride or phospholipid; and (b) 10 IU to 6000 IU of at least one vitamin D compound.

55. A soft gelatin capsule comprising:
i. a shell comprising 0.5 mg or 1 mg of anastrozole;
ii. a fill phase comprising (a) 60% of a fatty acid mixture comprising at least 99% EPA triglyceride or phospholipid; and (b) 400 IU of cholecalciferol; and
iii. a sufficient amount of fish oil.

56. The composition according to claim 1 or claim 50, wherein the composition comprises a first composition comprising the fatty acid mixture comprising the at least one omega-3 fatty acid, a second composition comprising the at least one vitamin D compound, and a third composition comprising the at least one therapeutic agent.

57. The composition according to claim 1 or claim 50, wherein the composition comprises a first composition comprising the fatty acid mixture comprising the at least one omega-3 fatty acid and the at least one vitamin D compound and a second composition comprising the at least one therapeutic agent.

58. The composition according to claim 1 or claim 50, wherein the composition comprises a single composition comprising the fatty acid mixture comprising the at least one omega-3 fatty acid, the at least one vitamin D compound and the at least one therapeutic agent.

59. A method of preparing a pharmaceutical composition, comprising mixing:
i. at least one therapeutic agent;
ii. a fatty acid mixture comprising at least one omega-3 fatty acid;
iii. at least one vitamin D compound;
iv. optionally, an excipient; and
v. optionally, at least one additional medication.

60. A method according to claim 59, wherein the composition is formulated for local delivery.

61. A method preparing a pharmaceutical composition, comprising the steps of:
i. providing an amount of at least one therapeutic agent;
ii. providing an amount of a fatty acid mixture comprising at least one omega-3 fatty acid;
iii. providing an amount of at least one vitamin D compound;
iv. providing at least one excipient;
v. combining the fatty acid mixture comprising at least one omega-3 fatty acid, the at least one vitamin D compound, and the at least one excipient, thereby forming a fill phase; and
vi. encapsulating the fill phase in a shell,
wherein the at least one therapeutic agent is comprised in the fill phase or the shell or both.

62. A method preparing a pharmaceutical composition, comprising the steps of:
i. providing an amount to a fatty acid oil mixture comprising EPA and DHA at a weight ratio ranging from 1:10 to 10:1;
ii. providing at least one vitamin D compound in the fatty acid oil mixture comprising the EPA and DHA;
iii. encapsulating the fatty acid oil mixture in a shell;
iv. providing the shell with a coating; and
v. providing an amount of anastrozole in the coating of the shell.

63. A method according to claim 61 or claim 62, wherein the capsule is a hard capsule or a soft capsule.

64. A method according to claim 61 or claim 62, wherein the capsule shell is prepared using a plate process, a rotary die process, or a reciprocating die process.

65. A method according to claim 61 or 62, wherein the method further comprises providing at least one additional medication in the shell or the fill phase or both.

66. A method for treatment of a subject at risk for or having a breast disorder or an estrogen-related disorder, the method comprising administering to the subject a pharmaceutical composition comprising:
i. at least one therapeutic agent;
ii. a fatty acid mixture comprising at least one omega-3 fatty acid; and
iii. at least one vitamin D compound.

67. The method according to claim 66, wherein the at least one therapeutic agent is a SERM, a SERD, an AI, or a combination thereof, and pharmaceutically acceptable salts thereof.

68. The method according to claim 67, wherein the SERM is selected from the group consisting of tamoxifen, cis-tamoxifen, 4-OHT, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923.

69. The method according to claim 67, wherein the SERM is 4-OHT, desmethyltamoxifen, or endoxifen.

70. The method according to claim 67, wherein the SERD is a fulvestrant, ARN-810, or CH4986399.

71. The method according to claim 67, wherein the SERD is fulvestrant.

72. The method according to claim 67, wherein the AI is selected from the group consisting of anastrozole, exemestane and letrozole.

73. The method according to claim 67, wherein the AI is anastrozole.

74. The method according to claim 66, wherein the at least one therapeutic agent is between 0.01% to 15% by weight of the composition.

75. The method according to claim 66, wherein the at least one omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

76. The method according to claim 66, wherein the omega-3 fatty acid is a triglyceride or a phospholipid.

77. The method according to claim 66, wherein the fatty acid mixture comprising at least one omega-3 fatty acid is between 10% to 90% by weight of the composition.

78. The method according to claim 66, wherein the fatty acid mixture comprises from 400 mg/g to 600 mg/g of the at least one omega-3 fatty acid.

79. The method according to claim 66, wherein the fatty acid mixture comprises a plurality of omega-3 fatty acids.

80. The method according to claim 79, wherein the fatty mixture comprises a mixture of EPA and DHA.

81. The method according to claim 66, wherein the fatty acid mixture is a fatty acid oil mixture.

82. The method according to claim 81, wherein the fatty acid oil mixture is derived from at least one oil selected from a group consisting of a marine oil, a plant-based oil, an algae oil, and a microbial oil.

83. The method according to claim 82, wherein the marine oil is a fish oil.

84. The method according to claim 66, the fatty acid mixture is an emulsion.

85. The method according to claim 84, the emulsion is an alcohol-in oil emulsion, an oil-in-alcohol emulsion, oil-in-water emulsion, water-in-oil emulsion, water-in-oil-in-water emulsion, or an oil/alcohol/water emulsion.

86. The method according to claim 66, wherein the at least one vitamin D compound is selected from the group consisting of calciferol, cholecalciferol, ergocalciferol, vitamin D metabolites, 25-hydroxyvitamin D3, 25-hydroxyvitamin D2, 25(OH)D, 1,25(OH)(2)D, 25-hydroxyvitamin D4, 25-hydroxyvitamin D5, 25-hydroxyvitamin D7, 1-alpha-25-hydroxyvitamin D3, 1-alpha-25-hydroxyvitamin D2, 1-alpha-25-hydroxyvitamin D4,1,25-dihydroxy-19-nor-vitamin D2, 1-alphahydroxyvitamin D3, vitamin D analogs, and a combination thereof.

87. The method according to claim 66, wherein the at least one vitamin D compound is cholecalciferol.

88. The method according to claim 66, wherein the at least one vitamin D compound is partly or wholly dissolved, dispersed, or suspended in the fatty acid mixture comprising the at least one omega-3 fatty acid.

89. The method according to claim 88, wherein the at least one therapeutic agent and the at least one vitamin D compound are partly or wholly dissolved, dispersed or suspended in the fatty acid mixture comprising the at least one omega-3 fatty acid.

90. The method according to claim 66, wherein the at least one vitamin D compound has an activity ranging between 10 IU-6000 IU.

91. The method according to claim 66, wherein the composition further comprises an excipient.

92. The method according to claim 91, wherein the composition is formulated in a gel, a solution, a lotion, an ointment, a cream, or an emulsion.

93. The method according to claim 92, wherein the gel comprises a vehicle, co-solvent, a stabilizing agent, a neutralization agent, a permeation enhancer, an absorption enhancer, a surfactant, a gelling agent, a polymer, a co-polymer, a cross-linking agent, an antioxidant, a moisturizer, an antimicrobial, a preservative, or a combination thereof.

94. The method according to claim 93, wherein the vehicle is an oily vehicle.

95. The method according to claim 94, wherein the oily vehicle is fish oil.

96. The method according to claim 93, wherein the gelling agent is HPMC, CMC, Carbopol or polyacrylic acid.

97. The method according to claim 93, wherein the permeation enhancer is an ether, a sulfoxide, a poloxomer, a pyrrolidone, an azone, or a fatty alcohol.

98. The method according to claim 93, wherein the surfactant is SDS, cetrimide, Capmul, Cremaphor, or Tween 85.

99. The method according to claim 93, wherein the antioxidant is alpha-tocopherol, BHA, BHT, ascorbic acid and pharmaceutically acceptable salts and esters thereof, propyl gallate, citric acid and pharmaceutically acceptable salts thereof, malic acid and pharmaceutically acceptable slats thereof, and sulfite salts and mixtures thereof.

100. The method according to claim 66, further comprising at least one additional medication.

101. The method according to claim 100, wherein the at least one additional medication is selected from the group consisting of alkylating agents, anti-neoplastics, anti-mimetics, anti-metabolites, anti-tumor antibiotics, topoisomerase inhibitors, mitotic inhibitors, cortico steroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof.

102. The method according to claim 100, wherein the at least one additional medication is trastuzumab.

103. A method for treatment of a subject at risk for or having a breast disorder or an estrogen-related disorder, the method comprising administering to the subject a pharmaceutical composition comprising:
i. 0.01 g to 15 g of at least one therapeutic agent;
ii. 1 g to 10 g of the fatty acid mixture comprising at least one omega-3 fatty acid;
iii. 10 IU to 6000 IU of the at least one vitamin D compound; and
iv. a fish oil (qs) to 100 g;
wherein the composition is capable of being delivered locally to a tissue.

104. A method for treatment of a subject at risk for or having a breast disorder or an estrogen-related disorder, the method comprising administering to the subject a pharmaceutical composition comprising:
i. 0.01% to 15% of a SERM, a SERD, an AI or a combination thereof, or pharmaceutically acceptable salts thereof;
ii. 10% to 90% of a fatty acid mixture comprising at least one omega-3 fatty acid;
iii. 10 IU to 6000 IU of at least one vitamin D compound or pharmaceutically acceptable salts thereof; and
iv. 10% to 90% vehicle.
wherein the composition is capable of being delivered locally to a tissue.

105. The method according to claim 104, further comprising at least one gelling agent.

106. The method according to claim 105, wherein the at least one gelling agent is 0.1% to 80% w/w of the composition.

107. The method according to claim 104, wherein the SERM is selected from a group consisting of tamoxifen, cis-tamoxifen, endoxifen, 4-hydroxytamoxifen, desmethyltamoxifen, lasofoxifene, raloxifene, benzothiphene, bazedofoxifene, arzoxifene, miproxifene, levormeloxifene, droloxifene, clomifene, idoxifene, toremifene, EM652 and ERA-923 and pharmaceutically acceptable salts thereof.

108. The method according to claim 104, wherein the SERD is selected from a group consisting of fulvestrant, ARN-810, and CH4986399 and pharmaceutically acceptable salts thereof.

109. The method according to claim 104, wherein the AI selected from a group consisting of anastrozole, exemestane, and letrozole, and pharmaceutically acceptable salts thereof.

110. The method according to claim 66, wherein the composition is delivered using a transdermal, a transpapillary, or an intraductal device.

111. A method according to claim 110, wherein the transdermal device is selected from the group consisting of an applicator, a patch, a tape, sheet, a dressing, a spray device and an aerosolizer.

112. A method according to claim 111, wherein the patch for transdermal delivery comprises:
i. a backing layer; and
ii. an adhesive layer having a skin-contacting adhesive surface;
wherein the adhesive layer comprises a drug reservoir comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

113. A method according to claim 111, wherein the patch for transdermal delivery comprises:
i. a backing layer;
ii. a drug reservoir disposed on a first layer; and
iii. a skin-contacting second layer comprising a pressure sensitive adhesive layer;
wherein the second layer is attached to a surface of the first layer opposed to a surface in contact with the backing layer, wherein the second layer is a rate-controlling layer and wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

114. A method according to claim 111, wherein the patch for transdermal delivery comprises:
i. a backing layer;
ii. a drug reservoir disposed on a first layer;
iii. a second layer comprising a rate-controlling membrane, the membrane being attached to a surface of the first layer opposed to a surface in contact with the backing layer; and
iv. a skin-contacting third layer comprising a pressure sensitive adhesive attached to a surface of the membrane that is opposed to the surface of the rate-controlling membrane in contact with the first layer;
wherein the drug reservoir comprises a composition comprising (a) at least one therapeutic agent, (b) a fatty acid mixture comprising at least one omega-3 fatty acid, and (c) at least one vitamin D compound sufficient to treat a breast disorder or estrogen-related disorder for at least three days.

115. A method according to claim 110, wherein the method comprises:
i. delivering the composition to a breast duct of the subject, comprising contacting the composition contained within a treatment chamber of a device with a nipple of a breast; and
ii. applying positive pressure on the composition.

116. A method according to claim 110, wherein the method comprises delivering the composition to a breast duct of a subject using a device selected from the group consisting of syringe and needle, microneedles, catheters, microcatheters, beads, and microbeads.

117. A method according to claim 110, wherein the method comprises delivering the composition to a breast duct of a subject, comprising placing in the breast duct a breast duct device comprising an indwelling reservoir capable of dwelling in the breast duct, and optionally, a line or tube connected to the reservoir to reload the indwelling reservoir when empty, or to provide retrieval of the indwelling reservoir from the breast duct, wherein the composition released to the breast duct.

118. A method for treatment of a subject at risk for or having a breast disorder or an estrogen-related disorder, the method comprising for administering to the subject an oral pharmaceutical composition comprising:
i. at least one therapeutic agent;
ii. a fatty acid mixture comprising at least one omega-3 fatty acid; and
iii. at least one vitamin D compound.

119. The method according to claim 118, wherein the at least one therapeutic agent is tamoxifen, cis-tamoxifen, endoxifen, 4-OHT, desmethyltamoxifen, 4-hydroxy-N-desmethyl tamoxifen, lasofoxifene, raloxifene, benzothiphene, bazedofoxifene, arzoxifene, miproxifene, levormeloxifene, droloxifene, clomifene, idoxifene, toremifene, EM652, ERA-923, fulvestrant, ARN-810, CH4986399, anastrozole, exemestrane, and letrozole.

120. The method according to claim 118, wherein the composition is formulated in a capsule, a caplet, and a tablet.

121. The method according to claim 118, wherein the capsule is a gelatin capsule, gelatin-free capsule, a cap-in-cap capsule, alginate capsule, an HPMC capsule, a PVA capsule, and a seamless capsule.

122. The method according to claim 118, wherein the capsule is a hard capsule or a soft capsule.

123. The method according to claim 118, wherein the capsule comprises:
i. a shell comprising 0.1 mg to 500 mg of a SERM, a SERD, an AI, or a combination thereof; and
ii. a fill phase comprising (a) 20% to 60% of a fatty acid mixture comprising at least one omega-3 fatty acid triglyceride or phospholipid; and (b) 10 IU to 6000 IU of at least one vitamin D compound.

124. The method according to claim 121, wherein the soft capsule comprises:
i. a shell comprising 0.5 mg or 1 mg of anastrozole;
ii. a fill phase comprising (a) 60% of a fatty acid mixture comprising at least 99% EPA triglyceride or phospholipid; and (b) 400 IU of cholecalciferol; and
iii. a sufficient amount of fish oil.

125. The method according to claim 118, wherein the composition further comprises at least one additional medication.

126. The method according to claim 125, wherein the at least one additional medication is selected from the group consisting of alkylating agents, anti-neoplastics, anti-mimetics, anti-metabolites, anti-tumor antibiotics, topoisomerase inhibitors, mitotic inhibitors, cortico steroids, differentiating agent, anti-cancer antibodies, immunotherapy agents, anthracyclins, platinums, vinca alkoids, camptothecins, hormones, 1-alpha-hydroxylase inhibitors, 24-hydroxylase inhibitors, or a combination thereof.

127. The method according to claim 126, wherein the at least one additional medication is trastuzumab.

128. The method according to claim 66, wherein the breast disorder is a proliferative breast disease, a breast cancer, a breast scarring, or an increase in breast density.

129. The method according to claim 128, wherein the breast cancer is ductal carcinoma in situ (DCIS), microinvasive breast carcinoma (MIC), lobular carcinoma in situ (LCIS), invasive (or infiltrating) lobular carcinoma (ILC), invasive ductal carcinoma (DC), or inflammatory breast cancer, ER-positive breast cancer, ER-negative breast cancer, triple negative breast cancer (TNBC), adenoid cystic (adenocystic) carcinoma, law-grade adenosquamatous carcinoma, medullary carcinoma, mucinous (or colloid) carcinoma, papillary carcinoma, tubular carcinoma, metaplastic carcinoma, and micropapillary carcinoma.

130. The method according to claim 128, wherein the proliferative beast disease is a mild hyperplasia, hyperplasia of the usual type, atypical ductal hyperplasia, and atypical lobular hyperplasia.

131. The method according to claim 128, wherein breast cancer is ER+ metastatic breast cancer, ER+ refractory breast cancer, AR+/ER+ breast cancer, AR+/ER+ refractory breast cancer, AR+/ER+ metastatic breast cancer, and triple positive breast cancer.

132. The method according to claim 66, wherein composition administered to a subject delivers a daily dose of the at least one therapeutic agent ranging from 0.1 mg/breast to 250 mg/breast, from 0.1 mg/breast to 200 mg/breast, from 0.1 mg/breast to 150 mg/breast, from 0.1 mg/breast to 100 mg/breast, 0.1 mg/breast to 50 mg/breast, from 0.5 mg/breast to 50 mg/breast, from 5 mg/breast to 45 mg/breast, from 5 mg/breast to 40 mg/breast, from 5 mg/breast to 35 mg/breast, from 5 mg/breast to 30 mg/breast, from 5 mg/breast to 25 mg/breast, from 5 mg/breast to 20 mg/breast, from 5 mg/breast to 15 mg/breast, from 5 mg/breast to 10 mg/breast, from 1 mg/breast to 25 mg/breast, from 2 mg/breast to 20 mg/breast, from 3 mg to 30 mg/breast, and from 4 mg/breast to 40 mg/breast.

133. The method according to claim 66, wherein the subject is administered a daily dose of the endoxifen of 0.25 mg/breast, 0.75 mg/breast, 1 mg/breast, or 2 mg/breast.

134. The method according to claim 66, wherein the subject is administered a daily dose of fulvestrant administered of 0.25 mg/breast, 1 mg/breast, 5 mg/breast, 10 mg/breast, 20 mg/breast, 25 mg/breast, 30 mg/breast, 35 mg/breast or 40 mg/breast.

135. The method according to claim 66, wherein the dose of the at least one therapeutic agent administered to the subject per breast duct is 0.25 mg/g, 0.5 mg/g, 1 mg/g, 2 mg/g, 5 mg/g, 10 mg/g and 20 mg/g by weight of gel.

136. The method according to claim 66, wherein an intraductal administration of the composition to a subject delivers the at least one therapeutic agent at a dose of 20 mg/mL per breast duct.

137. The method according to claim 66, where the composition is administered intraductally in a volume ranging from 0.1 mL to 2 mL, 0.1 mL to 1.5 mL, and 0.5 mL to 1 mL.

138. The method according to claim 66, wherein a blood or plasma concentration of the at least one therapeutic agent is less than 50 ng/ml for at least 3 days.

139. A method for treatment of a subject at risk for or having a breast disorder or an estrogen-related disorder, the method comprising:
i. collecting a NAF sample from the subject:
ii. testing the NAF sample using a testing method;
iii. determining subject's breast condition;
iv. based on subject's breast condition, administering to the subject an amount of a pharmaceutical composition comprising (a) at least one therapeutic agent; (b) a fatty acid mixture comprising at least one omega-3 fatty acid; and (c) at least one vitamin D compound.

140. A method according to claim 139, wherein the testing is selected from a group consisting of determining presence or absence of gene mutations, single nucleotide polymorphisms variations, gene copy number variations, DNA copy number variations, alterations in DNA methylation patterns, changes in histone methylation patterns, changes in micro-RNA patterns, altered micro-biome, altered cytology, and altered expression of cancer biomarkers.

141. The method according to claim 139, wherein testing comprises conducting:
(a) cytology tests; (b) a single nucleus sequencing; (c) single cell sequencing; (d) microarray analysis; (e) PCR analysis; (t) immunohistochemistry; (g) immunofluorescence; (h) and 16S RNA sequencing; and (i) RFLP.

142. A method according to claim 141, wherein the sequencing comprises dideoxy sequencing, Sanger sequencing, next generation sequencing, single molecule real time sequencing, whole genome sequencing, exome sequencing, and RNA sequencing.

143. The method according to claim 139, wherein the testing comprises:
i. collecting a NAF sample from the subject;
ii. contacting a cell of the NAF sample adsorbed to an adsorbent paper comprising antibodies that bind to CK5, CK14, CK7, CK18, and p63;
iii. detecting binding of one or more of the antibodies to said cell; and
iv. classifying the breast condition based upon the binding pattern of the antibodies.

144. A method for treatment of a subject at risk for or having a breast disorder or an estrogen-related disorder, the method comprising:
i. collecting a NAF sample from the subject;
ii. providing at least one cell from the NAF sample;
iii. conducting a whole-genome sequencing of the cell;
iv. determining the subject's risk for, presence or reoccurrence of breast disorder or estrogen-related disorder; and
v. administering a therapeutically effective amount of a pharmaceutical composition;
wherein the composition comprises (a) at least one therapeutic agent, (b) a fatty acid mixture comprising at least one omega-3 fatty acid, and (c) at least one vitamin D compound.
